SEQUENCE LISTING (69 sequences)

SEQ ID NO: 1 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 2 - protein, 7 aa, Homo sapiens
DSTYTQL

SEQ ID NO: 3 - protein, 8 aa, Homo sapiens
PECLSCGS

SEQ ID NO: 4 - protein, 8 aa, Homo sapiens
RICTCRPG

SEQ ID NO: 5 - protein, 5 aa, Homo sapiens
KCSPG

SEQ ID NO: 6 - protein, 17 aa, Homo sapiens
CDSCEDSTYTQLWNWVP

SEQ ID NO: 7 - protein, 18 aa, Homo sapiens
LWNWVPECLSCGSRCSSD

SEQ ID NO: 8 - protein, 20 aa, Homo sapiens
QTAQMCCSKCSPGQHAKVFC

SEQ ID NO: 9 - protein, 5 aa, Homo sapiens
KCRPG

SEQ ID NO: 10 - protein, 120 aa, Homo sapiens
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 11 - protein, 109 aa, Homo sapiens
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 12 - protein, 5 aa, Mus musculus
ADAAP

SEQ ID NO: 13 - protein, 5 aa, Homo sapiens
TVAAP

SEQ ID NO: 14 - protein, 11 aa, Mus musculus
ADAAPTVSIFP

SEQ ID NO: 15 - protein, 12 aa, Homo sapiens
TVAAPSVFIFPP

SEQ ID NO: 16 - protein, 6 aa, Homo sapiens
QPKAAP

SEQ ID NO: 17 - protein, 13 aa, Homo sapiens
QPKAAPSVTLFPP

SEQ ID NO: 18 - protein, 5 aa, synthetic construct
GGSGG

SEQ ID NO: 19 - protein, 9 aa, synthetic construct
GGSGGGGSG

SEQ ID NO: 20 - protein, 13 aa, synthetic construct
GGSGGGGSGGGGS

SEQ ID NO: 21 - protein, 6 aa, Mus musculus
AKTTAP

SEQ ID NO: 22 - protein, 6 aa, Homo sapiens
ASTKGP

SEQ ID NO: 23 - protein, 13 aa, Mus musculus
AKTTAPSVYPLAP

SEQ ID NO: 24 - protein, 13 aa, Homo sapiens
ASTKGPSVFPLAP

SEQ ID NO: 25 - protein, 6 aa, synthetic construct
GGGGSG

SEQ ID NO: 26 - protein, 10 aa, synthetic construct
GGGGSGGGGS

SEQ ID NO: 27 - protein, 14 aa, synthetic construct
GGGGSGGGGSGGGG

SEQ ID NO: 28 - protein, 458 aa, Bison bison
MAPTAFWAALAVGLQFWAAGRAVPAQAVFTPYIPEPGSSCRQQEYYNHKIQMCCSKCPPGYRVQSLCNTTLDTICASCESSTYTQLWNLVTACFSCNSRCSSDQVETQACTTKQNRICTCKPGWYCTLGRQEGCRLCVALRKCGPGFGVAKPGTATTNVICAPCGPGTFSDTTSYTDTCKPHRNCSSVAIPGTASTDAVCTSVLPTRKVARGPATTRSQHMEPTLGPSTAPSTFFLLPKVPSPPSSPVEQPNAGNISLPIELIVGVTALGLLLIVVVNCVIMTQKKKKPFCLQGDAKVPHLPANKAQGAPGPEQQHLLTTAPSSSSSSLESSTSSTDKRAPTRSQLQSPGVEANTSGEAQTGCSSSEASSGGHGTQVNVTCIVNVCSGPDHSSQCPSQAGSTRDTDASTPNSPKEEQVPFSKEERPFQSQPGAPETLLQGLEEKPLPLGVPDAGMKPS

SEQ ID NO: 29 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 30 - protein, 474 aa, Rattus norvegicus
MAPAALWVALVVELQLWATGHTVPAKVVLTPYKPEPGNQCQISQEYYDKKAQMCCAKCPPGQYAKHFCNKTSDTVCADCAAGMFTQVWNHLHTCLSCSSSCSDDQVETHNCTKKQNRVCACNADSYCALKLHSGNCRQCMKLSKCGPGFGVARSRTSNGNVICSACAPGTFSDTTSSTDVCRPHRICSILAIPGNASTDAVCASESPTPSAVPRTIYVSQPEPTRSQPMDQEPGPSQTPHIPVSLGSTPIIEPSITGGISLPIGLIVGLTTLGLLMLGLANCFILVQRKKKPSCLQRETMVPHLPDDKSQDAIGLEQQHLLTTAPSSSSSSLESSASAGDRRAPPGGHPQARVTAEAQGSQEACAGSRSSDSSHGSHGTHVNVTCIVNVCSSSDHSSQCSSQASTTVGDPDANPSGSPKDEQVPFSQEECPSQSQWETTETLQNHDKPFPLGVPDVGMKPNQPGWYDQIAVKVP

SEQ ID NO: 31 - protein, 11 aa, Homo sapiens
SSTDICRPHQI

SEQ ID NO: 32 - protein, 15 aa, Homo sapiens
CALSKQEGCRLCAPL

SEQ ID NO: 33 - protein, 10 aa, Homo sapiens
TSDVVCKPCA

SEQ ID NO: 34 - protein, 459 aa, Bos taurus
MAPTAFWAALAVGLQFWAAGRAVPAQAVFTPYIPEPGSSCRQQEYYNQKIQMCCSKCPPGYRVQSLCNMTLDTICASCESSTYTQLWNLVTACFSCNSRCSSDQVETQACTTKQNRICTCKPGWYCTLGRQEGCRLCVALRKCGPGFGVAKPGTATTNVICAPCGPGTFSDTTSYTDTCKPHRNCSSVAIPGTASTDAVCTSVLPTRKVARGPATTRSQHMEPTLGPSTAPSTFFLLPKVPSPPSSPVEQPNTGNISLPIELIVGVTALGLLLIVVVNCVIMTQKKKKPFCLQGDAKVPHLPANKAQGAPGPEQQHLLTTAPSSSSSSLESSTSSTDKRAPTRSQLQSPGVEKASTSGEAQTGCSSSEASSGGHGTQVNVTCIVNVCSGPDHSSQCPSQAGSTRDTDASTPNSPKEEQVPFSKEERPFQSQPGAPETLLQGLEEKPLPLGVPDAGMKPS

SEQ ID NO: 35 - protein, 14 aa, synthetic construct
  VARIANT 1: note = MISC_FEATURE - Xaa is any amino acid
  VARIANT 3: note = MISC_FEATURE - Xaa is any amino acid
  VARIANT 6: note = MISC_FEATURE - Xaa is any amino acid
  VARIANT 8: note = MISC_FEATURE - Xaa is any amino acid
  VARIANT 10..11: note = MISC_FEATURE - Xaa is any amino acid
  VARIANT 14: note = MISC_FEATURE - Xaa is any amino acid
XRXDGXSXYXXFDX

SEQ ID NO: 36 - protein, 14 aa, synthetic construct
  VARIANT 1: note = MISC_FEATURE - Xaa is any amino acid
  VARIANT 3: note = MISC_FEATURE - Xaa is any amino acid
  VARIANT 8..9: note = MISC_FEATURE - Xaa is any amino acid
  VARIANT 12..14: note = MISC_FEATURE - Xaa is any amino acid
XRXDGSYXXFDXXX

SEQ ID NO: 37 - protein, 14 aa, synthetic construct
  VARIANT 2: note = MISC_FEATURE - Xaa is Arg, Lys, or His
  VARIANT 4: note = MISC_FEATURE - Xaa is Asp or Glu
  VARIANT 5: note = MISC_FEATURE - Xaa is Gly or Ala
  VARIANT 7: note = MISC_FEATURE - Xaa is Asn, Gln, Ser, or Thr
  VARIANT 9: note = MISC_FEATURE - Xaa is Ala, Val, Leu, Ile, Pro, Met, Trp, Phe, or Tyr
  VARIANT 12: note = MISC_FEATURE - Xaa is Ala, Val, Leu, Ile, Pro, Met, Trp, Phe, or Tyr
  VARIANT 13: note = MISC_FEATURE - Xaa is Asp or Glu
  VARIANT 14: note = MISC_FEATURE - Xaa is Ala, Val, Leu, Ile, Pro, Met, Trp, Phe, or Tyr
QXVXXYXSXWYXXX

SEQ ID NO: 38 - protein, 14 aa, synthetic construct
  VARIANT 2: note = MISC_FEATURE - Xaa is Lys, Arg, or His
  VARIANT 4: note = MISC_FEATURE - Xaa is Asp or Glu
  VARIANT 5: note = MISC_FEATURE - Xaa is Gly or Ala
  VARIANT 6: note = MISC_FEATURE - Xaa is Asn, Gln, Ser, or Thr
  VARIANT 7: note = MISC_FEATURE - Xaa is Ala, Val, Leu, Ile, Pro, Met, Trp, Phe, or Tyr
  VARIANT 10: note = MISC_FEATURE - Xaa is Ala, Val, Leu, Ile, Pro, Met, Trp, Phe, or Tyr
  VARIANT 11: note = MISC_FEATURE - Xaa is Asp or Glu
  VARIANT 12: note = MISC_FEATURE - Xaa is Ala, Val, Leu, Ile, Pro, Met, Trp, Phe, or Tyr
AXDXXXXSPXXXWG

SEQ ID NO: 39 - protein, 14 aa, Mus musculus
QRVDGYSSYWYFDV

SEQ ID NO: 40 - protein, 14 aa, Mus musculus
ARDDGSYSPFDYWG

SEQ ID NO: 41 - protein, 14 aa, Mus musculus
ARDDGSYSPFDYFG

SEQ ID NO: 42 - protein, 8 aa, synthetic construct
  VARIANT 2: note = MISC_FEATURE - Xaa is any amino acid
  VARIANT 5..6: note = MISC_FEATURE - Xaa is any amino acid
  VARIANT 8: note = MISC_FEATURE - Xaa is any amino acid
GXTFXXYX

SEQ ID NO: 43 - protein, 8 aa, synthetic construct
  VARIANT 6: note = MISC_FEATURE - Xaa is Gly or Ala
  VARIANT 7: note = MISC_FEATURE - Xaa is Asn, Gln, Ser, or Thr
VDPEYXXT

SEQ ID NO: 44 - protein, 6 aa, synthetic construct
  VARIANT 6: note = MISC_FEATURE - Xaa is Ala, Val, Leu, Ile, Pro, Met, Trp, Phe, or Tyr
QNINKX

SEQ ID NO: 45 - protein, 9 aa, synthetic construct
  VARIANT 4: note = MISC_FEATURE - Xaa is Ala, Val, Leu, Ile, Pro, Met, Trp, Phe, or Tyr
  VARIANT 8: note = MISC_FEATURE - Xaa is Leu or Ile
  VARIANT 9: note = MISC_FEATURE - Xaa is Asn, Gln, Ser, or Thr
CLQXVNLXX

SEQ ID NO: 46 - protein, 8 aa, Mus musculus
GYTFTDYL

SEQ ID NO: 47 - protein, 8 aa, Mus musculus
VDPEYGST

SEQ ID NO: 48 - protein, 6 aa, Mus musculus
QNINKY

SEQ ID NO: 49 - protein, 9 aa, Mus musculus
  VARIANT 8: note = MISC_FEATURE - Xaa is Leu or Ile
CLQYVNLXT

SEQ ID NO: 50 - protein, 7 aa, Mus musculus
  VARIANT 2: note = MISC_FEATURE - Xaa is any amino acid
  VARIANT 5..6: note = MISC_FEATURE - Xaa is any amino acid
GXTFXXY

SEQ ID NO: 51 - protein, 8 aa, Mus musculus
  VARIANT 2: note = MISC_FEATURE - Xaa is any amino acid
  VARIANT 5..6: note = MISC_FEATURE - Xaa is any amino acid
  VARIANT 8: note = MISC_FEATURE - Xaa is any amino acid
GXTFXXYX

SEQ ID NO: 52 - protein, 6 aa, Mus musculus
  VARIANT 4: note = MISC_FEATURE - Xaa is Gly or Ala
  VARIANT 5: note = MISC_FEATURE - Xaa is Asn, Gln, Ser, or Thr
SSGXXY

SEQ ID NO: 53 - protein, 10 aa, Mus musculus
  VARIANT 10: note = MISC_FEATURE - Xaa is Ala, Val, Leu, Ile, Pro, Met, Trp, Phe, or Tyr
SASSSVYYMX

SEQ ID NO: 54 - protein, 7 aa, Mus musculus
  VARIANT 7: note = MISC_FEATURE - Xaa is Asn, Gln, Ser, or Thr
STSNLAX

SEQ ID NO: 55 - protein, 9 aa, Mus musculus
  VARIANT 6: note = MISC_FEATURE - Xaa is Ala, Val, Leu, Ile, Pro, Met, Trp, Phe, or Tyr
  VARIANT 9: note = MISC_FEATURE - Xaa is Asn, Gln, Ser, or Thr
QQRRNXPYX

SEQ ID NO: 56 - protein, 7 aa, Mus musculus
GFTFSSY

SEQ ID NO: 57 - protein, 8 aa, Mus musculus
  VARIANT 8: note = MISC_FEATURE - Xaa is Leu or Ile
GYTFTDYX

SEQ ID NO: 58 - protein, 6 aa, Mus musculus
SSGGSY

SEQ ID NO: 59 - protein, 10 aa, Mus musculus
SASSSVYYMY

SEQ ID NO: 60 - protein, 7 aa, Mus musculus
STSNLAS

SEQ ID NO: 61 - protein, 9 aa, Mus musculus
QQRRNYPYT

SEQ ID NO: 62 - protein, 8 aa, Mus musculus
GYTFTDYI

SEQ ID NO: 63 - protein, 8 aa, Mus musculus
GYTFTDVI

SEQ ID NO: 64 - protein, 8 aa, Mus musculus
GYTFTDYS

SEQ ID NO: 65 - protein, 9 aa, Mus musculus
CLQYVNLLT

SEQ ID NO: 66 - protein, 9 aa, Mus musculus
CLQYVNLIT

SEQ ID NO: 67 - protein, 4 aa, Mus musculus
LLIR

SEQ ID NO: 68 - protein, 123 aa, Mus musculus
EVQLQESGGGLVKPGGSLKLSCAASGFTFSSYVMSWVRQTPEKRLEWVATISSGGSYTYYPDSVKGRFTISRDNAKNTLYLQMSSLRSEDTAMYYCARQRVDGYSSYWYFDVWGAGTAVTVSS

SEQ ID NO: 69 - protein, 108 aa, Mus musculus
DIVLTQSPAIMSASPGEKVTITCSASSSVYYMYWFQQKPGTSPKLWIYSTSNLASGVPVRFSGSGSGTSYSLTISRMEAEDAATYYCQQRRNYPYTFGGGTKLEIKRA

(19) United States
(12) Patent Application Publication
(10) Pub. No.: US 2023/0383003 A1
(43) Pub. Date: Nov. 30, 2023

(54) ANTAGONISTIC ANTI-TUMOR NECROSIS FACTOR RECEPTOR SUPERFAMILY POLYPEPTIDES

(71) Applicant: The General Hospital Corporation, Boston, MA (US)

(72) Inventor: Denise L. FAUSTMAN, Boston, MA (US)

(21) Appl. No.: 18/334,574

(22) Filed: Jun. 14, 2023

Related U.S. Application Data
(62) Division of application No. 16/762,038, filed on May 6, 2020.
(60) Provisional application No. 62/647,254, filed on Mar. 23, 2018.
(60) Provisional application No. 62/583,897, filed on Nov. 9, 2017.

(51) Int. Cl. C07K 16/28; A61K 47/68; A61P 31/18; A61P 31/06; A61P 35/00; A61K 39/395; C12N 5/071; C12N 7/00; C12N 15/86; G01N 33/543; G01N 33/58; G01N 33/68

(52) U.S. Cl. CPC C07K 16/2878; A61K 47/6801; A61P 31/18; A61P 31/06; A61P 35/00; A61K 39/3955; C12N 5/0682; C12N 7/00; C12N 15/86; G01N 33/543; G01N 33/58; G01N 33/6818; G01N 33/6854; A61K 2039/505

(57) ABSTRACT

Described are antagonistic TNFR2 polypeptides, such as antibodies and antigen-binding fragments thereof, and the use of these polypeptides to inhibit the proliferation of regulatory T cells (T-regs) and/or myeloid-derived suppressor cells (MDSCs), to expand T effector cell populations or function, and to reduce the proliferation of, or directly kill, tumor cells, such as tumor cells that express TNFR2 antigen. The polypeptides, such as antibodies and antigen-binding fragments thereof, are TNFR2 antagonists, such as dominant TNFR2 antagonists. The polypeptides can be used to suppress the T-reg- or MDSC-mediated deactivation of tumor reactive T lymphocytes, expand populations of tumor-reactive cytotoxic T cells, and/or to directly kill TNFR2+ tumor cells. The antagonistic TNFR2 polypeptides described herein can be used to treat a wide variety of cancers and infectious diseases.

### SEQUENCE LISTING

The instant application contains a Sequence Listing which has been submitted electronically in XML format and is hereby incorporated by reference in its entirety. Said XML copy, created on Jun. 13, 2023, is named 00786-580004_SL.xml and is 82,250 bytes in size.

### FIELD OF THE INVENTION

The invention relates to polypeptides, such as antibodies and antigen-binding fragments thereof, capable of antagonizing tumor necrosis factor receptor superfamily members, such as tumor necrosis factor receptor 2. The polypeptides described herein can be used to modulate the activity of regulatory T cells, as well as to upregulate the activity of T effector cells, regulate surface oncogene expression, and directly kill cancer cells. The polypeptides described herein are thus useful, for instance, in the field of immunotherapy for the treatment of cell proliferation disorders and infectious diseases.

### BACKGROUND OF THE INVENTION

The use of naturally-occurring and genetically engineered T lymphocytes is a prominent paradigm for ameliorating various human pathologies. For instance, while traditional therapeutic platforms for the treatment of cancer include surgical removal of tumor mass, radiation therapy, and administration of chemotherapeutics (Shewach, Chem. Rev., 109:2859-2861, 2009), the last decade has witnessed a resurgence in the application of adoptive immunotherapy to cancer treatment regimens. With the advent of chimeric antigen receptor (CAR-T) therapy, new methods have emerged for the infusion of autologous and allogeneic tumor-reactive T cells to patients (June, J. Clin. Invest., 117:1466-1476, 2007). CAR-T therapies harness the resources of the adaptive immune response in order to promote cancer cell cytotoxicity and eradicate tumor material. A common motif in adoptive immunotherapy is the use of T cells that exhibit the ability to selectively potentiate cytotoxicity in cells that display distinct tumor antigens. Examples of this technique include the administration of tumor-infiltrating lymphocytes (Dudley et al., J. Immunother., 26:332-342, 2003), as well as autologous or allogeneic T cells that have been genetically re-engineered so as to exhibit reactivity with a tumor-specific antigen (Yee et al., PNAS., 99:16168-16173, 2002).

Despite the promise of T lymphocyte-based cancer immunotherapy, the development of this therapeutic platform has been hindered by the natural propensity of the immune system to suppress immune attacks mounted on self cells. Cancer cells, like all nucleated human cells, express class I major histocompatability complex (MHC) proteins that distinguish these cells from foreign cells. In order to prevent cell fratricide, regulatory T cells (T-reg cells) have evolved that suppress the activity of T cells that exhibit reactivity against “self” MHC antigens. T-reg cells represent a heterogeneous class of T cells that can be distinguished based on their unique surface protein presentation. The most well-understood populations of T-reg cells include CD4+, CD25+, FoxP3+ T-reg cells and CD17+ T-reg cells. The precise mechanisms by which these cells mediate suppression of autoreactive T cells is the subject of ongoing investigations, though it has been shown that certain classes of T-reg cells inhibit production of the proliferation-inducing cytokine IL-2 in target T cells and may additionally sequester IL-2 from autoreactive cells by virtue of the affinity of CD25 (a subdomain of the IL-2 receptor) for IL-2 (Josefowicz et al., Ann. Rev. Immun., 30:531-564, 2012).

Although T-reg cells play an important role in maintaining peripheral tolerance, the same biochemical features that underlie the ability of these cells to modulate autoreactive T cell activity also serve to undermine adoptive immunotherapy and the natural immune response by suppressing the activity of tumor-reactive T lymphocytes. The development of chemical modulators of T-reg cell activity has been the subject of many pharmacological investigations, as access to an agent capable of inhibiting T-reg-mediated T cell suppression could vastly improve the scope and efficacy of adoptive cancer immunotherapy, as well as improve the ability of the immune system to eradicate pathogenic organisms that give rise to infectious diseases.

There is currently a need for therapies that can prevent T-reg cell survival and proliferation for use in treatments targeting cell proliferation disorders, such as cancer, and a wide array of infectious diseases.

### SUMMARY OF THE INVENTION

Described herein are antagonistic tumor necrosis factor receptor superfamily polypeptides, such as single-chain polypeptides, antibodies, antigen-binding fragments thereof, and constructs. For instance, featured are antagonistic tumor necrosis factor receptor 2 (TNFR2) polypeptides, such as single-chain polypeptides, antibodies, antigen-binding fragments thereof, and constructs. Antagonistic TNFR2 polypeptides described herein specifically bind epitopes within human TNFR2, for instance, that contain one or more residues of the SSTDICRPHQI sequence (SEQ ID NO: 31), the CALSKQEGCRLCAPL sequence (SEQ ID NO: 32), and/or the TSDVVCKPCA sequence (SEQ ID NO: 33) of human TNFR2 (the full amino acid sequence of which is shown in SEQ ID NO: 1) or equivalent epitopes in TNFR2 of non-human primates (e.g., bison or cattle, as described herein). Particular antagonistic TNFR2 polypeptides (e.g., antibodies, antigen-binding fragments thereof, and constructs) are those that do not specifically bind residues of the KCSPG motif (SEQ ID NO: 5) within human TNFR2 or equivalent epitopes in TNFR2 of non-human primates. The polypeptides (e.g., single-chain polypeptides, antibodies, antigen-binding fragments thereof, and constructs) described herein can be used for the treatment of a variety of pathologies, including cancers and infectious diseases.

Antagonistic TNFR2 polypeptides are those that exhibit the ability to inhibit the proliferation of, and/or promote the death of, T-reg cells and/or myeloid-derived suppressor cells (MDSCs). Antagonistic TNFR2 polypeptides may inhibit the proliferation of, and/or promote the death of, TNFR2- and oncogene-expressing cancer cells. Additionally, or alternatively, antagonistic TNFR2 polypeptides can permit the reciprocal expansion of T effector cells, such as cytotoxic CD8+ T cells. This may occur, for instance, by the attenuation of T-reg cell proliferation and activity or by the direct expansion of T effector cells, such as cytotoxic CD8+ T cells. Therefore, the designation of TNFR2 polypeptides as antagonists refers to their capacity to attenuate the proliferation and activity of T-reg cells, MDSCs, and/or TNFR2-expressing cancer cells and, for clarity, does not indicate antagonism of the T effector cell response.

Human TNFR2 contains four cysteine-rich domains (CRDs): CRD1 (amino acid residues 48-76 of SEQ ID NO: 1), CRD2 (amino acid residues 78-120 of SEQ ID NO: 1), CRD3 (amino acid residues 121-162 of SEQ ID NO: 1), and CRD4 (amino acid residues 162-202 of SEQ ID NO: 1). Antagonistic TNFR2 polypeptides described herein include those that bind one or more epitopes within CRD3 of TNFR2 and/or one or more epitopes within CRD4 of TNFR2, such as those that bind TNFR2 exclusively within one or more epitopes of CRD3 and/one or more epitopes of CRD4. Antagonistic TNFR2 polypeptides that bind TNFR2 within CRD3 and CRD4 may exhibit one or more beneficial biological properties, such as the ability to kill and/or inhibit the proliferation of T-reg cells, kill and/or inhibit the proliferation of TNFR2+ cancer cells, kill and/or inhibit the proliferation of myeloid-derive suppressor cells (MDSCs), and/or induce the proliferation of effector T cells.

Antagonistic TNFR2 polypeptides described herein can exhibit one or more, or all, of the foregoing properties without the need to bind an epitope within the KCRPG sequence (SEQ ID NO: 9) of human TNFR2 or an equivalent epitope in TNFR2 of a non-human primate (e.g., bison or cattle, as described herein). Antagonistic TNFR2 polypeptides described herein, thus, include those that bind one or more epitopes within CRD3 of TNFR2 and/or one or more epitopes within CRD4 of TNFR2 and that do not bind to an epitope within amino acids 142-146 (KCRPG) of SEQ ID NO: 1, such as those that bind TNFR2 exclusively within one or more epitopes of CRD3 and/one or more epitopes of CRD4 and that do not bind to an epitope within amino acids 142-146 (KCRPG) of SEQ ID NO: 1 or an equivalent epitope in TNFR2 of a non-human primate (e.g., bison or cattle, as described herein).

In a first aspect, disclosed herein are polypeptides, such as single-chain polypeptides, antibodies, antigen-binding fragments thereof, and constructs, that specifically binds human tumor TNFR2 at an epitope defined by one or more amino acids within CRD3 and/or an epitope defined by one or more amino acids within CRD4. In some embodiments, the polypeptides, such as single-chain polypeptides, antibodies, antigen-binding fragments thereof, and constructs containing the polypeptides, do not bind an epitope of TNFR2 defined by one or more of amino acids 142-146 (KCRPG) of SEQ ID NO: 1 In some embodiments, the polypeptides, such as single-chain polypeptides, antibodies, antigen-binding fragments thereof, and constructs containing the polypeptides, specifically bind human TNFR2 at:
(a) a first epitope of TNFR2 within amino acids 174-184 (SSTDICRPHQI), inclusive of the endpoints, of SEQ ID NO:1;
(b) a second epitope of TNFR2 within amino acids 126-140 (CALSKQEGCRLCAPL), inclusive of the endpoints, of SEQ ID NO: 1; and/or
(c) a third epitope of TNFR2 within amino acids 156-165 (TSDVVCKPCA), inclusive of the endpoints, of SEQ ID NO: 1.

In some embodiments, the polypeptides, such as single-chain polypeptides, antibodies, antigen-binding fragments thereof, and constructs containing the polypeptides, specifically bind human TNFR2 at an epitope of TNFR2 within amino acids 174-184 (SSTDICRPHQI) of SEQ ID NO: 1. The polypeptide, such as a single-chain polypeptide, antibody, antigen-binding fragment thereof, or construct, may specifically bind human TNFR2 at an additional epitope, such as an epitope of TNFR2 within amino acids 126-140 (CALSKQEGCRLCAPL) of SEQ ID NO: 1. Additionally, or alternatively, the polypeptide, such as a single-chain polypeptide, antibody, antigen-binding fragment thereof, or construct, may specifically bind human TNFR2 at an additional epitope of TNFR2 within amino acids 156-165 (TSDVVCKPCA) of SEQ ID NO: 1.

In some embodiments, the polypeptides, such as single-chain polypeptides, antibodies, antigen-binding fragments thereof, and constructs, specifically bind human TNFR2 at an epitope of TNFR2 within amino acids 126-140 (CALSKQEGCRLCAPL) of SEQ ID NO: 1. The polypeptide, such as a single-chain polypeptide, antibody, antigen-binding fragment thereof, or construct, may specifically bind human TNFR2 at an additional epitope, such as an epitope of TNFR2 within amino acids 174-184 (SSTDICRPHQI) of SEQ ID NO: 1. Additionally, or alternatively, the polypeptide, such as a single-chain polypeptide, antibody, antigen-binding fragment thereof, or construct, may specifically bind human TNFR2 at an additional epitope of TNFR2 within amino acids 156-165 (TSDVVCKPCA) of SEQ ID NO: 1.

In some embodiments, the polypeptides, such as single-chain polypeptides, antibodies, antigen-binding fragments thereof, and constructs, specifically bind human TNFR2 at an epitope of TNFR2 within amino acids 156-165 (TSDVVCKPCA) of SEQ ID NO: 1. The polypeptide, such as a single-chain polypeptide, antibody, antigen-binding fragment thereof, or construct, may specifically bind human TNFR2 at an additional epitope, such as an epitope of TNFR2 within amino acids 174-184 (SSTDICRPHQI) of SEQ ID NO: 1. Additionally, or alternatively, the polypeptide, such as a single-chain polypeptide, antibody, antigen-binding fragment thereof, or construct, may specifically bind human TNFR2 at an additional epitope of TNFR2 within amino acids 126-140 (CALSKQEGCRLCAPL) of SEQ ID NO: 1.

In some embodiments, the polypeptide, such as the single-chain polypeptide, antibody, antigen-binding fragment thereof, or construct, specifically binds one or more, or all, of the above epitopes with a K_{D} of less than 100 nM (e.g., with a K_{D} of less than 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, 1 nM, 900 pM, 800 pM, 700 pM, 600 pM, 500 pM, 400 pM, 300 pM, 200 pM, 100 pM, 95 pM, 90 pM, 85 pM, 80 pM, 75 pM, 70 pM, 65 pM, 60 pM, 55 pM, 50 pM, 45 pM, 40 pM, 35 pM, 30 pM, 25 pM, 20 pM, 15 pM, 10 pM, 5 pM, 4 pM, 3 pM, 2 pM, 1 pM, or less). For instance, the polypeptide, such as the single-chain polypeptide, antibody, antigen-binding fragment thereof, or construct, may specifically bind one or more, or all, of the above epitopes with a K_{D} of less than 10 nM (e.g., e.g., with a K_{D} of less than 5 nM, 4 nM, 3 nM, 2 nM, 1 nM, 900 pM, 800 pM, 700 pM, 600 pM, 500 pM, 400 pM, 300 pM, 200 pM, 100 pM, 95 pM, 90 pM, 85 pM, 80 pM, 75 pM, 70 pM, 65 pM, 60 pM, 55 pM, 50 pM, 45 pM, 40 pM, 35 pM, 30 pM, 25 pM, 20 pM, 15 pM, 10 pM, 5 pM, 4 pM, 3 pM, 2 pM, 1 pM, or less).

In some embodiments, the polypeptide (such as the single-chain polypeptide, antibody, antigen-binding fragment thereof, or construct) does not bind an epitope of TNFR2 within amino acids 142-146 (KCRPG) of SEQ ID NO: 1. Additionally, or alternatively, the polypeptide (such as the single-chain polypeptide, antibody, antigen-binding fragment thereof, or construct) may:
(a) not bind an epitope of TNFR2 within amino acids 80-86 (DSTYTQL) of SEQ ID NO: 1;
(b) not bind an epitope of TNFR2 within amino acids 75-91 (CDSCEDSTYTQLWNWVP) of SEQ ID NO: 1;
(c) not bind an epitope of TNFR2 within amino acids 91-98 (PECLSCGS) of SEQ ID NO: 1;
(d) not bind an epitope of TNFR2 within amino acids 86-103 (LWNWVPECLSCGSRCSSD) of SEQ ID NO: 1;
(e) not bind an epitope of TNFR2 within amino acids 116-123 (RICTCRPG) of SEQ ID NO: 1; and/or
(f) not bind an epitope of TNFR2 within amino acids 56-60 (KCSPG) of SEQ ID NO: 1.

In some embodiments, the polypeptide (such as the single-chain polypeptide, antibody, antigen-binding fragment thereof, or construct) contains a non-native constant region, such as a human constant region, lacks all or a portion of an Fc domain, lacks all or a portion of a native Fc domain, or lacks an Fc domain altogether.

The antagonistic TNFR2 polypeptides, such as dominant antagonistic TNFR2 polypeptides, of the disclosure (e.g., a single-chain polypeptide, antibody, antigen-binding fragment thereof, or construct thereof described herein) may contain a complementarity-determining region (CDR) heavy chain 1 (CDR1) having the amino acid sequence GJTF(J)₂Y (SEQ ID NO: 50) or GJTF(J)₂YJ (SEQ ID NO: 51), in which each J is independently a naturally occurring amino acid. In some embodiments, the polypeptide (e.g., a single-chain polypeptide, antibody, antigen-binding fragment thereof, or construct thereof) further contains:
(a) a CDR-H2 having the amino acid sequence (J)₃GSJ or (J)₅GSJ;
(b) a CDR-H3 having the amino acid sequence JRJDGJSJY(J)₂FDJ (SEQ ID NO: 35) or JRJDGSY(J)₂FD(J)₃ (SEQ ID NO: 36);
(c) a CDR-L1 having the amino acid sequence (J)₉Y or (J)₅Y;
(d) a CDR-L2 having the amino acid sequence (J)₆S or (J)₂S; and/or
(e) a CDR-L3 having the amino acid sequence (J)₅Y(J)₂T or (J)₃Y(J)₄T, in which each J is independently a naturally occurring amino acid.

The polypeptide (e.g., a single-chain polypeptide, antibody, antigen-binding fragment thereof, or construct thereof) may contain a CDR-H1 having the amino acid sequence Z⁴FZ³Z⁵SSZ⁵ or Z⁴YZ³Z⁵TDZ⁵X;

In which each Z³ is independently an amino acid including a polar, uncharged side-chain at physiological pH;

each Z⁴ is independently a glycine or alanine;

each Z⁵ is independently an amino acid including a hydrophobic side-chain; and

each X is independently leucine or isoleucine.

In some embodiments, the polypeptide (e.g., a single-chain polypeptide, antibody, antigen-binding fragment thereof, or construct thereof) further contains:
(a) a CDR-H2 having the amino acid sequence SSGZ⁴Z³Y (SEQ ID NO: 52) or VDPEYZ⁴Z³T (SEQ ID NO: 43);
(b) a CDR-H3 having the amino acid sequence QZ¹VZ²Z⁴YZ³SZ⁵WYZ⁵Z²Z⁵ (SEQ ID NO: 37) or AZ¹DZ²Z⁴Z³Z⁵SPZ⁵Z²Z⁵WG (SEQ ID NO: 38);
(c) a CDR-L1 having the amino acid sequence SASSSVYYMZ⁵ (SEQ ID NO: 53) or QNINKZ⁵ (SEQ ID NO: 44);
(d) a CDR-L2 having the amino acid sequence STSNLAZ³ (SEQ ID NO: 54), TYZ³, or YTZ³; and/or
(e) a CDR-L3 having the amino acid sequence QQRRNZ⁵PYZ³ (SEQ ID NO: 55) or CLQZ⁵VNLXZ³ (SEQ ID NO: 45);
in which each Z¹ is independently an amino acid including a cationic side-chain at physiological pH;
each Z² is independently an amino acid including an anionic side-chain at physiological pH;
each Z³ is independently an amino acid including a polar, uncharged side-chain at physiological pH;
each Z⁴ is independently a glycine or alanine;
each Z⁵ is independently an amino acid including a hydrophobic side-chain; and
each X is independently leucine or isoleucine.

The antagonistic TNFR2 polypeptides (e.g., a dominant antagonistic TNFR2 polyleptide) of the disclosure (e.g., a single-chain polypeptide, antibody, antigen-binding fragment thereof, or construct thereof) may contain a CDR-H1 having the amino acid sequence GFTFSSY (SEQ ID NO: 56), GYTFTDYX (SEQ ID NO: 57), or an amino acid sequence having up to two amino acid substitutions (e.g., conservative amino acid substitutions) relative to these sequences, in which each X is independently leucine or isoleucine, optionally in which the amino acid substitutions are conservative amino acid substitutions. In some embodiments, the polypeptide (e.g., a single-chain polypeptide, antibody, antigen-binding fragment thereof, or construct thereof) further contains:
(a) a CDR-H2 having the amino acid sequence SSGGSY (SEQ ID NO: 58), VDPEYGST (SEQ ID NO: 47), or an amino acid sequence having up to two amino acid substitutions (e.g., conservative amino acid substitutions) relative to these sequences;
(b) a CDR-H3 having the amino acid sequence QRVDGYSSYWYFDV (SEQ ID NO: 39), ARDDGSYSPFDYWG (SEQ ID NO: 40), or an amino acid sequence having up to two amino acid substitutions (e.g., conservative amino acid substitutions) relative to these sequences;
(c) a CDR-L1 having the amino acid sequence SASSSVYYMY (SEQ ID NO: 59), QNINKY (SEQ ID NO: 48), or an amino acid sequence having up to two amino acid substitutions (e.g., conservative amino acid substitutions) relative to these sequences;
(d) a CDR-L2 having the amino acid sequence STSNLAS (SEQ ID NO: 60), TYS, YTS, or an amino acid sequence having up to two amino acid substitutions (e.g., conservative amino acid substitutions) relative to SEQ ID NO: 60; and/or
(e) a CDR-L3 having the amino acid sequence QQRRNYPYT (SEQ ID NO: 61), CLQYVNLXT (SEQ ID NO: 49), or an amino acid sequence having up to two amino acid substitutions (e.g., conservative amino acid substitutions) relative to these sequences.

In some embodiments, the polypeptide (e.g., a single-chain polypeptide, antibody, antigen-binding fragment thereof, or construct thereof) contains a heavy chain including one or more of the following CDRs:
(a) a CDR-H1 having the amino acid sequence GFTFSSY (SEQ ID NO: 56);
(b) a CDR-H2 having the amino acid sequence SSGGSY (SEQ ID NO: 58); and
(c) a CDR-H3 having the amino acid sequence QRVDGYSSYWYFDV (SEQ ID NO: 39).

The polypeptide (e.g., a single-chain polypeptide, antibody, antigen-binding fragment thereof, or construct thereof) may contain, for example, a heavy chain having one or more of the following CDRs:
(a) a CDR-H1 having the amino acid sequence GYTFTDYX (SEQ ID NO: 57);
(b) a CDR-H2 having the amino acid sequence VDPEYGST (SEQ ID NO: 47); and
(c) a CDR-H3 having the amino acid sequence ARDDGSYSPFDYWG (SEQ ID NO: 40);
in which each X is independently leucine or isoleucine.

In some embodiments, the CDR-H1 has the amino acid sequence GYTFTDYL (SEQ ID NO: 46). In some embodiments, the CDR-H1 has the amino acid sequence GYTFTDYI (SEQ ID NO: 62). In some embodiments, the CDR-H1 has the amino acid sequence GYTFTDVI (SEQ ID NO: 63). In some embodiments, the CDR-H1 has the amino acid sequence GYTFTDYS (SEQ ID NO: 64).

Additionally or alternatively, the polypeptide (e.g., a single-chain polypeptide, antibody, antigen-binding fragment thereof, or construct thereof) may contain, for example, a light chain having one or more of the following CDRs:
(a) a CDR-L1 having the amino acid sequence SASSSVYYMY (SEQ ID NO: 59);
(b) a CDR-L2 having the amino acid sequence STSNLAS (SEQ ID NO: 60); and
(c) a CDR-L3 having the amino acid sequence QQRRNYPYT (SEQ ID NO: 61).

In some embodiments, the antibody or antigen-binding fragment thereof contains a light chain having one or more of the following CDRs:
(a) a CDR-L1 having the amino acid sequence QNINKY (SEQ ID NO: 48);
(b) a CDR-L2 having the amino acid sequence TYS or YTS; and
(c) a CDR-L3 having the amino acid sequence CLQYVNLXT (SEQ ID NO: 49);
in which each X is independently leucine or isoleucine.

In some embodiments, the CDR-L2 has the amino acid sequence TYS. In some embodiments, the CDR-L2 has the amino acid sequence YTS. The CDR-L3 may have the amino acid sequence CLQYVNLLT (SEQ ID NO: 65). In some embodiments, the CDR-L3 has the amino acid sequence CLQYVNLIT (SEQ ID NO: 66).

The polypeptide (e.g., a single-chain polypeptide, antibody, antigen-binding fragment thereof, or construct thereof) may contain three heavy chain CDRs, including:
(a) a CDR-H1 having the amino acid sequence GFTFSSY (SEQ ID NO: 56);
(b) a CDR-H2 having the amino acid sequence SSGGSY (SEQ ID NO: 58); and
(c) a CDR-H3 having the amino acid sequence QRVDGYSSYWYFDV (SEQ ID NO: 39);
and may further contain three light chain CDRs, including:
(d) a CDR-L1 having the amino acid sequence SASSSVYYMY (SEQ ID NO: 59);
(e) a CDR-L2 having the amino acid sequence STSNLAS (SEQ ID NO: 60); and
(f) a CDR-L3 having the amino acid sequence QQRRNYPYT (SEQ ID NO: 61).

In some embodiments, polypeptide (e.g., single-chain polypeptides, antibody, antigen-binding fragment thereof, or construct thereof) contains three heavy chain CDRs, including:
(a) a CDR-H1 having the amino acid sequence GYTFTDYX (SEQ ID NO: 57), such as GYTFTDYL (SEQ ID NO: 46) or GYTFTDYI (SEQ ID NO: 62);
(b) a CDR-H2 having the amino acid sequence VDPEYGST (SEQ ID NO: 47); and
(c) a CDR-H3 having the amino acid sequence ARDDGSYSPFDYWG (SEQ ID NO: 40);
and further contains three light chain CDRs, including:
(d) a CDR-L1 having the amino acid sequence QNINKY (SEQ ID NO: 48);
(e) a CDR-L2 having the amino acid sequence TYS or YTS; and
(f) a CDR-L3 having the amino acid sequence CLQYVNLXT (SEQ ID NO: 49), such as CLQYVNLLT (SEQ ID NO: 65) or CLQYVNLIT (SEQ ID NO: 66);
in which each X is independently leucine or isoleucine.

In some embodiments, the polypeptide (e.g., a single-chain polypeptide, antibody, antigen-binding fragment thereof, or construct thereof) includes a framework region having the amino acid sequence LLIR (SEQ ID NO: 67) bound to the N-terminus of the CDR-L2 and/or a framework region having the amino acid sequence TLE bound to the C-terminus of the CDR-L2.

The antagonistic TNFR2 polypeptides (e.g., a dominant antagonistic TNFR2 polyleptide) of the disclosure (e.g., a single-chain polypeptide, antibody, antigen-binding fragment thereof, or construct thereof) may have a heavy chain variable domain having an amino acid sequence that is at least 85% identical (e.g., at least 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 68. In some embodiments, the heavy chain variable domain has an amino acid sequence that is at least 90% identical (e.g., at least 90%, 91%, 92%, 93%, 94%, 95%, 96%, 97%, 98%, 99%, 99.9%, or 100% identical) to the amino acid sequence of SEQ ID NO: 68. In some embodiments, the heavy chain variable domain has an amino acid sequence that is at least 95% identical (e.g., at least 95%, 96%, 97%, 98%, 99%, 99.9%, or 100% identical) to the amino acid sequence of SEQ ID NO: 68.

Additionally or alternatively, the polypeptide (e.g., a single-chain polypeptide, antibody, antigen-binding fragment thereof, or construct thereof) may have a light chain variable domain having an amino acid sequence that is at least 85% identical (e.g., at least 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 69. In some embodiments, the light chain variable domain has an amino acid sequence that is at least 90% identical (e.g., at least 90%, 91%, 92%, 93%, 94%, 95%, 96%, 97%, 98%, 99%, 99.9%, or 100% identical) to the amino acid sequence of SEQ ID NO: 69. In some embodiments, the light chain variable domain has an amino acid sequence that is at least 95% identical (e.g., at least 95%, 96%, 97%, 98%, 99%, 99.9%, or 100% identical) to the amino acid sequence of SEQ ID NO: 69.

The polypeptides (e.g., single-chain polypeptides, antibodies, or antigen-binding fragments thereof), described herein may contain a CDR-H3 represented by the formula JZ¹JZ²Z⁴JZ³JZ⁵(J)₂Z⁵Z²Z⁵ or JZ¹JZ²Z⁴Z³Z⁵(J)₂Z⁵Z²Z⁵(J)₂, wherein each J is independently a naturally occurring amino acid, each Z¹ is independently a naturally occurring amino acid containing a cationic side-chain at physiological pH, each Z² is independently a naturally occurring amino acid containing an anionic side-chain at physiological pH, each Z³ is independently a naturally occurring amino acid containing a polar, uncharged side-chain at physiological pH, each Z⁴ is independently a glycine or alanine, and each Z⁵ is independently a naturally occurring amino acid containing a hydrophobic side-chain.

Similarly, the antagonistic TNFR2 polypeptides described herein, such as dominant antagonistic TNFR2 polypeptides (e.g., single-chain polypeptides, antibodies, or antigen-binding fragments thereof), may contain a CDR-H3 represented by the formula JRJDGJSJY(J)₂FDJ (SEQ ID NO: 35), JRJDGSY(J)₂FD(J)₃ (SEQ ID NO: 36), QZ¹VZ²Z⁴YZ³SZ⁵WYZ⁵Z²Z⁵ (SEQ ID NO: 37), or AZ¹DZ²Z⁴Z³Z⁵SPZ⁵Z²Z⁵WG (SEQ ID NO: 38). For instance, the CDR-H3 may have the amino acid sequence QRVDGYSSYWYFDV (SEQ ID NO: 39).

The CDR-H3 may have the amino acid sequence ARDDGSYSPFDYWG (SEQ ID NO: 40). In some embodiments, the CDR-H3 has the amino acid sequence ARDDGSYSPFDYFG (SEQ ID NO: 41).

Additionally, or alternatively, the antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence Z⁴JZ³Z⁵(J)₂Z⁵J; a CDR-H2 having the amino acid sequence (J)₅Z⁴Z³J; a CDR-L1 having the amino acid sequence (J)₅Z⁵; a CDR-L2 having the amino acid sequence (J)₂Z³; and/or a CDR-L3 having the amino acid sequence (J)₃Z⁵(J)₄Z³; wherein each J is independently a naturally occurring amino acid; each Z¹ is independently a naturally occurring amino acid containing a cationic side-chain at physiological pH; each Z² is independently a naturally occurring amino acid containing an anionic side-chain at physiological pH; each Z³ is independently a naturally occurring amino acid containing a polar, uncharged side-chain at physiological pH; each Z⁴ is independently a glycine or alanine; and each Z⁵ is independently a naturally occurring amino acid containing a hydrophobic side-chain.

In some embodiments, the antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence GJTF(J)₂YL (SEQ ID NO: 42); a CDR-H2 having the amino acid sequence (J)₅GSJ; a CDR-L1 having the amino acid sequence (J)₅Y; a CDR-L2 having the amino acid sequence (J)₂S; and/or a CDR-L3 having the amino acid sequence (J)₃Y(J)₄T; wherein each J is independently a naturally occurring amino acid.

The antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence Z⁴YZ³Z⁵TDZ⁵L; a CDR-H2 having the amino acid sequence VDPEYZ⁴Z³T (SEQ ID NO: 43); a CDR-L1 having the amino acid sequence QNINKZ⁵ (SEQ ID NO: 44); a CDR-L2 having the amino acid sequence TYZ³ or YTZ³; and/or a CDR-L3 having the amino acid sequence CLQZ⁵VNLXZ³ (SEQ ID NO: 45); wherein each Z¹ is independently an amino acid containing a cationic side-chain at physiological pH; each Z² is independently an amino acid containing an anionic side-chain at physiological pH; each Z³ is independently an amino acid containing a polar, uncharged side-chain at physiological pH; each Z⁴ is independently a glycine or alanine; each Z⁵ is independently an amino acid containing a hydrophobic side-chain; and each X is independently leucine or isoleucine.

In some embodiments, the antagonistic TNFR2 polypeptides described herein, such as dominant antagonistic TNFR2 polypeptides (e.g., single-chain polypeptides, antibodies, or antigen-binding fragments thereof), may have a CDR-H1 having the amino acid sequence GYTFTDYL (SEQ ID NO: 46), or an amino acid sequence having up to two amino acid substitutions relative to this sequence, provided that the CDR-H1 preserves the C-terminal leucine residue of SEQ ID NO: 46; a CDR-H2 having the amino acid sequence VDPEYGST (SEQ ID NO: 47), or an amino acid sequence having up to two amino acid substitutions relative to this sequence; a CDR-L1 having the amino acid sequence QNINKY (SEQ ID NO: 48), or an amino acid sequence having up to two amino acid substitutions relative to this sequence; a CDR-L2 having the amino acid sequence TYS or YTS; and/or a CDR-L3 having the amino acid sequence CLQYVNLXT (SEQ ID NO: 49), or an amino acid sequence having up to two amino acid substitutions relative to this sequence.

A second aspect features constructs containing a first polypeptide domain and a second polypeptide domain, each of which contains a single-chain polypeptide, such as those defined in the first aspect. The first and second polypeptide domains may be the same or different. The first and second polypeptide domains may be bound, for instance, by a linker, such as a linker containing an amide bond or a disulfide bridge. The constructs may lack a murine Fc domain.

In some embodiments, the polypeptides (e.g., single-chain polypeptides, antibodies, antigen-binding fragments thereof, or constructs) described herein bind TNFR2 with a K_{D} of no greater than 100 nM (e.g., with a K_{D} of 100 nM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 1 nM, 900 pM, 800 pM, 700 pM, 600 pM, 500 pM, 400 pM, 300 pM, 200 pM, 100 pM, or less). The polypeptides may bind TNFR2, for example, with a K_{D} of no greater than 10 nM (e.g., with a K_{D} of 10 nM, 5 nM, 1 nM, 900 pM, 800 pM, 700 pM, 600 pM, 500 pM, 400 pM, 300 pM, 200 pM, 100 pM, or less). Additionally, or alternatively, the polypeptides described herein may bind TNFR2 with a kₒₙ of, for example, at least about 10⁴ M⁻¹s⁻¹ (e.g., with a kₒₙ of about 1×10⁴ M⁻¹s⁻¹, 2×10⁴ M⁻¹s⁻¹, 3×10⁴ M⁻¹s⁻¹, 4×10⁴ M⁻¹s⁻¹, 5×10⁴ M⁻¹s⁻¹, 6×10⁴ M⁻¹s⁻¹, 7×10⁴ M⁻¹s⁻¹, 8×10⁴ M⁻¹s⁻¹, 9×10⁴ M⁻¹s⁻¹, 1×10⁵ M⁻¹s⁻¹, 2×10⁵ M⁻¹s⁻¹, 3×10⁵ M⁻¹s⁻¹, 4×10⁵ M⁻¹s⁻¹, 5×10⁵ M⁻¹s⁻¹, 6×10⁵ M⁻¹s⁻¹, 7×10⁵ M⁻¹s⁻¹, 8×10⁵ M⁻¹s⁻¹, 9×10⁵ M⁻¹s⁻¹, 1×10⁶ M⁻¹s⁻¹, or greater). The polypeptides described herein may bind TNFR2 and dissociate with a kₒₙ of, for instance, no greater than about 10-3 s⁻¹ (e.g., with a kₒₙ of about 1×10⁻³ s⁻¹, 9×10⁻⁴ s⁻¹, 8×10⁻⁴ s⁻¹, 7×10⁻⁴ s⁻¹, 6×10⁻⁴ s⁻¹, 5×10⁻⁴ s⁻¹, 4×10⁻⁴ s⁻¹, 3×10⁻⁴ s⁻¹, 2×10⁻⁴ s⁻¹, 1×10⁻⁴ s⁻¹, 9×10⁻⁵ s⁻¹, 8×10⁻⁵ s⁻¹, 7×10⁻⁵ s⁻¹, 6×10⁻⁵ s⁻¹, 5×10⁻⁵ s⁻¹, 4×10⁻⁵ s⁻¹, 3×10⁻⁵ s⁻¹, 2×10⁻⁵ s⁻¹, 1×10⁻⁵ s⁻¹, or less).

Polypeptides, such as single-chain polypeptides, antibodies, antigen-binding fragments thereof, and constructs, described herein may inhibit TNFR2 signaling. In some embodiments, the single-chain polypeptide, antibody, or antigen-binding fragment thereof reduces or inhibits the expression of one or more genes selected from the group consisting of CHUK, NFKBIE, NFKBIA, MAP3K11, TRAF2, TRAF3, relB, and cIAP2/BIRC3. In some embodiments, the single-chain polypeptide, antibody, antigen-binding fragment thereof, or construct inhibits NFKB activation, as assessed, for example, by observing a decrease in the expression of one or more of the above genes or by other methods known in the art for assessing NFKB activation. For instance, antagonistic TNFR2 single-chain polypeptides, antibodies, antigen-binding fragments thereof, and constructs may reduce or inhibit the expression or post-translational modification (e.g., phosphorylation) of one or more of CHUK, NFKBIE, NFKBIA, MAP3K11, TRAF2, TRAF3, relB, or cIAP2/BIRC3, e.g., by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% relative to the expression or post-translational modification (e.g., phosphorylation) of one or more of these molecules isolated from a sample not treated with an antagonistic TNFR2 single-chain polypeptide, antibody, antigen-binding fragment thereof, or construct described herein. Exemplary assays that can be used to determine expression level and phosphorylation state are known in the art and include, e.g., Western blot assays to determine protein content and quantitative reverse transcription polymerase chain reaction (RT-PCR) experiments to determine mRNA content. In preferred embodiments, anti-TNFR2 polypeptides (e.g., single-chain polypeptides, antibodies, antigen-binding fragments thereof, and constructs) are dominant TNFR2 antagonists and are, thus, capable of inhibiting TNFR2 activation even in the presence of a TNFR2 agonist (such as TNFα or Bacillus Calmette-Guérin (BCG)) or a growth-promoting agent, such as IL-2.

Antagonistic TNFR2 polypeptides (e.g., single-chain polypeptides, antibodies, antigen-binding fragments thereof, and constructs) described herein may exhibit one or more, or all, of the following properties:
(a) Suppression of the proliferation of, and/or directly killing of, T-reg cells, for instance, by binding and inactivating TNFR2 on the T-reg cell surface;
(b) Suppression of the proliferation of, and/or directly killing of, MDSCs, for instance, by binding and inactivating TNFR2 on the MDSC surface;
(c) Promotion of the expansion of T effector cells, such as CD8+ T cells; and/or
(d) Suppression of the proliferation of, and/or directly killing of, TNFR2-expressing cancer cells, such as a Hodgkin's lymphoma cell, a cutaneous non-Hodgkin's lymphoma cell, a T cell lymphoma cell, an ovarian cancer cell, a colon cancer cell, a multiple myeloma cell, a renal cell carcinoma cell, a skin cancer cell, a lung cancer cell, a liver cancer cell, an endometrial cancer cell, a hematopoietic or lymphoid cancer cell, a central nervous system cancer cell, a breast cancer cell, a pancreatic cancer cell, a stomach cancer cell, an esophageal cancer cell, and an upper gastrointestinal cancer cell.

For example, antagonistic TNFR2 polypeptides, such as single-chain polypeptides, antibodies, antigen-binding fragments thereof, and constructs, described herein may reduce the total quantity of T-reg or cancer cells in a patient (such as a human patient) or within a sample (e.g., a sample isolated from a patient, such as a human patient undergoing treatment for cancer or an infectious disease as described herein) relative to a patient or sample not treated with the antagonist.

In some embodiments, the antagonistic TNFR2 polypeptide (e.g., a single-chain polypeptide, antibody, or antigen-binding fragment thereof) reduces expression of TNFR2, e.g., by a T-reg cell or a cancer cell (such as a TNFR2+ cancer cell, e.g., a Hodgkin's lymphoma cell, a cutaneous non-Hodgkin's lymphoma cell, a T cell lymphoma cell, an ovarian cancer cell, a colon cancer cell, a multiple myeloma cell, a renal cell carcinoma cell, a skin cancer cell, a lung cancer cell, a liver cancer cell, an endometrial cancer cell, a hematopoietic or lymphoid cancer cell, a central nervous system cancer cell, a breast cancer cell, a pancreatic cancer cell, a stomach cancer cell, an esophageal cancer cell, or an upper gastrointestinal cancer cell), and/or the secretion of soluble TNFR2 by one or more of the foregoing cells.

Antagonistic TNFR2 polypeptides (e.g., single-chain polypeptides, antibodies, antigen-binding fragments thereof, and constructs) described herein may inhibit the proliferation or reduce the total quantity of T-reg cells in a patient (e.g., a human patient) or in a sample (e.g., a sample isolated from a human patient undergoing treatment for cancer or an infectious disease as described herein).

Polypeptides, such as single-chain polypeptides, antibodies, antigen-binding fragments thereof, and constructs, described herein may be capable of reducing or inhibiting the proliferation of, and/or directly killing, T-reg cells and/or cancer cells that express TNFR2. For instance, the cancer cells may be selected from the group consisting of a Hodgkin's lymphoma cell, a cutaneous non-Hodgkin's lymphoma cell, a T cell lymphoma cell, an ovarian cancer cell, a colon cancer cell, a multiple myeloma cell, a renal cell carcinoma cell, a skin cancer cell, a lung cancer cell, a liver cancer cell, an endometrial cancer cell, a hematopoietic or lymphoid cancer cell, a central nervous system cancer cell, a breast cancer cell, a pancreatic cancer cell, a stomach cancer cell, an esophageal cancer cell, or an upper gastrointestinal cancer cell. Binding of TNFR2 on the cancer cell may inhibit or reduce proliferation of the cancer cell and/or may directly kill the cancer cell, such as by promoting apoptosis of the cancer cell.

Antagonistic TNFR2 polypeptides (e.g., single-chain polypeptides, antibodies, antigen-binding fragments thereof, and constructs) described herein bind TNFR2 on the surface of a MDSC (e.g., a cell that expresses all or a subset of proteins and small molecules selected from the group consisting of B7-1 (CD80), B7-H1 (PD-L1), CCR2, CD1d, CD1d1, CD2, CD31 (PECAM-1), CD43, CD44, complement component C5a R1, F4/80 (EMR1), Fcγ RIII (CD16), Fcγ RII (CD32), Fcγ RIIA (CD32a), Fcγ RIIB (CD32b), Fcγ RIIB/C (CD32b/c), Fcγ RIIC (CD32c), Fcγ RIIIA (CD16A), Fcγ RIIIB (CD16b), galectin-3, GP130, Gr-1 (Ly-6G), ICAM-1 (CD54), IL-1 RI, IL-4Rα, IL-6Rα, integrin α4 (CD49d), integrin αL (CD11a), integrin αM (CD11 b), M-CSFR, MGL1 (CD301a), MGL1/2 (CD301a/b), MGL2 (CD301b), nitric oxide, PSGL-1 (CD162), L-selectin (CD62L), siglec-3 (CD33), transferrin receptor (TfR), VEGFR1 (Flt-1), and VEGFR2 (KDR or Flk-1)). Particularly, MDSCs do not express proteins selected from the group consisting of B7-2 (CD86), B7-H4, CD11c, CD14, CD21, CD23 (FcεRII), CD34, CD35, CD40 (TNFRSF5), CD117 (c-kit), HLA-DR, and Sca-1 (Ly6). Binding of TNFR2 on the MDSC may inhibit or reduce proliferation of the MDSC and/or may directly kill the MDSC, such as by promoting apoptosis of the MDSC. Polypeptides, such as single-chain polypeptides, antibodies, antigen-binding fragments thereof, and constructs, described herein may not require TNFα to reduce or inhibit the proliferation of T-reg cells, cancer cells (e.g., TNFR2-expressing cancer cells), and/or MDSCs.

In some embodiments, the polypeptides described herein, such as single-chain polypeptides, antibodies, antigen-binding fragments thereof, and constructs, reduce or inhibit the proliferation of, and/or directly kill, T-reg cells with a greater potency in a patient suffering from cancer relative to a subject that does not have cancer. In some embodiments, the polypeptides described herein, such as single-chain polypeptides, antibodies, antigen-binding fragments thereof, and constructs, reduce or inhibit the proliferation of, and/or directly kill, T-reg cells with a greater potency in the microenvironment of a tumor relative to a site that is free of cancer cells, such as a site distal from a tumor in a patient suffering from cancer or in a subject without cancer.

For example, in some embodiments, the polypeptides described herein, such as single-chain polypeptides, antibodies, antigen-binding fragments thereof, and constructs, reduce or inhibit the proliferation of, and/or directly kill, T-reg cells with a potency that is greater in the microenvironment of a tumor than in a site that is free of cancer cells, such as a site distal from a tumor in a patient suffering from cancer or in a subject without cancer. For instance, the polypeptides described herein, such as single-chain polypeptides, antibodies, antigen-binding fragments thereof, and constructs, may exhibit an IC₅₀ for inhibiting the proliferation of T-reg cells in a tumor microenvironment that is less than the IC₅₀ of the polypeptides for inhibiting the proliferation of T-reg cells in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs, may reduce or inhibit the proliferation of T-reg cells with a potency that is greater in the microenvironment of a tumor containing T cell lymphoma cells (e.g., Hodgkin's or cutaneous non-Hodgkin's lymphoma cells), ovarian cancer cells, colon cancer cells, multiple myeloma cells, or renal cell carcinoma cells than in a site that is free of such cancer cells, such as a site distal from a tumor in a patient suffering from one or more of the foregoing cancers or in a subject without cancer.

In some embodiments, the polypeptides described herein, such as single-chain polypeptides, antibodies, antigen-binding fragments thereof, and constructs, reduce or inhibit the proliferation of, and/or directly kill, MDSCs with a greater potency in a patient suffering from cancer relative to a subject that does not have cancer. In some embodiments, the polypeptides described herein, such as single-chain polypeptides, antibodies, antigen-binding fragments thereof, and constructs, reduce or inhibit the proliferation of, and/or directly kill, MDSCs with a greater potency in the microenvironment of a tumor relative to a site that is free of cancer cells, such as a site distal from a tumor in a patient suffering from cancer or in a subject without cancer.

For example, antagonistic TNFR2 polypeptides (e.g., single-chain polypeptides, antibodies, antigen-binding fragments thereof, and constructs) described herein may bind TNFR2 on the surface of a MDSC present within the microenvironment of a tumor, and may inhibit or reduce proliferation of the MDSC or may promote the apoptosis of the MDSC with a potency that is greater in the microenvironment of a tumor than at a site that is free of cancer cells, such as a site distal from a tumor in a patient suffering from cancer or in a subject without cancer. For instance, the polypeptides described herein, such as single-chain polypeptides, antibodies, antigen-binding fragments thereof, and constructs, may exhibit an IC₅₀ for inhibiting the proliferation of MDSCs in a tumor microenvironment that is less than the IC₅₀ of the polypeptides for inhibiting the proliferation of MDSCs in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs, may reduce or inhibit the proliferation of MDSCs or may promote the apoptosis of MDSCs with a potency that is greater in the microenvironment of a tumor containing T cell lymphoma cells (e.g., Hodgkin's or cutaneous non-Hodgkin's lymphoma cells), ovarian cancer cells, colon cancer cells, multiple myeloma cells, or renal cell carcinoma cells than in a site that is free of such cancer cells, such as a site distal from a tumor in a patient suffering from one or more of the foregoing cancers or in a subject without cancer.

In some embodiments, the polypeptides described herein, such as single-chain polypeptides, antibodies, antigen-binding fragments thereof, and constructs, expand T effector cells, such as CD8+ cytotoxic T cells, with a greater potency in a patient suffering from cancer relative to a subject that does not have cancer. In some embodiments, the polypeptides described herein, such as single-chain polypeptides, antibodies, antigen-binding fragments thereof, and constructs, expand T effector cells, such as CD8+ cytotoxic T cells, with a greater potency in the microenvironment of a tumor relative to a site that is free of cancer cells, such as a site distal from a tumor in a patient suffering from cancer or in a subject without cancer.

For instance, in some embodiments, the polypeptides described herein, such as single-chain polypeptides, antibodies, antigen-binding fragments thereof, and constructs, directly expand T effector cells, such as CD8+ cytotoxic T cells, with a potency that is greater in the microenvironment of a tumor than in a site that is free of cancer cells, such as a site distal from a tumor in a patient suffering from cancer or in a subject without cancer. For instance, the polypeptides described herein may have an EC₅₀ for expanding T effector cells in a cancer patient that is less than the EC₅₀ of the polypeptides for expanding T effector cells in a subject without cancer by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs, may directly expand T effector cells, such as CD8+ cytotoxic T cells, with a potency that is greater in the microenvironment of a tumor containing T cell lymphoma cells (e.g., Hodgkin's or cutaneous non-Hodgkin's lymphoma cells), ovarian cancer cells, colon cancer cells, multiple myeloma cells, or renal cell carcinoma cells than in a site that is free of such cancer cells, such as a site distal from a tumor in a patient suffering from one or more of the foregoing cancers or in a subject without cancer. In some embodiments, the T effector cells (e.g., CD8+ cytotoxic T cells) specifically react with an antigen present on one or more cancer cells, such as Hodgkin's lymphoma cells, cutaneous non-Hodgkin's lymphoma cells, T cell lymphoma cells, ovarian cancer cells, colon cancer cells, multiple myeloma cells, or renal cell carcinoma cells.

A third aspect features a method of identifying a TNFR2 antagonist polypeptide, such as a single-chain polypeptide, antibody, antigen-binding fragment thereof, or construct, by:
(a) exposing a heterogeneous mixture of polypeptides, such as single-chain polypeptides, antibodies, antigen-binding fragments thereof, and constructs, to a peptide having the amino acid sequence of any one of SEQ ID NOs: 31-33, or an amino acid sequence having up to two amino acid substitutions relative to said sequences (e.g., up to two conservative amino acid substitutions relative to said sequences); and
(b) retaining polypeptides, such as single-chain polypeptides, antibodies, antigen-binding fragments thereof, and constructs, that specifically bind the peptide and removing polypeptides, such as single-chain polypeptides, antibodies, antigen-binding fragments thereof, and constructs, that do not specifically bind the peptide, thereby producing an enriched mixture containing at least one TNFR2 antagonist polypeptide, such as a single-chain polypeptide, antibody, antigen-binding fragment thereof, or construct. In some embodiments, the method includes determining the amino acid sequence of one or more of the TNFR2 antagonist polypeptides present in the enriched mixture.

In some embodiments, the peptide is bound to a surface. The polypeptide, such as a single-chain polypeptide, antibody, antigen-binding fragment thereof, or construct, may be expressed on the surface of a viral particle or a cell, such as the surface of a phage, bacterial cell, or yeast cell. The polypeptide may be expressed as one or more polypeptide chains non-covalently bound to ribosomes or covalently bound to mRNA or cDNA. In some embodiments, the peptide having the amino acid sequence of any one of SEQ ID NOs: 31-33 is conjugated to a detectable label, such as a detectable label selected from the group consisting of a fluorescent molecule (e.g., green fluorescent protein, cyan fluorescent protein, yellow fluorescent protein, red fluorescent protein, phycoerythrin, allophycocyanin, hoescht, 4′,6-diamidino-2-phenylindole (DAPI), propidium iodide, fluorescein, coumarin, rhodamine, tetramethylrhoadmine, and cyanine), an epitope tag (e.g., maltose-binding protein, glutathione-S-transferase, a poly-histidine tag, a FLAG-tag, a myc-tag, human influenza hemagglutinin (HA) tag, biotin, and streptavidin), and a radiolabel. In some embodiments, steps (a) and (b) of the method are sequentially repeated one or more times.

A fourth aspect features a method of producing a TNFR2 antagonist antibody or antigen-binding fragment thereof by immunizing a non-human mammal with a peptide containing the sequence of any one of SEQ ID NOs: 31-33, or an amino acid sequence having up to two amino acid substitutions relative to said sequences (e.g., up to two conservative amino acid substitutions relative to said sequences), and collecting serum containing the TNFR2 antagonist antibody or antigen-binding fragment thereof. The non-human mammal may be, for example, selected from the group consisting of a rabbit, mouse, rat, goat, guinea pig, hamster, horse, and sheep.

A fifth aspect features a polypeptide, such as a single-chain polypeptide, antibody, antigen-binding fragment thereof, or construct that is produced by the method of any of the above aspects and embodiments.

Antibodies or antigen-binding fragments thereof described herein may be full-length antibodies or antibody fragments, such as a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv). In some embodiments, the antibody or antigen-binding fragment thereof contains two or more CDRs covalently bound to one another, e.g., by an amide bond, a thioether bond, a carbon-carbon bond, or a disulfide bridge, or by a linker, such as a linker described herein. In some embodiments, the antibody or antigen-binding fragment thereof is a single-chain polypeptide. In some embodiments, the antagonistic TNFR2 polypeptide is a single heavy chain or a single light chain of a full-length antagonistic TNFR2 antibody. In some embodiments, the antibody or antigen-binding fragment thereof has an isotype selected from the group consisting of IgG, IgA, IgM, IgD, and IgE. The antibody or antigen-binding fragment thereof may be conjugated, for example, to a therapeutic agent, such as a cytotoxic agent described herein.

The antagonistic TNFR2 antibody of any of the above aspects can be a bispecific antibody, such as a bispecific monoclonal antibody, in which one arm of the antibody specifically binds TNFR2 and the other specifically binds an immune checkpoint protein, such as PD-1, PD-L1, or CTLA-4, among others described herein. The arm of the bispecific antibody that specifically binds TNFR2 may specifically bind, for example, an epitope of human TNFR2 defined by one or more amino acids within CRD3 and/or an epitope defined by one or more amino acids within CRD4. In some embodiments, the arm of the bispecific antibody that specifically binds TNFR2 specifically binds an epitope of human TNFR2 defined by one or more of amino acids 142-146 (KCRPG) of SEQ ID NO: 1. In some embodiments, the arm of the bispecific antibody that specifically binds TNFR2 does not specifically bind an epitope of human TNFR2 defined by one or more of amino acids 142-146 (KCRPG) of SEQ ID NO: 1. The arm of the bispecific antibody that specifically binds TNFR2 may specifically bind, for example, human TNFR2 at:
(a) a first epitope of TNFR2 within amino acids 174-184 (SSTDICRPHQI), inclusive of the endpoints, of SEQ ID NO: 1;
(b) a second epitope of TNFR2 within amino acids 126-140 (CALSKQEGCRLCAPL), inclusive of the endpoints, of SEQ ID NO: 1; and/or
(c) a third epitope of TNFR2 within amino acids 156-165 (TSDVVCKPCA), inclusive of the endpoints, of SEQ ID NO: 1.

In some embodiments, the bispecific antibody contains one arm that specifically binds TNFR2, such as an epitope of human TNFR2 described above, and one arm that specifically binds an immune checkpoint protein specifically binds PD-1. In some embodiments, the arm of the bispecific antibody that specifically binds PD-1 may specifically bind the same epitope(s) on PD-1 as nivolumab, pembrolizumab, avelumab, durvalumab, or atezolizumab. For example, the arm of the bispecific antibody that specifically binds PD-1 may competitively inhibit the binding of PD-1 to nivolumab, pembrolizumab, avelumab, durvalumab, and/or atezolizumab, as assessed, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds PD-L1. In some embodiments, the arm of the bispecific antibody that specifically binds PD-L1 may specifically bind the same epitope(s) on PD-L1 as atezolizumab or avelumab. For example, the arm of the bispecific antibody that specifically binds PD-L1 may competitively inhibit the binding of PD-L1 to atezolizumab and/or avelumab, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds CTLA-4. In some embodiments, the arm of the bispecific antibody that specifically binds CTLA-4 may specifically bind the same epitope(s) on CTLA-4 as ipilimumab or tremelimumab. For example, the arm of the bispecific antibody that specifically binds CTLA-4 may competitively inhibit the binding of CTLA-4 to ipilimumab and/or tremelimumab, as assessed, for example, using a competitive binding assay described herein or know in the art, such as a competitive ELISA.

Also featured is a polynucleotide encoding a single-chain polypeptide, construct, antibody, or antigen-binding fragment thereof described herein, as well as a vector containing such a polynucleotide. The vector may be an expression vector, such as a eukaryotic expression vector, or a viral vector, such as an adenovirus (Ad, such as serotype 2, 5, 11, 12, 24, 26, 34, 35, 40, 48, 49, 50, 52, or Pan9 adenovirus, or a human, chimpanzee, or rhesus adenovirus), retrovirus (such as a γ-retrovirus or a lentivirus), poxvirus, adeno-associated virus, baculovirus, herpes simplex virus, or a vaccinia virus (such as a modified vaccinia Ankara (MVA). Also featured are host cells, such as prokaryotic and eukaryotic (e.g., mammalian) cells, containing a vector as described herein.

A sixth aspect features a pharmaceutical composition containing an antibody or antigen-binding fragment thereof, single-chain polypeptide, construct, polynucleotide, vector, or host cell as described herein (e.g., a TNFR2 antagonist antibody or antigen-binding fragment thereof) and a pharmaceutically acceptable carrier or excipient. The pharmaceutical composition may contain, for example, an additional therapeutic agent, such as an immunotherapy agent. In some embodiments, the immunotherapy agent is an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, an anti-TWEAKR agent, an anti-TWEAK agent, an anti− cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTp receptor agent, an anti-BCMA agent, an anti-TACI agent, an anti-BAFFR agent, an anti-EDAR2 agent, an anti-TROY agent, or an anti-RELT agent. In some embodiments, the immunotherapy agent is an anti-CTLA4 agent or an anti-PD-1 agent, such as an anti-CTLA4 antibody or antigen-binding fragment thereof (e.g., ipilimumab or tremelimumab) or an anti-PD-1 antibody or antigen-binding fragment thereof (e.g., nivolumab, pembrolizumab, avelumab, durvalumab, or atezolizumab).

For example, the immunotherapy agent may be an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-CD27 antibody or antigen-binding fragment thereof, an anti-CD30 antibody or antigen-binding fragment thereof, an anti-CD40 antibody or antigen-binding fragment thereof, an anti-4-1 BB antibody or antigen-binding fragment thereof, an anti-GITR antibody or antigen-binding fragment thereof, an anti-OX40 antibody or antigen-binding fragment thereof, an anti-TRAILR1 antibody or antigen-binding fragment thereof, an anti-TRAILR2 antibody or antigen-binding fragment thereof, an anti-TWEAKR antibody or antigen-binding fragment thereof, an anti-TWEAK antibody or antigen-binding fragment thereof, an anti− cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTp receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, or an anti-RELT antibody or antigen-binding fragment thereof. In some embodiments, the immunotherapy agent is an anti− cell surface lymphocyte protein antibody or antigen-binding fragment thereof, such as an antibody or antigen-binding fragment thereof that binds one or more of CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64, CD65, CD66, CD67, CD68, CD69, CD70, CD71, CD72, CD73, CD74, CD75, CD76, CD77, CD78, CD79, CD80, CD81, CD82, CD83, CD84, CD85, CD86, CD87, CD88, CD89, CD90, CD91, CD92, CD93, CD94, CD95, CD96, CD97, CD98, CD99, CD100, CD101, CD102, CD103, CD10⁴, CD10⁵, CD106, CD107, CD108, CD109, CD110, CD111, CD112, CD113, CD114, CD115, CD116, CD117, CD118, CD119, CD120, CD121, CD122, CD123, CD124, CD125, CD126, CD127, CD128, CD129, CD130, CD131, CD132, CD133, CD134, CD135, CD136, CD137, CD138, CD139, CD140, CD141, CD142, CD143, CD144, CD145, CD146, CD147, CD148, CD149, CD150, CD151, CD152, CD153, CD154, CD155, CD156, CD157, CD158, CD159, CD160, CD161, CD162, CD163, CD164, CD165, CD166, CD167, CD168, CD169, CD170, CD171, CD172, CD173, CD174, CD175, CD176, CD177, CD178, CD179, CD180, CD181, CD182, CD183, CD184, CD185, CD186, CD187, CD188, CD189, CD190, CD191, CD192, CD193, CD194, CD195, CD196, CD197, CD198, CD199, CD200, CD201, CD202, CD203, CD204, CD205, CD206, CD207, CD208, CD209, CD210, CD211, CD212, CD213, CD214, CD215, CD216, CD217, CD218, CD219, CD220, CD221, CD222, CD223, CD224, CD225, CD226, CD227, CD228, CD229, CD230, CD231, CD232, CD233, CD234, CD235, CD236, CD237, CD238, CD239, CD240, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CD293, CD294, CD295, CD296, CD297, CD298, CD299, CD300, CD301, CD302, CD303, CD304, CD305, CD306, CD307, CD308, CD309, CD310, CD311, CD312, CD313, CD314, CD315, CD316, CD317, CD318, CD319, and/or CD320.

In some embodiments, the immunotherapy agent is an agent (e.g., a polypeptide, antibody, antigen-binding fragment thereof, a single-chain polypeptide, or construct) that binds a chemokine or lymphokine, such as a chemokine or lymphokine involved in tumor growth. For instance, the immunotherapy agent may be an agent (e.g., polypeptide, antibody, antigen-binding fragment thereof, single-chain polypeptide, or construct) that bind and inhibits the activity of one or more, or all, of CXCL1, CXCL2, CXCL3, CXCL8, CCL2 and CCL5. In some embodiments, the immunotherapy agent is an agent (e.g., a polypeptide, antibody, antigen-binding fragment thereof, a single-chain polypeptide, or construct) that binds and inhibits the activity of one or more, or all, of CCL3, CCL4, CCL8, and CCL22.

The immunotherapy agent may be capable of specifically binding one or more of the immunological targets described in Table 1 of Mahoney et al., Cancer Immunotherapy, 14:561-584 (2015), the disclosure of which is incorporated herein by reference in its entirety. For example, the immunotherapy agent may be an agent, such as an antibody or antigen-binding fragment thereof, that specifically binds one or more of OX40L, TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, or neuropilin.

In some embodiments, the immunotherapy agent is Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat.

In some embodiments, the pharmaceutical composition contains a TNFR2 antagonist antibody or antigen-binding fragment thereof and an anti-PD-1 antibody or antigen-binding fragment thereof, such as nivolumab, pembrolizumab, avelumab, durvalumab, or atezolizumab. In some embodiments, the pharmaceutical composition contains a TNFR2 antagonist antibody or antigen-binding fragment thereof and an anti-PDL1 antibody or antigen-binding fragment thereof. In some embodiments, the pharmaceutical composition contains a TNFR2 antagonist antibody or antigen-binding fragment thereof and an anti-CTLA-4 antibody or antigen-binding fragment thereof, such as ipilimumab or tremelimumab.

In some embodiments, the pharmaceutical composition contains a bispecific antibody, such as a bispecific monoclonal antibody, in which one arm of the antibody specifically binds TNFR2 and the other specifically binds an immune checkpoint protein, such as PD-1, PD-L1, or CTLA-4, among others described herein. The arm of the bispecific antibody that specifically binds TNFR2 may specifically bind, for example, an epitope of human TNFR2 defined by one or more amino acids within CRD3 and/or an epitope defined by one or more amino acids within CRD4. In some embodiments, the arm of the bispecific antibody that specifically binds TNFR2 specifically binds an epitope of human TNFR2 defined by one or more of amino acids 142-146 (KCRPG) of SEQ ID NO: 1. In some embodiments, the arm of the bispecific antibody that specifically binds TNFR2 does not specifically bind an epitope of human TNFR2 defined by one or more of amino acids 142-146 (KCRPG) of SEQ ID NO: 1. The arm of the bispecific antibody that specifically binds TNFR2 may specifically bind, for example, human TNFR2 at:
(a) a first epitope of TNFR2 within amino acids 174-184 (SSTDICRPHQI), inclusive of the endpoints, of SEQ ID NO: 1;
(b) a second epitope of TNFR2 within amino acids 126-140 (CALSKQEGCRLCAPL), inclusive of the endpoints, of SEQ ID NO: 1; and/or
(c) a third epitope of TNFR2 within amino acids 156-165 (TSDVVCKPCA), inclusive of the endpoints, of SEQ ID NO: 1.

In some embodiments, the bispecific antibody contains one arm that specifically binds TNFR2, such as an epitope of human TNFR2 described above, and one arm that specifically binds an immune checkpoint protein specifically binds PD-1. In some embodiments, the arm of the bispecific antibody that specifically binds PD-1 may specifically bind the same epitope(s) on PD-1 as nivolumab, pembrolizumab, avelumab, durvalumab, or atezolizumab. For example, the arm of the bispecific antibody that specifically binds PD-1 may competitively inhibit the binding of PD-1 to nivolumab, pembrolizumab, avelumab, durvalumab, and/or atezolizumab, as assessed, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds PD-L1. In some embodiments, the arm of the bispecific antibody that specifically binds PD-L1 may specifically bind the same epitope(s) on PD-L1 as atezolizumab or avelumab. For example, the arm of the bispecific antibody that specifically binds PD-L1 may competitively inhibit the binding of PD-L1 to atezolizumab and/or avelumab, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds CTLA-4. In some embodiments, the arm of the bispecific antibody that specifically binds CTLA-4 may specifically bind the same epitope(s) on CTLA-4 as ipilimumab or tremelimumab. For example, the arm of the bispecific antibody that specifically binds CTLA-4 may competitively inhibit the binding of CTLA-4 to ipilimumab and/or tremelimumab, as assessed, for example, using a competitive binding assay described herein or know in the art, such as a competitive ELISA.

In some embodiments, the additional therapeutic agent in the pharmaceutical composition is a chemotherapeutic agent, such as a chemotherapeutic agent described herein. The antibody or antigen-binding fragment thereof, single-chain polypeptide, construct, polynucleotide, vector, or host cell described herein (e.g., a TNFR2 antagonist antibody or antigen-binding fragment thereof) may be formulated for co-administration with a chemotherapeutic agent, for instance, by admixing the antibody or antigen-binding fragment thereof, single-chain polypeptide, construct, polynucleotide, vector, or host cell with the chemotherapeutic agent. In some embodiments, the antibody or antigen-binding fragment thereof, single-chain polypeptide, construct, polynucleotide, vector, or host cell is formulated for administration separately from the chemotherapeutic agent. In some embodiments, the chemotherapeutic agent is conjugated directly to the antibody or antigen-binding fragment thereof, single-chain polypeptide, construct, polynucleotide, vector, or host cell, for instance, using bond-forming techniques described herein or known in the art.

In some embodiments, the additional therapeutic agent in the pharmaceutical composition is a chimeric antigen receptor (CAR-T) agent, such as a T cell engineered to express a T cell receptor that specifically binds one or more antigens expressed on the surface of a cancer cell. The antibody or antigen-binding fragment thereof, single-chain polypeptide, construct, polynucleotide, vector, or host cell described herein (e.g., a TNFR2 antagonist antibody or antigen-binding fragment thereof) may be formulated for co-administration with a CAR-T agent for instance, by admixing the antibody or antigen-binding fragment thereof, single-chain polypeptide, construct, polynucleotide, vector, or host cell with the CAR-T agent. In some embodiments, the antibody or antigen-binding fragment thereof, single-chain polypeptide, construct, polynucleotide, vector, or host cell is formulated for administration separately from the chemotherapeutic agent, such as by way of serial administration.

In some embodiments, the additional therapeutic agent is a small molecule anti-cancer agent, such as a small molecule described in Imai et al., Nature Reviews Cancer 6:714-727 (2006), the disclosure of which is incorporated herein by reference.

In some embodiments, the additional therapeutic agent is a cancer vaccine, such as a vaccine described in Palucka et al., Journal of Immunology 186:1325-1331 (2011), the disclosure of which is incorporated herein by reference.

Also featured is a method of producing a polypeptide (e.g., single-chain polypeptide, construct, antibody, or antigen-binding fragment) as described herein by expressing a polynucleotide encoding the single-chain polypeptide, construct, antibody, or antigen-binding fragment thereof in a host cell and recovering the single-chain polypeptide, antibody, or antigen-binding fragment thereof from host cell medium.

In another aspect, featured is a method of inhibiting an immune response mediated by a regulatory T cell, as well as a method of treating a cell proliferation disorder in a human, by administering a single-chain polypeptide, construct, antibody, or antigen-binding fragment thereof, polynucleotide, vector, host cell, pharmaceutical composition (e.g., a pharmaceutical composition containing a TNFR2 antagonist antibody or antigen-binding fragment thereof and, optionally, an immunotherapy agent, such as an anti-CTLA4, anti-PD-1, or anti-PDL1 antibody or antigen-binding fragment thereof) and/or immunotherapy agent, optionally in combination with an additional therapy described herein, to the human in need of treatment. In some embodiments, the method includes administering to the patient a TNFR2 antagonist antibody or antigen-binding fragment thereof and an anti-PD-1 antibody or antigen-binding fragment thereof, such as nivolumab, pembrolizumab, avelumab, durvalumab, or atezolizumab. In some embodiments, the method includes administering to the patient a TNFR2 antagonist antibody or antigen-binding fragment thereof and an anti-PDL1 antibody or antigen-binding fragment thereof. In some embodiments, the method includes administering to the patient a TNFR2 antagonist antibody or antigen-binding fragment thereof and an anti-CTLA-4 antibody or antigen-binding fragment thereof, such as ipilimumab or tremelimumab.

In an additional aspect, featured is a composition containing a single-chain polypeptide, construct, antibody, or antigen-binding fragment thereof, polynucleotide, vector, host cell, pharmaceutical composition and/or immunotherapy agent, optionally in combination with an additional therapy described herein, for inhibiting an immune response mediated by a regulatory T cell, as well as for treating a cell proliferation disorder in a human. In some embodiments, the composition contains a TNFR2 antagonist antibody or antigen-binding fragment thereof and an anti-PD-1 antibody or antigen-binding fragment thereof, such as nivolumab, pembrolizumab, avelumab, durvalumab, or atezolizumab. In some embodiments, the composition contains a TNFR2 antagonist antibody or antigen-binding fragment thereof and an anti-PDL1 antibody or antigen-binding fragment thereof. In some embodiments, the composition contains a TNFR2 antagonist antibody or antigen-binding fragment thereof and an anti-CTLA-4 antibody or antigen-binding fragment thereof, such as ipilimumab or tremelimumab.

In some embodiments of either of the preceding two aspects, the antagonistic TNFR2 polypeptide is a full-length antibody. In some embodiments, the antagonistic TNFR2 polypeptide is an antibody fragment that specifically binds TNFR2, such as at one or more epitopes on TNFR2 as described herein. For example, the antagonistic TNFR2 polypeptide may be a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv). In some embodiments, the antibody or antigen-binding fragment thereof contains two or more CDRs covalently bound to one another, e.g., by an amide bond, a thioether bond, a carbon-carbon bond, or a disulfide bridge, or by a linker, such as a linker described herein. In some embodiments, the antibody or antigen-binding fragment thereof is a single-chain polypeptide. In some embodiments, the antagonistic TNFR2 polypeptide is a single heavy chain or a single light chain of a full-length antagonistic TNFR2 antibody. In some embodiments, the antibody or antigen-binding fragment thereof has an isotype selected from the group consisting of IgG, IgA, IgM, IgD, and IgE. The antibody or antigen-binding fragment thereof may be conjugated, for example, to a therapeutic agent, such as a cytotoxic agent described herein.

In some embodiments of either of the preceding two aspects, the antagonistic TNFR2 antibody is a bispecific antibody, such as a bispecific monoclonal antibody, in which one arm of the antibody specifically binds TNFR2 and the other specifically binds an immune checkpoint protein, such as PD-1, PD-L1, or CTLA-4, among others described herein. The arm of the bispecific antibody that specifically binds TNFR2 may specifically bind, for example, an epitope of human TNFR2 defined by one or more amino acids within CRD3 and/or an epitope defined by one or more amino acids within CRD4. In some embodiments, the arm of the bispecific antibody that specifically binds TNFR2 specifically binds an epitope of human TNFR2 defined by one or more of amino acids 142-146 (KCRPG) of SEQ ID NO: 1. In some embodiments, the arm of the bispecific antibody that specifically binds TNFR2 does not specifically bind an epitope of human TNFR2 defined by one or more of amino acids 142-146 (KCRPG) of SEQ ID NO: 1. The arm of the bispecific antibody that specifically binds TNFR2 may specifically bind, for example, human TNFR2 at:
(a) a first epitope of TNFR2 within amino acids 174-184 (SSTDICRPHQI), inclusive of the endpoints, of SEQ ID NO: 1;
(b) a second epitope of TNFR2 within amino acids 126-140 (CALSKQEGCRLCAPL), inclusive of the endpoints, of SEQ ID NO: 1; and/or
(c) a third epitope of TNFR2 within amino acids 156-165 (TSDVVCKPCA), inclusive of the endpoints, of SEQ ID NO: 1.

In some embodiments, the bispecific antibody contains one arm that specifically binds TNFR2, such as an epitope of human TNFR2 described above, and one arm that specifically binds an immune checkpoint protein specifically binds PD-1. In some embodiments, the arm of the bispecific antibody that specifically binds PD-1 may specifically bind the same epitope(s) on PD-1 as nivolumab, pembrolizumab, avelumab, durvalumab, or atezolizumab. For example, the arm of the bispecific antibody that specifically binds PD-1 may competitively inhibit the binding of PD-1 to nivolumab, pembrolizumab, avelumab, durvalumab, and/or atezolizumab, as assessed, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds PD-L1. In some embodiments, the arm of the bispecific antibody that specifically binds PD-L1 may specifically bind the same epitope(s) on PD-L1 as atezolizumab or avelumab. For example, the arm of the bispecific antibody that specifically binds PD-L1 may competitively inhibit the binding of PD-L1 to atezolizumab and/or avelumab, for example, using a competitive binding assay described herein or know in the art, such as a competitive ELISA.

In some embodiments, the bispecific antibody contains one arm that specifically binds TNFR2, such as an epitope of human TNFR2 described above, and one arm that specifically binds CTLA-4. In some embodiments, the arm of the bispecific antibody that specifically binds CTLA-4 may specifically bind the same epitope(s) on CTLA-4 as ipilimumab or tremelimumab. For example, the arm of the bispecific antibody that specifically binds CTLA-4 may competitively inhibit the binding of CTLA-4 to ipilimumab and/or tremelimumab, as assessed, for example, using a competitive binding assay described herein or know in the art, such as a competitive ELISA.

In some embodiments, the single-chain polypeptide, construct, antibody, or antigen-binding fragment thereof, polynucleotide, vector, host cell, or pharmaceutical composition is administered to a patient or formulated for administration to a patient in combination with an immunotherapy agent, such as an immunotherapy agent described herein (e.g., an anti-PD-1 antibody, such as nivolumab, pembrolizumab, avelumab, durvalumab, or atezolizumab; an anti-PD-L1 antibody, such as atezolizumab or avelumab; and/or an anti-CTLA-4 antibody, such as ipilimumab or tremelimumab).

In some embodiments, the additional therapy is a small molecule anti-cancer agent, such as a small molecule described in Imai et al., Nature Reviews Cancer 6:714-727 (2006), the disclosure of which is incorporated herein by reference. In some embodiments, the additional therapy is a cancer vaccine, such as a vaccine described in Palucka et al., Journal of Immunology 186:1325-1331 (2011), the disclosure of which is incorporated herein by reference. In some embodiments, the additional therapy is a CAR-T agent, such as a CAR-T agent that specifically binds a tumor antigen. In some embodiments, the additional therapy is radiation therapy.

In some embodiments, the cell proliferation disorder may be a cancer, such as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer. In particular cases, the cell proliferation disorder may be a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

The cancer may be, for instance, one that is characterized by cells that express TNFR2, such as, for instance, Hodgkin's lymphoma, cutaneous non-Hodgkin's lymphoma, T cell lymphoma, ovarian cancer, colon cancer, multiple myeloma, renal cell carcinoma, skin cancer, lung cancer, liver cancer, endometrial cancer, a hematopoietic or lymphoid cancer, a central nervous system cancer (e.g., glioma, neuroblastoma, and other cancers of central nervous system cells described herein), breast cancer, pancreatic cancer, stomach cancer, esophageal cancer, and upper gastrointestinal cancer, by administration of an antagonistic TNFR2 polypeptide (e.g., single-chain polypeptide, construct, antibody, or antigen-binding fragment thereof), a polynucleotide, vector, or host cell described herein to a patient (e.g., a mammalian patient, such as a human patient). For instance, provided is a method of treating ovarian cancer by administration of an antagonistic TNFR2 antibody or antigen-binding fragment thereof as described herein to a patient (e.g., a mammalian patient, such as a human patient). Also featured is a composition containing a single-chain polypeptide, construct, antibody, or antigen-binding fragment thereof, polynucleotide, vector, or host cell described herein for treating Hodgkin's lymphoma, cutaneous non-Hodgkin's lymphoma, T cell lymphoma, ovarian cancer, colon cancer, multiple myeloma, renal cell carcinoma, skin cancer, lung cancer, liver cancer, endometrial cancer, a hematopoietic or lymphoid cancer, a central nervous system cancer (e.g., glioma, neuroblastoma, and other cancers of central nervous system cells described herein), breast cancer, pancreatic cancer, stomach cancer, esophageal cancer, and upper gastrointestinal cancer in a patient (e.g., a human patient).

Also featured is a method of treating an infectious disease in a patient (e.g., a human patient) by administering a single-chain polypeptide, construct, antibody, or antigen-binding fragment thereof, polynucleotide, vector, host cell, or pharmaceutical composition described herein to the human in need of treatment, as well as a composition containing a single-chain polypeptide, construct, antibody, or antigen-binding fragment thereof, polynucleotide, vector, host cell, or pharmaceutical composition described herein for treating an infectious disease in a patient (e.g., a human patient). In some embodiments, the infectious disease is caused by a virus, a bacterium, a fungus, or a parasite. For instance, viral infections that can be treated according to the methods described herein include hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Parand virus, Pichinde virus, Pirital virus, Sabid virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O‘nyong’nyong virus, and the chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), influenza virus, severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, mumps virus, poliovirus, human enterovirus (A, B, C, and D), hepatitis A virus, coxsackievirus, hepatitis B virus, human papilloma virus, adeno-associated virus, astrovirus, JC virus, BK virus, SV40 virus, Norwalk virus, rotavirus, human immunodeficiency virus (HIV), human T lymphotropic virus Types I and II, and transmissible spongiform encephalopathy, such as chronic wasting disease.

In some embodiments, bacterial infections that can be treated according to the methods described herein include those caused by a bacterium belonging to a genus selected from the group consisting of Salmonella, Streptococcus, Bacillus, Listeria, Corynebacterium, Nocardia, Neisseria, Actinobacter, Moraxella, Enterobacteriacece (e.g., E. coli, such as 01 57:H7), Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus. In addition, parasitic infections that can be treated according to the methods described herein include those caused by Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, or Echinococcus granulosus.

Also featured are kits, such as a kit that contains a single-chain polypeptide, construct, antibody, or antigen-binding fragment described herein (e.g., an antagonist TNFR2 antibody), a polynucleotide described herein, a vector described herein, a host cell, and/or a pharmaceutical composition described herein. In some cases, kits described herein may contain instructions for transfecting a vector described herein into a host cell described herein. Optionally, kits may contain instructions for (and optionally, a reagent that can be used for) expressing a single-chain polypeptide, antibody, or antigen-binding fragment described herein in a host cell described herein. A kit described herein may also contain instructions for administering a single-chain polypeptide, construct, antibody or antigen-binding fragment described herein, a polynucleotide described herein, a vector described herein, a host cell described herein, or a pharmaceutical composition described herein to a human patient. Optionally a kit may contain instructions for making or using an antibody or antigen-binding fragment described herein, a polynucleotide described herein, a vector described herein, a host cell described herein, or a pharmaceutical composition described herein.

### Definitions

As used herein, the term “about” refers to a value that is no more than 10% above or below the value being described. For example, the term “about 5 nM” indicates a range of from 4.5 nM to 5.5 nM.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. Moreover, unless otherwise indicated, the term “monoclonal antibody” (mAb) is meant to include both intact molecules, as well as, antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the terms “anti-tumor necrosis factor receptor 2 antibody,” “TNFR2 antibody,” “anti-TNFR2 antibody portion,” and/or “anti-TNFR2 antibody fragment” and the like include any protein or peptide-containing molecule that includes at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, that is capable of specifically binding to TNFR2. For instance, two or more portions of an immunoglobulin molecule may be covalently bound to one another, e.g., via an amide bond, a thioether bond, a carbon-carbon bond, a disulfide bridge, or by a linker, such as a linker described herein or known in the art. TNFR2 antibodies also include antibody-like protein scaffolds, such as the tenth fibronectin type III domain (¹⁰Fn3), which contains BC, DE, and FG structural loops similar in structure and solvent accessibility to antibody CDRs. The tertiary structure of the ¹⁰Fn3 domain resembles that of the variable region of the IgG heavy chain, and one of skill in the art can graft, e.g., the CDRs of a TNFR2 monoclonal antibody onto the fibronectin scaffold by replacing residues of the BC, DE, and FG loops of ¹⁰Fn3 with residues from the CDR-H1, CDR-H2, or CDR-H3 regions of a TNFR2 monoclonal antibody.

As used herein, the terms “antagonist TNFR2 antibody” and “antagonistic TNFR2 antibody” refer to TNFR2 antibodies that are capable of inhibiting or reducing activation of TNFR2 and/or attenuating one or more signal transduction pathways mediated by TNFR2. For example, antagonistic TNFR2 antibodies can inhibit or reduce the growth and proliferation of regulatory T cells. Antagonistic TNFR2 antibodies may inhibit or reduce TNFR2 activation by blocking TNFR2 from binding TNFα. In this way, antagonistic TNFR2 antibodies may block the trimerization of TNFR2 that would otherwise be induced by interacting with TNFα, thus resulting in suppression of TNFR2 activity.

As used herein, the term “bispecific antibodies” refers to antibodies (e.g., monoclonal, often human or humanized antibodies) that have binding specificities for at least two different antigens. For example, one of the binding specificities can be directed towards TNFR2, the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the phrase “chemotherapeutic agent” refers to any chemical agent with therapeutic usefulness in the treatment of cancer, such as a cancer described herein. Chemotherapeutic agents encompass both chemical and biological agents. These agents can function to inhibit a cellular activity upon which a cancer cell depends for continued survival. Categories of chemotherapeutic agents include alkylating/alkaloid agents, antimetabolites, hormones, hormone analogs, and antineoplastic drugs.

Exemplary chemotherapeutic agents suitable for use in conjunction with the compositions and methods described herein include, without limitation, those set forth in Slapak and Kufe, Principles of Cancer Therapy, Chapter 86 in Harrison's Principles of Internal medicine, 14^{th} edition; Perry et al., Chemotherapeutic, Chapter 17 in Abeloff, Clinical Oncology 2^{nd} ed., 2000; Baltzer L. and Berkery R. (eds): Oncology Pocket Guide to Chemotherapeutic, 2^{nd} ed. St. Luois, mosby-Year Book, 1995; Fischer D. S., Knobf M. F., Durivage H.J. (eds): The Cancer Chemotherapeutic Handbook, 4^{th} ed. St. Luois, Mosby-Year Handbook, the disclosures of each of which are incorporated herein by reference as they pertain to chemotherapeutic agents.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, 1985, Science 229(4719): 1202-7; Oi et al, 1986, BioTechniques 4:214-221; Gillies et al, 1985, J. Immunol. Methods 125:191-202; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may comprising modifications in these hybrid hypervariable positions. The variable domains of native heavy and light chains each comprise four framework regions that primarily adopt a 3-sheet configuration, connected by three CDRs, which form loops that connect, and in some cases form part of, the 3-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “conjugate” refers to a compound formed by the chemical bonding of a reactive functional group of one molecule with an appropriately reactive functional group of another molecule.

As used herein in the context of a TNFR2 antagonist, the term “construct” refers to a fusion protein containing a first polypeptide domain bound to a second polypeptide domain. The polypeptide domains may each independently be antagonistic TNFR2 single chain polypeptides, for instance, as described herein. The first polypeptide domain may be covalently bound to the second polypeptide domain, for instance, by way of a linker, such as a peptide linker or a disulfide bridge, among others. Exemplary linkers that may be used to join the polypeptide domains of an antagonistic TNFR2 construct include, without limitation, those that are described in Leriche et al., Bioorg. Med. Chem., 20:571-582 (2012), the disclosure of which is incorporated herein by reference in its entirety.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structure, peptides configured in this way typically trimerize so as to position the V_{H} and V_{L} domains of neighboring peptide chains spatially proximal to one another to permit proper folding (see Holliger et al., Proc. Natl. Acad. Sci. USA 90:6444-48, 1993; incorporated herein by reference).

As used herein, a “dominant antagonist” of TNFR2 is an antagonist (e.g., an antagonistic polypeptide, such as a single-chain polypeptide, antibody, or antigen-binding fragment thereof) that is capable of inhibiting TNFR2 activation even in the presence of a TNFR2 agonist, such as TNFα, or IL-2. For example, a TNFR2 antagonist is a dominant antagonist if the IC₅₀ of the antagonist increases by less than 200% (e.g., less than 200%, 100%, 50%, 45%, 40%, 35%, 30%, 25%, 20%, 15%, 10%, 5%, 1%, or less) in the presence of a TNFR2 agonist (e.g., TNFα) or IL-2 relative to the IC₅₀ of the antagonist as measured in the same assay in the absence of a TNFR2 agonist, such as TNFα, or IL-2. Inhibition of TNFR2 activation can be assessed, for instance, by measuring the inhibition of proliferation of TNFR2+ cells, such as T-reg cells, cancer cells that express TNFR2, or myeloid-derived suppressor cells, as well as by measuring the inhibition of NFKB signaling (e.g., by monitoring the reduction in expression of one or more genes selected from the group consisting of CHUK, NFKBIE, NFKBIA, MAP3K11, TRAF2, TRAF3, relB, and cIAP2/BIRC3 in a conventional gene expression assay). Cell proliferation assays and gene expression assays that can be used to monitor TNFR2 activation are described herein, for instance, in Examples 9 and 12, respectively.

As used herein, a “dual variable domain immunoglobulin” (“DVD-Ig”) refers to an antibody that combines the target-binding variable domains of two monoclonal antibodies via linkers to create a tetravalent, dual-targeting single agent. (Gu et al., Meth. Enzymol., 502:25-41, 2012; incorporated by reference herein). Suitable linkers for use in the light chains of the DVDs described herein include those identified on Table 2.1 on page 30 of Gu et al.: the short K chain linkers ADAAP (SEQ ID NO: 12) (murine) and TVAAP (SEQ ID NO: 13) (human); the long K chain linkers ADAAPTVSIFP (SEQ ID NO: 14) (murine) and TVAAPSVFIFPP (SEQ ID NO: 15) (human); the short A chain linker QPKAAP (SEQ ID NO: 16) (human); the long A chain linker QPKAAPSVTLFPP (SEQ ID NO: 17) (human); the GS-short linker GGSGG (SEQ ID NO: 18), the GS-medium linker GGSGGGGSG (SEQ ID NO: 19), and the GS-long linker GGSGGGGSGGGGS (SEQ ID NO: 20) (all GS linkers are murine and human). Suitable linkers for use in the heavy chains of the DVDs include those identified on Table 2.1 on page 30 of Gu & Ghayur, 2012, Methods in Enzymology 502:25-41, incorporated by reference herein: the short linkers AKTTAP (SEQ ID NO: 21) (murine) and ASTKGP (SEQ ID NO: 22) (human); the long linkers AKTTAPSVYPLAP (SEQ ID NO: 23) (murine) and ASTKGPSVFPLAP (SEQ ID NO: 24) (human); the GS-short linker GGGGSG (SEQ ID NO: 25), the GS-medium linker GGGGSGGGGS (SEQ ID NO: 26), and the GS-long linker GGGGSGGGGSGGGG (SEQ ID NO: 26) (all GS linkers are murine and human).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “epitope” refers to a portion of an antigen that is recognized and bound by a polypeptide, such as an antibody, antigen-binding fragment thereof, single-chain polypeptide, or construct as described herein. In the context of a protein antigen (such as TNFR2, e.g., human TNFR2 designated by SEQ ID NO: 1 or TNFR2 of a non-human mammal, such as a non-human mammal described herein), an epitope may be a continuous epitope, which is a single, uninterrupted segment of one or more amino acids covalently linked to one another by peptide bonds in which all of the component amino acids bind the polypeptide (e.g., antibody, antigen-binding fragment thereof, single-chain polypeptide, or construct). Exemplary assays for determining the binding of an antagonistic TNFR2 polypeptide to specific amino acids within an antigen are described in Example 1, below. Continuous epitopes may be composed, for instance, of 1, 5, 10, 15, 20, or more amino acids within an antigen, such as a TNFR2 protein described herein (for instance, human TNFR2 designated by SEQ ID NO: 1). For example, a continuous epitope may be composed of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more amino acids within an antigen). Examples of continuous epitopes on TNFR2 that are bound by antagonistic polypeptides (e.g., single-chain polypeptides, antibodies, antigen-binding fragments thereof, and constructs) described herein include one or more continuous residues of, or all residues of, the SSTDICRPHQI motif (SEQ ID NO: 31), one or more continuous residues of, or all residues of, the CALSKQEGCRLCAPL motif (SEQ ID NO: 32), and one or more continuous residues of, or all residues of, the TSDVVCKPCA motif (SEQ ID NO: 33), as well as corresponding regions on TNFR2 proteins of non-human mammals (e.g., bison, cattle, and others described herein). In some embodiments, an epitope may be a discontinuous epitope, which contains two or more segments of amino acids each separated from one another in an antigen's amino acid sequence by one or more intervening amino acid residues. Discontinuous epitopes may be composed, for instance, of 1, 2, 3, 4, 5, 6, 7, 8, 9, 10, or more such segments of amino acid residues, such as one or more (e.g., 1, 2, 3, 4, 5, 6, 7, 8, 9, 10, or more) segments containing amino acids from within one or more of the SSTDICRPHQI motif (SEQ ID NO: 31), the CALSKQEGCRLCAPL motif (SEQ ID NO: 32), and the TSDVVCKPCA motif (SEQ ID NO: 33) within human TNFR2, as well as corresponding regions on TNFR2 proteins of non-human mammals (e.g., bison, cattle, and others described herein). Despite this separation by intervening amino acids, the segments that compose a discontinuous epitope may be, for instance, spatially proximal to one another in the three-dimensional conformation of the antigen. Exemplary discontinuous epitopes on TNFR2 that are bound by antagonistic polypeptides (e.g., single-chain polypeptides, antibodies, antigen-binding fragments thereof, and constructs) described herein include epitopes containing the following elements: (i) one or more residues, or all residues, of the SSTDICRPHQI motif (SEQ ID NO: 31); (ii) one or more residues, or all residues, of the CALSKQEGCRLCAPL motif (SEQ ID NO: 32), and (iii) one or more residues, or all residues, of the TSDVVCKPCA motif (SEQ ID 290). Additional examples of discontinuous epitopes on TNFR2 that are bound by antagonistic polypeptides (e.g., single-chain polypeptides, antibodies, antigen-binding fragments thereof, and constructs) described herein include epitopes containing elements (i) and (ii) above, epitopes containing elements (i) and (iii) above, and epitopes containing elements (ii) and (iii) above.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al, mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625, 126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “hydrophobic side-chain” refers to an amino acid side-chain that exhibits low solubility in water relative due to, e.g., the steric or electronic properties of the chemical moieties present within the side-chain. Examples of amino acids containing hydrophobic side-chains include those containing unsaturated aliphatic hydrocarbons, such as alanine, valine, leucine, isoleucine, proline, and methionine, as well as amino acids containing aromatic ring systems that are electrostatically neutral at physiological pH, such as tryptophan, phenylalanine, and tyrosine.

As used herein, the term “immunotherapy agent” refers to a compound, such as an antibody, antigen-binding fragment thereof, single-chain polypeptide, or construct as described herein, that specifically binds an immune checkpoint protein (e.g., immune checkpoint receptor or ligand) and exerts an antagonistic effect on the receptor or ligand, thereby reducing or inhibiting the signal transduction of the receptor or ligand that would otherwise lead to a downregulation of the immune response. Immunotherapy agents include compounds, such as antibodies, antigen-binding fragments, single-chain polypeptides, and constructs, capable of specifically binding receptors expressed on the surfaces of hematopoietic cells, such as lymphocytes (e.g., T cells), and suppressing the signaling induced by the receptor or ligand that would otherwise lead to tolerance towards an endogenous (“self”) antigen, such as a tumor-associated antigen. Immunotherapy agents may reduce the signaling induced by the receptor or ligand by, for example, 1%, 2%, 3%, 4%, 5%, 10%, 20%, 30%, 40%, 50%, 60%, 70%, 80%, 90%, 95%, 96%, 97%, 98%, 99%, 99.9%, or 100% relative to the signaling induced by the receptor or ligand exhibited in the absence of the immunotherapy agent. Exemplary assays that can be used to measure the extent of receptor or ligand signaling include, for example, enzyme-linked immunosorbant assay (ELISA) techniques to measure protein expression alterations that are associated with a particular signal transduction pathway, as well as polymerase chain reaction (PCR)-based techniques, such as quantitative PCR, reverse-transcription PCR, and real-time PCR experiments useful for determining changes in gene expression associated with a particular signal transduction pathway, among others. Exemplary methods that can be used to determine whether an agent is an “immunotherapy agent” include the assays described in Mahoney et al., Cancer Immunotherapy, 14:561-584 (2015), the disclosure of which is incorporated herein by reference in its entirety. Examples of immunotherapy agents include, e.g., antibodies or antigen-binding fragments thereof that specifically bind one or more of OX40L, TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, and neuropilin. Additional example of immunotherapy agents include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat. Particular examples of immunotherapy agents that may be used in conjunction with the compositions and methods described herein include anti-PD-1 antibodies and antigen-binding fragments thereof, such as nivolumab, pembrolizumab, avelumab, durvalumab, and atezolizumab, as well as anti-PD-L1 antibodies and antigen-binding fragments thereof, such as atezolizumab and avelumab, and anti-CTLA-4 antibodies and antigen-binding fragments thereof, such as ipilimumab or tremelimumab.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Exemplary multi-specific molecules that include Fc regions and into which anti-TNFR2 antibodies or antigen-binding fragments thereof can be incorporated have been reviewed by Kontermann, 2012, mAbs 4(2):182-197, Yazaki et al, 2013, Protein Engineering, Design & Selection 26(3):187-193, and Grote et al, 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “myeloid-derived suppressor cell” or “MDSC” refers to a cell of the immune system that modulates the activity of a variety of effector cells and antigen-presenting cells, such as T cells, NK cells, dendritic cells, and macrophages, among others. Myeloid derived suppressor cells are distinguished by their gene expression profile, and express all or a subset of proteins and small molecules selected from the group consisting of B7-1 (CD80), B7-H1 (PD-L1), CCR2, CD1d, CD1d1, CD2, CD31 (PECAM-1), CD43, CD44, complement component C5a R1, F4/80 (EMR1), Fcγ RIII (CD16), Fcγ RII (CD32), Fcγ RIIA (CD32a), Fcγ RIIB (CD32b), Fcγ RIIB/C (CD32b/c), Fcγ RIIC (CD32c), Fcγ RIIIA (CD16A), Fcγ RIIIB (CD16b), galectin-3, GP130, Gr-1 (Ly-6G), ICAM-1 (CD54), IL-1 RI, IL-4Ra, IL-6Ra, integrin α4 (CD49d), integrin αL (CD11a), integrin αM (CD11b), M-CSFR, MGL1 (CD301a), MGL1/2 (CD301a/b), MGL2 (CD301 b), nitric oxide, PSGL-1 (CD162), L-selectin (CD62L), siglec-3 (CD33), transferrin receptor (TfR), VEGFR1 (Flt-1), and VEGFR2 (KDR or Flk-1). Particularly, MDSCs do not express proteins selected from the group consisting of B7-2 (CD86), B7-H4, CD11c, CD14, CD21, CD23 (FcεRII), CD34, CD35, CD40 (TNFRSF5), CD117 (c-kit), HLA-DR, and Sca-1 (Ly6).

As used herein, the terms “neutral TNFR2 polypeptide” and “phenotype-neutral TNFR2 polypeptide” refer to a polypeptide (such as a single-chain polypeptide, an antibody, or an antibody fragment) that binds TNFR2 and does not exert an antagonistic or an agonistic effect on TNFR2 activation. For instance, a TNFR2 polypeptide is a neutral TNFR2 polypeptide if the polypeptide binds TNFR2 and neither potentiates nor suppresses TNFR2 activation, for instance, as assessed by measuring the proliferation of TNFR2-expressing cells (e.g., T-reg cells, TNFR2+ cancer cells, and/or MDSCs) and/or by measuring the expression of one or more NFKB target genes, such as CHUK, NFKBIE, NFKBIA, MAP3K11, TRAF2, TRAF3, relB, and/or cIAP2/BIRC3. Exemplary assays for measuring cell proliferation and gene expression are described, e.g., in Examples 9 and 12, respectively.

As used herein, the term “non-native constant region” refers to an antibody constant region that is derived from a source that is different from the antibody variable region or that is a human-generated synthetic polypeptide having an amino sequence that is different from the native antibody constant region sequence. For instance, an antibody containing a non-native constant region may have a variable region derived from a non-human source (e.g., a mouse, rat, or rabbit) and a constant region derived from a human source (e.g., a human antibody constant region), or a constant region derived from another primate, pig, goat, rabbit, hamster, cat, dog, guinea pig, member of the bovidae family (such as cattle, bison, buffalo, elk, and yaks, among others), cow, sheep, horse, or bison, among others).

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a drug over time following administration of the drug to a patient.

As used herein, a “recessive antagonist” of TNFR2 is an antagonist (e.g., an antagonistic polypeptide, such as a single-chain polypeptide, antibody, or antigen-binding fragment thereof) that inhibits TNFR2 activation to a significantly lesser extent in the presence of a TNFR2 agonist, such as TNFα, or IL-2 relative to the extent of inhibition of the same antagonist as measured in the absence of a TNFR2 agonist, such as TNFα, or IL-2. For example, a TNFR2 antagonist is a recessive antagonist if the IC₅₀ of the antagonist increases by, e.g., 10-fold, 20-fold, 30-fold, 40-fold, 50-fold, 60-fold, 70-fold, 80-fold, 90-fold, 100-fold, or more in the presence of a TNFR2 agonist (e.g., TNFα or Bacillus Calmette-Guérin (BCG)) or IL-2 relative to the IC₅₀ of the antagonist as measured in the same assay the absence of a TNFR2 agonist, such as TNFα, or IL-2. Inhibition of TNFR2 activation can be assessed, for instance, by measuring the inhibition of proliferation of TNFR2+ cells, such as T-reg cells, cancer cells that express TNFR2, or myeloid-derived suppressor cells, as well as by measuring the inhibition of NFKB signaling (e.g., by monitoring the reduction in expression of one or more genes selected from the group consisting of CHUK, NFKBIE, NFKBIA, MAP3K11, TRAF2, TRAF3, relB, and cIAP2/BIRC3 in a conventional gene expression assay). Cell proliferation assays and gene expression assays that can be used to monitor TNFR2 activation are described herein, for instance, in Examples 9 and 12, respectively.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, C A, 1990); incorporated herein by reference.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The V_{L} and V_{H} domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 pM, 100 pM, 500 pM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment for a particular disease or condition as described herein (such as cancer or an infectious disease). Examples of subjects and patients include mammals, such as humans, primates, pigs, goats, rabbits, hamsters, cats, dogs, guinea pigs, members of the bovidae family (such as cattle, bison, buffalo, elk, and yaks, among others), cows, sheep, horses, and bison, among others, receiving treatment for diseases or conditions, for example, cell proliferation disorders, such as cancer or infectious diseases.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder, such as the progression of a cell proliferation disorder, such as cancer, or an infectious disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

As used herein, the term “tumor microenvironment” refers to cancer cells that form a tumor and the population of non-cancer cells, molecules, and/or blood vessels within the tumor or that border or surround the cancer cells.

As used herein, the terms “tumor necrosis factor receptor superfamily,” “TNFR superfamily,” or “TNFRS” refer to a group of type I transmembrane proteins with a carboxy-terminal intracellular domain and an amino-terminal extracellular domain characterized by a common cysteine rich domain (CRD). The TNFR superfamily includes receptors that mediate cellular signaling as a consequence of binding to one or more ligands in the TNF superfamily. The TNFR superfamily can be divided into two subgroups: receptors containing the intracellular death domain and those lacking this domain. The death domain is an 80 amino acid motif that propagates apoptotic signal transduction cascades following receptor activation. Exemplary TNFR super family members that contain the intracellular death domain include TNFR1, while TNFR2 represents a TNFR super family protein that does not contain this domain. Members of the TNFR superfamily include TNFR1, TNFR2, RANK, CD30, CD40, Lymphotoxin beta receptor (LT-pR), OX40, Fas receptor, Decoy receptor 3 (DCR3), CD27, 4-11BB, Death receptor 4 (DR4), Death receptor 5 (DR5), Decoy receptor 1 (DCR1), Decoy receptor 2 (DCR2), Osteoprotegrin, TWEAK receptor, TACI, BAFF receptor, Herpesvirus entry mediator, Nerve growth factor receptor, B cell maturation antigen, Glucocorticoid-induced TNFR-related, TROY, Death receptor 6 (DR6), Death receptor 3 (DR3), and Ectodysplasin A2 receptor.

As used herein the term “variable region CDR” includes amino acids in a CDR or complementarity determining region as identified using sequence or structure based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. In certain embodiments, the term “CDR” is a CDR as defined by Kabat based on sequence comparisons.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “V_{H}” refers to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (V_{H}) followed by a number of constant domains. Each light chain of a native antibody has a variable domain at the amino terminus (V_{L}) and a constant domain at the carboxy terminus.

### BRIEF DESCRIPTION OF THE FIGURES

FIG. 1 shows the amino acid sequence of human TNFR2 (SEQ ID NO: 1). Human TNFR2 is numbered herein starting with an N-terminal methionine at position 1 and concluding with a C-terminal serine at position 461 (SEQ ID NO: 1). All references to amino acid positions within TNFR2 are made in the context of the TNFR2 numbering scheme shown in FIG. 1. Shaded residues SSTDICRPHQI (SEQ ID NO: 31), CALSKQEGCRLCAPL (SEQ ID NO: 32), and TSDVVCKPCA (SEQ ID NO: 33) define a discontinuous epitope that is specifically bound by the antagonistic TNFR2 antibody TNFRAB4. Though these residues are not all consecutive in primary sequence, they are spatially proximal in the three dimensional tertiary structure of TNFR2, and are positioned for interaction with an antagonistic TNFR2 antibody described herein. The selective binding of residues within these regions without binding underlined residues KCSPG (SEQ ID NO: 5) promotes antagonism of TNFR2 signaling even without specific binding of one or more of resides KCRPG (SEQ ID NO: 9) of human TNFR2 and equivalent regions within TNFR2 of non-humans, such as non-human mammals.

FIG. 2A-2C are structural models showing the three-dimensional orientation of TNFR2 and epitopes within this receptor that are bound by the antagonistic TNFR2 antibody TNFRAB4. FIG. 2A shows the structure of a TNFR2 monomer and illustrates the spatial orientation of the epitopes, identified in FIG. 1, that are bound by TNFRAB4. FIG. 2B is a structural model showing TNFR2 in an inactivated, anti-parallel dimer conformation. The model shows two monomeric TNFR2 proteins: one on the left of the model and one on the right. The monomer on the right of the figure illustrates the locations of the three epitopes that are specifically bound by monoclonal antibody TNFRAB4, which are represented in shaded ovals. FIG. 2C is a structural model showing TNFR2 in an activated, trimeric conformation. The model shows three monomeric TNFR2 proteins: one on the left of the model, one on the front, right of the model, and one in the rear of the model. The monomer on the front, right side of the figure illustrates the locations of the three epitopes that are specifically bound by monoclonal antibody TNFRAB4, which are represented in shaded ovals.

FIGS. 3A and 3B are graphs showing the effect of TNFR2 monoclonal antibodies on the viability of T-reg cells isolated from healthy human subjects. FIG. 3A demonstrates the ability of an antagonistic TNFR2 antibody that binds epitopes within both CRD3 and CRD4 of TNFR2 to kill T-reg cells in a dose-dependent manner. FIG. 3B shows the effect of a monoclonal TNFR2 antagonist antibody that only binds epitopes within CRD2 and CRD3 of TNFR2 on T-reg cell viability. Values on the y-axis of each figure denote the concentration of the TNFR2 antagonist antibody, in units of pg/ml.

FIG. 4 is a graph showing the expression of TNFR2 by cells of various types. The immunophenotypes of over 500 human cell lines were analyzed, and cells were binned by organ and plotted along the x-axis. Values along the y-axis represent the degree of expression of TNFR2 by cells of each class.

FIGS. 5A-5C are graphs demonstrating the effects of antagonistic TNFR2 antibodies that bind epitopes within both CRD3 and CRD4 of TNFR2 on various TNFR2-expressing cancer cells. FIG. 5A shows the effect of antagonistic TNFR2 antibodies that bind epitopes within both CRD3 and CRD4 of TNFR2 on TNFR2+ human SW480 colon cancer cells. FIG. 5B shows the effect of antagonistic TNFR2 antibodies that bind epitopes within both CRD3 and CRD4 of TNFR2 on TNFR2+ human cutaneous T cell lymphoma cells. FIG. 5C shows the effect of antagonistic TNFR2 antibodies that bind epitopes within both CRD3 and CRD4 of TNFR2 on TNFR2+ human ovarian cancer cells. In each figure, numerical values along the x-axis represent the concentration of the TNFR2 antagonist antibody, in units of pg/ml.

FIG. 6 is a sequence alignment showing portions of the amino acid sequences of TNFR2 from human (amino acid residues 1-386 of SEQ ID NO: 1) and several non-human mammals: cattle (amino acid residues 1-385 of SEQ ID NO: 34), bison (amino acid residues 1-384 of SEQ ID NO: 28), mouse (amino acid residues 1-388 of SEQ ID NO: 29), and rat (amino acid residues 1-388 of SEQ ID NO: 30). Shown in gray shading are residues within human TNFR2 that are specifically bound by antagonistic TNFR2 polypeptides described herein (e.g., the SSTDICRPHQI sequence (SEQ ID NO: 31), the CALSKQEGCRLCAPL sequence (SEQ ID NO: 32), and the TSDVVCKPCA sequence (SEQ ID NO: 33) of human TNFR2), as well as equivalent regions within TNFR2 of cattle, bison, mouse, and rat.

FIGS. 7A-7E are graphs showing the effect of an antagonist TNFR2 antibody and an anti-PD-1 antibody, either alone or in combination, on tumor volume in MC38 murine models of colon cancer. Tumor volume was monitored over a period of up to about 25 days following administration of placebo or the indicated antibody. The individual time course lines in each of FIGS. 7A-7D (marked with shaded and unshaded triangles, circles, and squares) represent tumor volume for each individual mouse tested. FIG. 7A shows the effect of placebo on tumor volume in MC38 mice. FIG. 7B shows the effect of anti-PD-1 antibody treatment on tumor volume in MC38 mice. A p-value of 0.005 was calculated for anti-PD-1 antibody treatment relative to placebo treatment. FIG. 7C shows the effect of antagonist TNFR2 antibody treatment on tumor volume in MC38 mice. A p-value of 0.04 was calculated for antagonist TNFR2 antibody treatment relative to placebo treatment. FIG. 7D shows the effect of combined treatment with an anti-PD-1 antibody and an antagonist TNFR2 antibody on tumor volume in MC38 mice. A p-value of 0.004 was calculated for anti-PD-1 antibody and antagonist TNFR2 antibody treatment relative to placebo treatment. FIG. 7E shows a comparison of the mean changes in tumor volume in MC38 colon cancer mice treated with vehicle, an anti-PD-1 antibody (p=0.0022 relative to vehicle), an antagonist TNR2 antibody (p=0.20 relative to vehicle), and both an anti-PD-1 antibody and an antagonist TNFR2 antibody (p=0.0003 relative to vehicle).

FIGS. 8A-8C are graphs comparing the effect of an antagonist TNFR2 antibody, an anti-PD-1 antibody, and an anti-CTLA-4 antibody, either alone or in the combinations shown, on survival of MC38 murine models of colon cancer. FIG. 8A shows the effect of vehicle, an anti-PD-1 antibody, an antagonist TNFR2 antibody, and the combination of an anti-PD-1 antibody and an antagonist TNFR2 antibody on survival of MC38 mice. FIG. 8B shows the effect of vehicle, an anti-PD-1 antibody, an anti-CTLA-4 antibody, and the combination of an anti-PD-1 antibody and an anti-CTLA-4 antibody on survival of MC38 mice. The data used to generate this graph were obtained from Selby et al., PLoS One 11:e0161779, 2016, the disclosure of which is incorporated herein by reference in its entirety. FIG. 8C shows the effect of vehicle, an anti-PD-1 antibody, and an anti-CTLA-4 antibody on survival of MC38 mice. The data used to generate this graph were obtained from Mosely et al., Cancer Immunology Research 5:0F1-13, 2017, the disclosure of which is incorporated herein by reference in its entirety.

FIGS. 9A-9E are graphs showing the effect of an antagonist TNFR2 antibody and an anti-PD-1 antibody, either alone or in combination, on tumor volume in CT26 murine models of colon cancer. Tumor volume was monitored over a period of up to about 21 days following administration of placebo or the indicated antibody. The individual time course lines in each of FIGS. 9A-9D represent tumor volume for each mouse tested. FIG. 9A shows the effect of placebo on tumor volume in CT26 mice. FIG. 9B shows the effect of anti-PD-1 antibody treatment on tumor volume in CT26 mice. A p-value of 0.002 was calculated for anti-PD-1 antibody treatment relative to placebo treatment. FIG. 9C shows the effect of antagonist TNFR2 antibody treatment on tumor volume in CT26 mice. A p-value of less than 0.0001 was calculated for antagonist TNFR2 antibody treatment relative to placebo treatment. FIG. 9D shows the effect of combined treatment with an anti-PD-1 antibody and an antagonist TNFR2 antibody on tumor volume in CT26 mice. A p-value of 0.004 was calculated for anti-PD-1 antibody and antagonist TNFR2 antibody treatment relative to placebo treatment. FIG. 9E shows a comparison of the mean changes in tumor volume in CT26 colon cancer mice treated with vehicle, an anti-PD-1 antibody, an antagonist TNR2 antibody, and both an anti-PD-1 antibody and an antagonist TNFR2 antibody.

FIGS. 10A-10C are graphs comparing the effect of an antagonist TNFR2 antibody, an anti-PD-1 antibody, and an anti-CTLA-4 antibody, either alone or in the combinations shown, on survival of CT26 murine models of colon cancer. FIG. 10A shows the effect of vehicle, an anti-PD-1 antibody, an antagonist TNFR2 antibody, and the combination of an anti-PD-1 antibody and an antagonist TNFR2 antibody on survival of CT26 mice. FIG. 10B shows the effect of vehicle, an anti-PD-1 antibody, an anti-CTLA-4 antibody, and the combination of an anti-PD-1 antibody and an anti-CTLA-4 antibody on survival of CT26 mice. The data used to generate this graph were obtained from Selby et al., PLoS One 11:e0161779, 2016, the disclosure of which is incorporated herein by reference in its entirety. FIG. 10C shows the effect of vehicle, an anti-PD-1 antibody, and an anti-CTLA-4 antibody on survival of CT26 mice. The data used to generate this graph were obtained from Mosely et al., Cancer Immunology Research 5:OF1-13, 2017, the disclosure of which is incorporated herein by reference in its entirety.

FIGS. 11A and 11B are graphs comparing the effect of an antagonist TNFR2 antibody, an anti-PD-1 antibody, and an anti-CTLA-4 antibody, either alone or in the combinations shown, on Treg survival in CT26 murine models of colon cancer. FIG. 11A shows the effect of vehicle, an anti-PD-1 antibody, an antagonist TNFR2 antibody, and the combination of an anti-PD-1 antibody and an antagonist TNFR2 antibody on Treg survival in CT26 mice. FIG. 11B shows the effect of vehicle, an anti-PD-1 antibody, an anti-CTLA-4 antibody, and the combination of an anti-PD-1 antibody and an anti-CTLA-4 antibody on Treg survival in CT26 mice. The data used to generate this graph were obtained from Selby et al., PLoS One 11:e0161779, 2016, the disclosure of which is incorporated herein by reference in its entirety.

### DETAILED DESCRIPTION

Antagonistic TNFR2 polypeptides described herein, such as single-chain polypeptides, antibodies, antigen-binding fragments thereof, and constructs, inhibit the activation of TNFR2 on TNFR2-expressing cells. This is effectuated, for instance, by binding TNFR2 (e.g., on the exterior surface of a T-reg cell, a cancer cell that expresses TNFR2, or a myeloid-derived suppressor cell (MDSC)) and preventing the receptor from adopting a three-dimensional conformation that is suitable for binding its cognate ligand, TNFα. TNFα potentiates TNFR2 signaling by nucleating a trimer of TNFR2 proteins. It is this trimerization event that brings individual TNFR2 proteins into close proximity and initiates signaling via the MAPK/NFKB/TRAF2/3 pathway, which ultimately leads to cell growth and escape from apoptosis. Antagonistic TNFR2 polypeptides described herein can antagonize this interaction, for instance, by binding the receptor and preventing receptor trimerization. For instance, one mechanism by which this may occur is through the formation of an anti-parallel TNFR2 dimer, which is an inactive structural form of the receptor.

The invention is based in part on the discovery of epitopes within TNFR2 that promote receptor antagonism and various advantageous downstream biological activities. Human TNFR2 contains four cysteine-rich domains (CRDs): CRD1 (amino acid residues 48-76 of SEQ ID NO: 1), CRD2 (amino acid residues 78-120 of SEQ ID NO: 1), CRD3 (amino acid residues 121-162 of SEQ ID NO: 1), and CRD4 (amino acid residues 162-202 of SEQ ID NO: 1). It has been discovered that antagonistic TNFR2 polypeptides (e.g., antibodies, antigen-binding fragments, thereof, single-chain polypeptides, and constructs) that bind one or more epitopes within CRD3 of TNFR2 and/or one or more epitopes within CRD4 of TNFR2 can engender one or more, or all, of the following beneficial activities:
(a) Suppression of the proliferation of, and/or the direct killing of, T-reg cells, for instance, by binding and inactivating TNFR2 on the T-reg cell surface;
(b) Suppression of the proliferation of, and/or the direct killing of, MDSCs, for instance, by binding and inactivating TNFR2 on the MDSC surface;
(c) Promotion of the expansion of T effector cells, such as CD8+ T cells; and/or
(d) Suppression of the proliferation of, and/or the direct killing of, TNFR2-expressing cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells.

Further, it has been discovered that antagonistic TNFR2 polypeptides (e.g., antibodies, antigen-binding fragments, thereof, single-chain polypeptides, and constructs) can antagonize TNFR2 and effectuate one or more, or all, of the activities set forth in (a) through (d), above, by binding one or more epitopes within CRD3 of TNFR2 and/or one or more epitopes within CRD4 of TNFR2 without the need to bind an epitope within the KCRPG sequence (SEQ ID NO: 9) of human TNFR2 or an equivalent epitope in TNFR2 of a non-human primate (e.g., bison or cattle, as described herein). Antagonistic polypeptides (e.g., antibodies, antigen-binding fragments, thereof, single-chain polypeptides, and constructs) described herein, thus, include those that bind TNFR2 exclusively within one or more epitopes of CRD3 and/or one or more epitopes of CRD4, such as those that bind one or more epitopes within CRD3 of TNFR2 and/or one or more epitopes within CRD4 of TNFR2 and that do not bind to an epitope within amino acids 142-146 (KCRPG) of SEQ ID NO: 1 or an equivalent epitope in TNFR2 of a non-human primate (e.g., bison or cattle, as described herein).

It has been discovered that binding of distinct epitopes within CRD3 and CRD4 of TNFR2 promote receptor antagonism, such as epitopes containing one or more continuous or discontinuous residues, or all residues, of the SSTDICRPHQI motif (SEQ ID NO: 31) within TNFR2. Additional epitopes that have been presently been discovered as epitopes that confer antagonistic activity include epitopes that contain one or more continuous or discontinuous residues, or all residues, of the CALSKQEGCRLCAPL motif (SEQ ID NO: 32) within TNFR2, and epitopes containing one or more continuous or discontinuous residues, or all residues, of the TSDVVCKPCA motif (SEQ ID NO: 33) within TNFR2. Particularly, it has been discovered that polypeptides (e.g., single-chain polypeptides, antibodies, antigen-binding fragments thereof, and constructs) that bind one or more, or all, of the above epitopes exhibit one or more, or all, of the beneficial properties set forth in (a) through (d), above.

In some embodiments, antagonistic TNFR2 polypeptides (e.g., single-chain polypeptides, antibodies, antigen-binding fragments thereof, and constructs) described herein exert one or more, or all, of characteristics (a) through (d), above, with a greater potency in the microenvironment of a tumor than in a site that is free of cancer cells. For instance, antagonistic TNFR2 polypeptides (e.g., single-chain polypeptides, antibodies, antigen-binding fragments thereof, and constructs) described herein may exert one or more, or all, of properties (a), (b), and (c), above, preferentially in a patient (such as a mammalian patient, e.g., a human) suffering from cancer relative to a subject (such as a mammalian subject, e.g., a human) that does not have cancer.

The sections that follow provide a description of exemplary characteristics of antagonistic TNFR2 polypeptides described herein, such as single-chain polypeptides, antibodies, antigen-binding fragments thereof, and constructs, as well as their use in therapeutic methods.

### Antagonistic TNFR2 Polypeptides

### Effects on TNFR2/MAPK/TRAF2/3 Signal Transduction Cascades

Anti-TFNR2 polypeptides (e.g., single-chain polypeptides, antibodies, and antigen-binding fragments thereof) described herein are capable of interacting with and inhibiting the activity of TNFR2. Thus, the anti-TNFR2 polypeptides described herein can selectively antagonize the TNFα-TNFR2 interaction rather than promote TNFR2 signaling. This is particularly important for therapeutic applications, such as cancer immunotherapy, as TNFR2 activation upon association with TNFα leads to propagation of the MAPK and TRAF2/3 signal cascade and activation of NFKB-mediated transcription of genes involved in T-reg cell growth and escape from apoptosis (Faustman, et al., Nat. Rev. Drug Disc., 9:482-493, 2010). The TNFR2 polypeptides (e.g., single-chain polypeptides, antibodies, and antigen-binding fragments thereof) described herein bind TNFR2 at one or more specific epitopes that prevent the receptor from forming a trimer with neighboring TNFR2 proteins. This trimerization activates intracellular signaling by TNFR2, which, e.g., promotes proliferation of TNFR2+ cells, such as T-reg cells, MDSCs, and/or TNFR2+ cancer cells. Advantageously, the TNFR2 antagonist polypeptides described herein bind TNFR2 at particular epitopes so as to stabilize TNFR2 in an anti-parallel dimer conformation, in which TNFα binding sites are sterically inaccessible. This prevents TNFα from nucleating TNFR2 trimer formation, which triggers TNFR2 signal transduction. The polypeptides described herein can therefore be used to suppress the growth and proliferation of TNFR2+ cells, such as T-reg cells, MDSCs, and TNFR2+ cancer cells. The suppression of T-reg and MDSC proliferation, for instance, enables the proliferation of T effector cells that can mount an immune response against, e.g., a cancer cell or foreign pathogen. Thus, antagonistic TNFR2 polypeptides described herein can be administered to a mammalian subject, such as a human patient, with a cell proliferation disorder or an infectious disease, in order to enhance the effectiveness of an immune response (e.g., an immune response against cancer cells or pathogenic organisms) in the patient.

### Effects on T-Reg Cell Proliferation

Antagonistic TNFR2 polypeptides, such as single-chain polypeptides, antibodies, or antigen-binding fragments thereof described herein can be used to attenuate the activity of T-reg cells that typically accompanies T cell-mediated cytotoxicity against self cells, such as the attack of a tumor cell by a T lymphocyte. This can be achieved, for instance, due to the ability of antagonistic TNFR2 polypeptides described herein to inhibit the proliferation of, and/or to directly kill, T-reg cells. Antagonistic TNFR2 polypeptides can, thus, be administered to a mammalian subject, such as a human (e.g., by any of a variety of routes of administration described herein) in order to prolong the duration of an adaptive immune response, such as a response against a cancer cell or a pathogenic organism. In this way, for example, antagonistic TNFR2 polypeptides, such as single-chain polypeptides, antibodies, or antigen-binding fragments thereof described herein, may synergize with existing techniques to enhance T lymphocyte-based therapy for cancer and for infectious diseases. For instance, TNFR2 antagonists described herein may be administered to suppress T-reg cell activity, thereby enhancing the cytotoxic effect of tumor reactive T cells. TNFR2 antagonists may also synergize with existing strategies to promote tumor-reactive T cell survival, such as lymphodepletion and growth factor therapy, and in turn prolong the duration of anti-tumor reactivity in vivo.

Antagonistic TNFR2 polypeptides, such as single-chain polypeptides, antibodies, and antigen-binding fragments thereof can also be used to treat a broad array of infectious diseases in a mammalian subject (e.g., a human), as inhibition of T-reg proliferation promotes the activity of CD8+ T lymphocytes capable of mounting an attack on pathogenic organisms. Additionally, antagonistic TNFR2 antibodies and antigen-binding fragments thereof described herein can be used to treat a wide variety of infectious diseases, such as Mycobacterium tuberculosis, in a human or an agricultural farm animal (e.g., a bovine mammal, pig, cow, horse, sheep, goat, cat, dog, rabbit, hamster, guinea pig, or other non-human mammal).

### Direct Effects on TNFR2+ Cancer Cells

Antagonistic TNFR2 polypeptides, such as single-chain polypeptides, antibodies, or antigen-binding fragments thereof described herein may bind and inactivate TNFR2 on the surface of a cancer cell, such as a TNFR2+ tumor cell. For instance, antagonistic TNFR2 antibodies and antigen-binding fragments thereof described herein may bind TNFR2 on the surface a T cell lymphoma cell (e.g., a Hodgkin's or cutaneous non-Hodgkin's lymphoma cell), ovarian cancer cell, colon cancer cell, multiple myeloma cell, or renal cell carcinoma cell, among others. The ability of antagonistic TNFR2 antibodies and antigen-binding fragments thereof described herein to bind TNFR2 directly on a cancer cell provides another pathway by which these molecules may attenuate cancer cell survival and proliferation. For instance, an antagonistic TNFR2 polypeptide described herein, such as an antagonistic TNFR2 single-chain polypeptide, antibody, antigen-binding fragment thereof, or construct, may bind TNFR2 directly on the surface of a cancer cell (e.g., a cutaneous T cell lymphoma cell, ovarian cancer cell, colon cancer cell, or multiple myeloma cell, such as an ovarian cancer cell) in order to suppress the ability of the cell to proliferate and/or to promote apoptosis of the cell.

### TNFR2 Antagonist Polypeptides are not Reliant on Additional TNFR2-Binding Agents for Activity

Significantly, antagonistic TNFR2 polypeptides, such as single-chain polypeptides, antibodies, or antigen-binding fragments thereof described herein are capable of binding TNFR2 and suppressing TNFR2-mediated signalling without the need for an endogenous TNFR2-binding agent, such as TNFα. Antagonistic TNFR2 polypeptides, such as single-chain polypeptides, antibodies, and antigen-binding fragments thereof described herein do not require TNFα to attenuate T-reg and/or cancer cell proliferation. Without being limited by mechanism, antagonistic TNFR2 antibodies or antigen-binding fragments thereof described herein may exhibit this property due to the ability of these antibodies or antigen-binding fragments thereof to bind TNFR2 at particular epitopes that, when bound, stabilize the anti-parallel dimer conformation of this receptor. This structural configuration is not capable of potentiating NFKB signaling. By maintaining TNFR2 in an inactive structural state, antagonistic TNFR2 polypeptides described herein may prevent TNFR2 agonists from restoring cell growth and/or may result in the direct killing (e.g., by apoptosis) of a TNFR2+ cell, such as a T-reg cell, MDSC, or TNFR2+ cancer cell).

For instance, antagonistic TNFR2 polypeptides, such as single-chain polypeptides, antibodies, antigen-binding fragments thereof, and constructs, described herein may bind TNFR2 on the surface of a TNFR2+ cell, such as a T-reg cell, cancer cell, or myeloid-derived suppressor cell (MDSC) and inhibit the proliferation of such cells in the presence or absence of TNFα. For example, antagonistic TNFR2 polypeptides, such as single-chain polypeptides, antibodies, and antigen-binding fragments thereof described herein may inhibit the proliferation of such cells by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to such cells that are not treated with the TNFR2 antagonist polypeptide. The antagonistic TNFR2 polypeptide (e.g., single-chain polypeptide, antibody, or antigen-biding fragment thereof) may exhibit an IC₅₀ value in such a cell proliferation assay that is largely unchanged by the presence or absence of TNFα (e.g., an IC₅₀ value in the presence of TNFα that is changed by less than 50%, 45%, 40%, 35%, 25%, 20%, 15%, 10%, 9%, 8%, 7%, 6%, 5%, 4%, 3%, 2%, or less than 1% relative to the IC₅₀ value of the antagonistic TNFR2 polypeptide (e.g., single-chain polypeptide, antibody, or antigen-binding fragment thereof) in the same cell proliferation assay in the absence of TNFα). Examples of cell death assays that can be used to measure the antagonistic effects of TNFR2 antibodies are described herein, e.g., in Example 2, below. Similarly, antagonistic TNFR2 polypeptides, such as single-chain polypeptides, antibodies, antigen-binding fragments thereof, and constructs, described herein may inhibit TNFR2 signaling as assessed by measuring the expression of one or more genes selected from the group consisting of CHUK, NFKBIE, NFKBIA, MAP3K11, TRAF2, TRAF3, relB, and clAP2/BIRC3 by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to such cells that are not treated with the TNFR2 antagonist polypeptide. The antagonistic TNFR2 polypeptide (e.g., single-chain polypeptide, antibody, or antigen-biding fragment thereof) may exhibit an IC₅₀ value in such a gene expression assay that is largely unchanged by the presence or absence of TNFα (e.g., an IC₅₀ value in the presence of TNFα that is changed by less than 50%, 45%, 40%, 35%, 25%, 20%, 15%, 10%, 9%, 8%, 7%, 6%, 5%, 4%, 3%, 2%, or less than 1% relative to the IC₅₀ value of the antagonistic TNFR2 polypeptide (e.g., single-chain polypeptide, antibody, or antigen-binding fragment thereof) in the same gene expression assay in the absence of TNFα).

### Direct Killing of T-Reg Cells, MDSCs, and TNFR2+ Cancer Cells

Antagonistic TNFR2 polypeptides disclosed herein, such as single-chain polypeptides, antibodies, antigen-binding fragments thereof, and constructs, may, for instance, not only reduce the proliferation of T-reg cells, TNFR2+ cancer cells, and/or MDSCs, but may also induce the death of T-reg cells, TNFR2+ cancer cells, and/or MDSCs within a sample (e.g., within a patient, such as a human patient). Antagonistic TNFR2 polypeptides described herein may be capable, for instance, of reducing the total quantity of T-reg cells, cancer cells (such as cutaneous T cell lymphoma cells, ovarian cancer cells, colon cancer cells, renal cell carcinoma cells or multiple myeloma cells, among others), and/or MDSCs in a sample treated with an antagonist TNFR2 antibody or antigen-binding fragment thereof (such as a sample isolated from a human patient undergoing treatment for cancer or an infectious disease as described herein) by, e.g., 1%, 2%, 3%, 4%, 5%, 6%, 7%, 8%, 9%, 10%, 1 5%, 20%, 25%, 30%, 35%, 40%, 45%, 50%, 55%, 60%, 65%, 70%, 75%, 80%, 85%, 90%, 95%, 100%, or more, relative to a sample not treated with an antagonist TNFR2 antibody or antigen-binding fragment thereof.

The ability of antagonistic TNFR2 polypeptides (e.g., single-chain polypeptides, antibodies, and antigen-binding fragments) described herein to attenuate T-reg, MDSC, and/or cancer cell growth may be due, in part, to the ability of these polypeptides to diminish the quantity of soluble TNFR2 within a sample (e.g., a sample isolated from a human patient undergoing treatment for cancer or an infectious disease as described herein). In the absence of this beneficial activity, soluble TNFR2 can be secreted by, e.g., T-reg cells, and could otherwise interfere with the ability of TNFR2 antagonists to localize to TNFR2 at the surface of a T-reg cell, TNFR2+ cancer cell, or MDSC by binding and sequestering such antagonists in the extracellular environment. By reducing TNFR2 secretion, antagonistic TNFR2 antibodies or antigen-binding fragments thereof described herein may render T-reg cells, TNFR2+ cancer cells, and/or MDSCs increasingly susceptible to therapeutic molecules, such as an antagonistic TNFR2 antibody or antigen-binding fragment thereof, and/or additional anti-cancer agents, such as those described herein or known in the art, that may be used in conjunction with the compositions and methods described herein.

Selective Modulation of Active (CD25^{Hi} and CD45RA^{Low}) T-Reg Cells

Antagonistic TNFR2 polypeptides (e.g., single-chain polypeptides, antibodies, antigen-binding fragments, and constructs) described herein may be capable of inhibiting the proliferation or reducing the total quantity of T-reg cells in a sample (e.g., a sample isolated from a human patient undergoing treatment for cancer or an infectious disease as described herein) and may act selectively on T-reg cells in an actively-dividing state. Antagonistic TNFR2 antibodies or antigen-binding fragments thereof described herein may selectively target active T-reg cells that express CD25^{Hi} and CD45RA^{Low}, e.g., over resting T-reg cells that express CD25^{Med} and CD45RA^{Hi}. For instance, antagonistic TNFR2 antibodies or antigen-binding fragments thereof described herein may be capable of reducing the proliferation of T-reg cells expressing CD25^{Hi} and CD45RA^{Low} by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more relative to T-reg cells that do not express the CD25^{Hi} and CD45RA^{Low} proteins, such as T-reg cells that express CD25^{Med} and CD45RA^{Hi} proteins.

### Modulation of T-Reg Cells, MDSCs, and T Effector Cells in the Tumor Microenvironment

Antagonist TNFR2 polypeptides described herein, such as single-chain polypeptides, antibodies, and antigen-binding fragments thereof, may reduce or inhibit the proliferation of T-reg cells with a greater potency in a patient suffering from cancer relative to a subject that does not have cancer. The antagonist TNFR2 polypeptides described herein, such as single-chain polypeptides, antibodies, and antigen-binding fragments thereof, may reduce or inhibit the proliferation of T-reg cells with a greater potency in the microenvironment of a tumor relative to a site that is free of cancer cells, such as a site distal from a tumor in a patient suffering from cancer or in a subject without cancer. This effect may be determined using, for example, a cell death assay as described herein. For instance, the polypeptides described herein, such as single-chain polypeptides, antibodies, antigen-binding fragments thereof, and constructs, may exhibit an IC₅₀ for reducing or inhibiting the proliferation of T-reg cells in the microenvironment of a tumor that is less than the IC₅₀ of the polypeptides for reducing or inhibiting the proliferation of T-reg cells in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. Examples of cell death assays that can be used to measure the antagonistic effects of anti-TNFR2 polypeptides are described herein, e.g., in Example 2, below. The polypeptides described herein, such as single-chain polypeptides, antibodies, antigen-binding fragments thereof, and constructs, may reduce or inhibit the proliferation of T-reg cells or may promote the apoptosis of T-reg cells with a potency that is greater in the microenvironment of a tumor containing TNFR2+ cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a patient suffering from one or more of the foregoing cancers or a in a subject without cancer.

Additionally, or alternatively, the polypeptides described herein, such as single-chain polypeptides, antibodies, antigen-binding fragments thereof, and constructs, may reduce or inhibit the proliferation of MDSCs with a greater potency in a patient suffering from cancer relative to a subject that does not have cancer. The polypeptides described herein, such as single-chain polypeptides, antibodies, antigen-binding fragments thereof, and constructs, may reduce or inhibit the proliferation of MDSCs with a greater potency in the microenvironment of a tumor relative to a site that is free of cancer cells, such as a site distal from a tumor in a patient suffering from cancer or in a subject without cancer. This effect may be determined using, for example, a cell death assay described herein. For instance, the polypeptides described herein, such as single-chain polypeptides, antibodies, antigen-binding fragments thereof, and constructs, may have an IC₅₀ for reducing or inhibiting the proliferation of MDSCs in the microenvironment of a tumor that is less than the IC₅₀ of the polypeptides for reducing or inhibiting the proliferation of MDSCs in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. Examples of cell death assays that can be used to measure the antagonistic effects of anti-TNFR2 polypeptides are described herein, e.g., in Example 2, below. The polypeptides described herein, such as single-chain polypeptides, antibodies, antigen-binding fragments thereof, and constructs, may reduce or inhibit the proliferation of MDSCs or may promote the apoptosis of MDSCs with a potency that is greater in the microenvironment of a tumor containing TNFR2+ cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a patient suffering from one or more of the foregoing cancers or a in a subject without cancer.

Additionally, or alternatively, the polypeptides described herein, such as single-chain polypeptides, antibodies, antigen-binding fragments thereof, and constructs, may expand T effector cells, such as CD8+ cytotoxic T cells, with a greater potency in a patient suffering from cancer relative to a subject that does not have cancer. In some embodiments, the polypeptides described herein, such as single-chain polypeptides, antibodies, and antigen-binding fragments thereof, expand T effector cells, such as CD8+ cytotoxic T cells, with a greater potency in the microenvironment of a tumor relative to a site that is free of cancer cells, such as a site distal from a tumor in a patient suffering from cancer or a in a subject without cancer. This effect may be determined using, for example, a cell proliferation assay described herein. For instance, the polypeptides described herein, such as single-chain polypeptides, antibodies, antigen-binding fragments thereof, and constructs, may have an EC₅₀ for the expansion of T effector cells in the microenvironment of a tumor that is less than the EC₅₀ of the polypeptides for expanding T effector cells in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. Examples of cell proliferation assays that can be used to measure the effects of anti-TNFR2 polypeptides on T effector cells are described herein, e.g., in Example 2, below. The polypeptides described herein, such as single-chain polypeptides, antibodies, antigen-binding fragments thereof, and constructs, may directly expand T effector cells, such as CD8+ cytotoxic T cells, with a potency that is greater in the microenvironment of a tumor containing TNFR2+ cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a patient suffering from one or more of the foregoing cancers or a in a subject without cancer. The T effector cells (e.g., CD8+ cytotoxic T cells) may, for example, specifically react with an antigen present on one or more cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, among cells of other cancers described herein.

### Activity of Antigen-Binding Fragments of Full-Length TNFR2 Antagonist Antibodies

Antagonistic TNFR2 antibodies described herein may inhibit, e.g., T-reg, cancer cell, and/or MDSC growth, or promote T effector cell growth, with a similar potency as that exhibited by antigen-binding fragments of such antibodies. For instance, removal of the Fc region of an antagonistic TNFR2 antibody described herein may not alter the ability of the molecule to attenuate the proliferation or reduce the total quantity of T-reg cells, MDSCs, and/or cancer cells in a sample (e.g., a sample isolated from a human patient undergoing treatment for cancer or an infectious disease as described herein). Antagonistic TNFR2 antibodies and antigen-binding fragments thereof described herein may function, for instance, by a pathway distinct from antibody-dependent cellular cytotoxicity (ADCC), in which an Fc region is required to recruit effector proteins in order to induce cell death. Additionally, antagonistic TNFR2 antibodies or antigen-binding fragments thereof may not be susceptible to a loss of inhibitory capacity in the presence of cross-linking agents. Antagonistic TNFR2 antibodies or antigen-binding fragments thereof described herein may therefore exhibit therapeutic activity in a variety of isotypes, such as IgG, IgA, IgM, IgD, or IgE, or in a variety of forms, such as a single-chain polypeptide (e.g., a single-chain polypeptide containing one or more CDRs covalently bound to one another, for instance, by an amide bond, a thioether bond, a carbon-carbon bond, or a disulfide bridge), a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

### Specific Binding Properties of Antagonistic TNFR2 Polypeptides

The specific binding of a polypeptide, such as a single-chain polypeptide, antibody, or antibody fragment described herein, to human TNFR2 can be determined by any of a variety of established methods. The affinity can be represented quantitatively by various measurements, including the concentration of antibody needed to achieve half-maximal inhibition of the TNFα-TNFR2 interaction in vitro (IC₅₀) and the equilibrium constant (K_{D}) of the antibody-TNFR2 complex dissociation. The equilibrium constant, K_{D}, that describes the interaction of TNFR2 with an antibody described herein is the chemical equilibrium constant for the dissociation reaction of a TNFR2-antibody complex into solvent-separated TNFR2 and antibody molecules that do not interact with one another.

Polypeptides (e.g., single-chain polypeptides, antibodies, and antigen-binding fragments) described herein include those that specifically bind to TNFR2 with a K_{D} value of less than 100 nM (e.g., 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, or 1 nM). In some embodiments, polypeptides (e.g., single-chain polypeptides, antibodies, antigen-binding fragments, and constructs) described herein specifically bind to TNFR2 with a K_{D} value of less than 1 nM (e.g., (e.g., 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, 10 pM, 5 pM, or 1 pM).

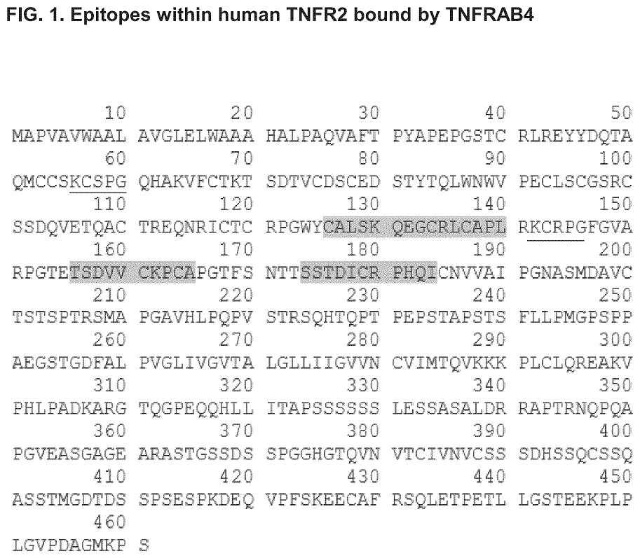

Polypeptides described herein can also be characterized by a variety of in vitro binding assays. Examples of experiments that can be used to determine the K_{D} or 1050 of an anti-TNFR2 polypeptide include, e.g., surface plasmon resonance, isothermal titration calorimetry, fluorescence anisotropy, and ELISA-based assays, among others. ELISA represents a particularly useful method for analyzing antibody activity, as such assays typically require minimal concentrations of antibodies. A common signal that is analyzed in a typical ELISA assay is luminescence, which is typically the result of the activity of a peroxidase conjugated to a secondary antibody that specifically binds a primary antibody (e.g., a TNFR2 antibody described herein). Polypeptides (e.g., single-chain polypeptides, antibodies, and antigen-binding fragments) described herein are capable of binding TNFR2 and epitopes therein, such as epitopes containing one or more continuous or discontinuous residues, or all residues, of (i) amino acids 174-184 of SEQ ID NO: 1 within human TNFR2 (SSTDICRPHQI (SEQ ID NO: 31), (ii) amino acids 126-140 of SEQ ID NO: 1 within human TNFR2 (CALSKQEGCRLCAPL (SEQ ID NO: 32), and/or (iii) amino acids 156-165 of SEQ ID NO: 1 within human TNFR2 (TSDVVCKPCA (SEQ ID NO: 33), as shown in FIG. 1. Antagonistic polypeptides described herein may additionally bind isolated peptides derived from TNFR2 that structurally pre-organize various residues in a manner that simulates the conformation of the above epitopes in the native protein. For instance, polypeptides (e.g., single-chain polypeptides, antibodies, antigen-binding fragments, and constructs) described herein may bind peptides containing the amino acid sequence of any one of SEQ ID NOs: 31-33, or a peptide having up to five amino acid substitutions with respect to the amino acid sequence of any one of SEQ ID NOs: 31-33 (such as a peptide having up to five conservative amino acid substitutions with respect to the amino acid sequence of any one of SEQ ID NOs: 31-33), and/or a peptide having an amino acid sequence that is at least 85% identical (e.g., 85%, 90%, 95%, 96%, 97%, 98%, 99%, or 100% identical) to the amino acid sequence of any one of SEQ ID NOs: 31-33. In a direct ELISA experiment, this binding can be quantified, e.g., by analyzing the luminescence that occurs upon incubation of an HRP substrate (e.g., 2,2′-azino-di-3-ethylbenzthiazoline sulfonate) with an antigen-antibody complex bound to a HRP-conjugated secondary antibody.

### Kinetic Properties of Antagonistic TNFR2 Polypeptides

In addition to the thermodynamic parameters of a TNFR2-polypeptide interaction, it is also possible to quantitatively characterize the kinetic association and dissociation of a polypeptide described herein with TNFR2. This can be done, e.g., by monitoring the rate of polypeptide-antigen (e.g., antibody-antigen) complex formation according to established procedures. For example, one can use surface plasmon resonance (SPR) to determine the rate constants for the formation (kₒₙ) and dissociation (kₒₙ) of an antibody-TNFR2 complex. These data also enable calculation of the equilibrium constant of (K_{D}) of antibody-TNFR2 complex dissociation, since the equilibrium constant of this unimolecular dissociation can be expressed as the ratio of the kₒₙ to kₒₙ values. SPR is a technique that is particularly advantageous for determining kinetic and thermodynamic parameters of receptor-antibody interactions since the experiment does not require that one component be modified by attachment of a chemical label. Rather, the receptor is typically immobilized on a solid metallic surface which is treated in pulses with solutions of increasing concentrations of antibody. Antibody-receptor binding induces distortion in the angle of reflection of incident light at the metallic surface, and this change in refractive index over time as antibody is introduced to the system can be fit to established regression models in order to calculate the association and dissociation rate constants of an antibody-receptor interaction.

Polypeptides (e.g., single-chain polypeptides, antibodies, antigen-binding fragments, and constructs) described herein may exhibit high kₒₙ and low kₒₙ values upon interaction with TNFR2, consistent with high-affinity receptor binding. For example, polypeptides described herein may exhibit kₒₙ values in the presence of TNFR2 of greater than 10⁴ M⁻¹s⁻¹ (e.g., 1.0×10⁴ M⁻¹s⁻¹, 1.5×10⁴ M⁻¹s⁻¹, 2.0×10⁴ M⁻¹s⁻¹, 2.5×10⁴ M⁻¹s⁻¹, 3.0×10⁴ M⁻¹s⁻¹, 3.5×10⁴ M⁻¹s⁻¹, 4.0×10⁴ M⁻¹s⁻¹, 4.5×10⁴ M⁻¹s⁻¹, 5.0×10⁴ M⁻¹s⁻¹, 5.5×10⁴ M⁻¹s⁻¹, 6.0×10⁴ M⁻¹s⁻¹, 6.5×10⁴ M⁻¹s⁻¹, 7.0×10⁴ M⁻¹s⁻¹, 7.5×10⁴ M⁻¹s⁻¹, 8.0×10⁴ M⁻¹s⁻¹, 8.5×10⁴ M⁻¹s⁻¹, 9.0×10⁴ M⁻¹s⁻¹, 9.5×10⁴ M⁻¹s⁻¹, 1.0×10⁵ M⁻¹s⁻¹, 1.5×10⁵ M⁻¹s⁻¹, 2.0×10⁵ M⁻¹s⁻¹, 2.5×10⁵ M⁻¹s⁻¹, 3.0×10⁵ M⁻¹s⁻¹, 3.5×10⁵ M-s⁻¹, 4.0×10⁵ M⁻¹s⁻¹, 4.5×10⁵ M⁻¹s⁻¹, 5.0×10⁵ M⁻¹s⁻¹, 5.5×10⁵ M⁻¹s⁻¹, 6.0×10⁵ M⁻¹s⁻¹, 6.5×10⁵ M⁻¹s⁻¹, 7.0×10⁵ M⁻¹s⁻¹, 7.5×10⁵ M⁻¹s⁻¹, 8.0×10⁵ M⁻¹s⁻¹, 8.5×10⁵ M⁻¹s⁻¹, 9.0×10⁵ M⁻¹s⁻¹, 9.5×10⁵ M⁻¹s⁻¹, or 1.0×10⁶ M⁻¹s⁻¹). Polypeptides (e.g., single-chain polypeptides, antibodies, antigen-binding fragments, and constructs) described herein may exhibit low k_{off} values when bound to TNFR2, as these polypeptides are capable of interacting with distinct TNFR2 epitopes with a high affinity. Residues within these epitopes may form strong intermolecular contacts with TFNR2, which can slow the dissociation of the antibody-TNFR2 complex. This high receptor affinity can manifest in low k_{off} values. For instance, polypeptides described herein may exhibit kₒₙ values of less than 10⁻³ s⁻¹ when complexed to TNFR2 (e.g., 1.0×10⁻³ s⁻¹, 9.5×10⁻⁴ s⁻¹, 9.0×10⁻⁴ s⁻¹, 8.5 ×10⁻⁴ s⁻¹, 8.0×10⁻⁴ s⁻¹, 7.5×10⁻⁴ s⁻¹, 7.0×10⁻⁴ s⁻¹, 6.5×10⁻⁴ s⁻¹, 6.0×10⁻⁴ s⁻¹, 5.5×10⁻⁴ s⁻¹, 5.0×10⁻⁴ s⁻¹, 4.5 ×10-4 s⁻¹, 4.0×10⁻⁴ s⁻¹, 3.5×10⁻⁴ s⁻¹, 3.0×10⁻⁴ s⁻¹, 2.5×10⁻⁴ s⁻¹, 2.0×10⁻⁴ s⁻¹, 1.5×10⁻⁴ s⁻¹, 1.0×10⁻⁴ s⁻¹, 9.5 ×10-5 s⁻¹, 9.0×10⁻⁵ s⁻¹, 8.5×10⁻⁵ s⁻¹, 8.0×10⁻⁵ s⁻¹, 7.5×10⁻⁵ s⁻¹, 7.0×10⁻⁵ s⁻¹, 6.5×10⁻⁵ s⁻¹, 6.0×10⁻⁵ s⁻¹, 5.5×10-5 s⁻¹, 5.0×10⁻⁵ s⁻¹, 4.5×10⁻⁵ s⁻¹, 4.0×10⁻⁵ s⁻¹, 3.5×10⁻⁵ s⁻¹, 3.0×10⁻⁵ s⁻¹, 2.5×10⁻⁵ s⁻¹, 2.0×10⁻⁵ s⁻¹, 1.5 ×10-5 s⁻¹, or 1.0×10⁻⁵ s⁻¹).

Epitopes within TNFR2 Bound by Antagonistic TNFR2 Polypeptides

Among the difficulties in developing anti-TNFR2 polypeptides (e.g., single-chain polypeptides, antibodies, and antigen-binding fragments) that are capable of antagonizing TNFR2 has been the elucidation of epitopes within TNFR2 that participate in antagonistic complex formation rather than epitopes that promote signal transduction. The present invention is based in part on the discovery of epitopes within TNFR2 that, when bound, promote receptor antagonism and the ability to promote one or more, or all, of the following advantageous biological activities:
(a) Suppression of the proliferation of, and/or the direct killing of, T-reg cells, for instance, by binding and inactivating TNFR2 on the T-reg cell surface;
(b) Suppression of the proliferation of, and/or the direct killing of, MDSCs, for instance, by binding and inactivating TNFR2 on the MDSC surface;
(c) Promotion of the expansion of T effector cells, such as CD8+ T cells; and/or
(d) Suppression of the proliferation of, and/or the direct killing of, TNFR2-expressing cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells.

Antagonistic TNFR2 polypeptides, such dominant antagonistic TNFR2 polypeptides (e.g., single-chain polypeptides, antibodies, antigen-binding fragments, and constructs) described herein specifically bind epitopes containing one or more, or all, of residues 174-184 of SEQ ID NO: 1 within human TNFR2 (SSTDICRPHQI, SEQ ID NO: 31), and/or one or more, or all, residues within an epitope that exhibits at least 85% sequence identity (e.g., 85%, 90%, 95%, 97%, 99%, or 100% sequence identity) to SEQ ID NO: 31, and/or one or more, or all, residues of an epitope that contains one or more (e.g., up to five) conservative amino acid substitutions relative to SEQ ID NO: 31. For instance, antagonistic TNFR2 polypeptides (e.g., single-chain polypeptides, antibodies, antigen-binding fragments, and constructs) described herein may specifically bind one or more continuous or discontinuous epitopes within residues 174-184 of SEQ ID NO: 1 within human TNFR2, and/or one or more continuous or discontinuous epitopes within a peptide that exhibits at least 85% sequence identity (e.g., 85%, 90%, 95%, 97%, 99%, or 100% sequence identity) to SEQ ID NO: 31, and/or one or more continuous or discontinuous epitopes within a peptide that contains one or more (e.g., up to five) conservative amino acid substitutions relative to SEQ ID NO: 31.

Additionally, or alternatively, antagonistic TNFR2 polypeptides, such dominant antagonistic TNFR2 polypeptides (e.g., single-chain polypeptides, antibodies, antigen-binding fragments, and constructs) described herein specifically bind epitopes containing one or more, or all, of residues 126-140 of SEQ ID NO: 1 within human TNFR2 (CALSKQEGCRLCAPL), SEQ ID NO: 32), and/or one or more, or all, residues within an epitope that exhibits at least 85% sequence identity (e.g., 85%, 90%, 95%, 97%, 99%, or 100% sequence identity) to SEQ ID NO: 32, and/or one or more, or all, residues of an epitope that contains one or more (e.g., up to five) conservative amino acid substitutions relative to SEQ ID NO: 32. For instance, antagonistic TNFR2 polypeptides (e.g., single-chain polypeptides, antibodies, antigen-binding fragments, and constructs) described herein may specifically bind one or more continuous or discontinuous epitopes within residues 126-140 of SEQ ID NO: 1 within human TNFR2, and/or one or more continuous or discontinuous epitopes within a peptide that exhibits at least 85% sequence identity (e.g., 85%, 90%, 95%, 97%, 99%, or 100% sequence identity) to SEQ ID NO: 32, and/or one or more continuous or discontinuous epitopes within a peptide that contains one or more (e.g., up to five) conservative amino acid substitutions relative to SEQ ID NO: 32.

Additionally, or alternatively, antagonistic TNFR2 polypeptides, such dominant antagonistic TNFR2 polypeptides (e.g., single-chain polypeptides, antibodies, antigen-binding fragments, and constructs) described herein specifically bind epitopes containing one or more, or all, of residues 156-165 of SEQ ID NO: 1 within human TNFR2 (TSDVVCKPCA), SEQ ID NO: 33), and/or one or more, or all, residues within an epitope that exhibits at least 85% sequence identity (e.g., 85%, 90%, 95%, 97%, 99%, or 100% sequence identity) to SEQ ID NO: 33, and/or one or more, or all, residues of an epitope that contains one or more (e.g., up to five) conservative amino acid substitutions relative to SEQ ID NO: 33. For instance, antagonistic TNFR2 polypeptides (e.g., single-chain polypeptides, antibodies, antigen-binding fragments, and constructs) described herein may specifically bind one or more continuous or discontinuous epitopes within residues 156-165 of SEQ ID NO: 1 within human TNFR2, and/or one or more continuous or discontinuous epitopes within a peptide that exhibits at least 85% sequence identity (e.g., 85%, 90%, 95%, 97%, 99%, or 100% sequence identity) to SEQ ID NO: 33, and/or one or more continuous or discontinuous epitopes within a peptide that contains one or more (e.g., up to five) conservative amino acid substitutions relative to SEQ ID NO: 33.

Antagonistic TNFR2 polypeptides described herein may specifically bind discontinuous epitopes within TNFR2 that contain two or more of the above epitopes. For instance, antagonistic TNFR2 polypeptides described herein may specifically bind one or more, or all, of the following discontinuous epitopes:
(a) a discontinuous epitope containing one or more, or all, of residues 174-184 of SEQ ID NO: 1 within human TNFR2 (SSTDICRPHQI, SEQ ID NO: 31), and one or more, or all, of residues 126-140 of SEQ ID NO: 1 within human TNFR2 (CALSKQEGCRLCAPL), SEQ ID NO: 32;
(b) a discontinuous epitope containing one or more, or all, of residues 174-184 of SEQ ID NO: 1 within human TNFR2 (SSTDICRPHQI, SEQ ID NO: 31), and one or more, or all, of residues 156-165 of SEQ ID NO: 1 within human TNFR2 (TSDVVCKPCA), SEQ ID NO: 33; and/or
(c) a discontinuous epitope containing one or more, or all, of residues 126-140 of SEQ ID NO: 1 within human TNFR2 (CALSKQEGCRLCAPL, SEQ ID NO: 32), and one or more, or all, of residues 156-165 of SEQ ID NO: 1 within human TNFR2 (TSDVVCKPCA), SEQ ID NO: 33.

In some embodiments, antagonistic TNFR2 polypeptides, such as dominant antagonistic TNFR2 polypeptides (e.g., single-chain polypeptides, antibodies, antigen-binding fragments, and constructs) described herein do not bind or more, or all, residues of 142-146 of SEQ ID NO: 1 within human TNFR2 (KCRPG, SEQ ID NO: 9). Additionally, antagonistic TNFR2 polypeptides described herein distinctly do not exhibit specific binding to an epitope containing residues 56-60 of SEQ ID NO: 1 within human TNFR2 (KCSPG, SEQ ID NO: 5). Polypeptides that exhibit the ability to bind one or more of the above epitopes within human TNFR2 and an epitope containing residues 56-60 of SEQ ID NO: 1 within human TNFR2 lack inhibitory (antagonistic) activity. As such, the ability of a TNFR2 polypeptide to discriminate among these epitopes and specifically interact with one or more of the epitopes described above and to not engage in specific binding with an epitope composed of residues 56-60 of SEQ ID NO: 1 within human TNFR2 characterizes polypeptides described herein that antagonize TNFR2 signaling.

One exemplary procedure that can be used to predict the inhibitory activity of a TNFR2 polypeptide described herein is to compare the affinity of the antibody or antibody fragment for a peptide containing the SSTDICRPHQI motif (SEQ ID NO: 31), the CALSKQEGCRLCAPL motif (SEQ ID NO: 32), or the TSDVVCKPCA motif (SEQ ID NO: 33), such as a linear or cyclic peptide that contains one or more of these motifs. The peptide may be, for example, structurally pre-organized by virtue of one or more conformational constraints (e.g., backbone or side-chain-to-side-chain cyclization) in a manner that simulates the three-dimensional orientation of one of more of the SSTDICRPHQI (SEQ ID NO: 31), CALSKQEGCRLCAPL (SEQ ID NO: 32), and/or TSDVVCKPCA (SEQ ID NO: 33) epitopes within native human TNFR2. For instance, antagonistic TNFR2 polypeptides described herein may specifically bind such a peptide with an affinity that is greater than that of the antagonistic TNFR2 polypeptide for a peptide fragment defined by residues 48-67 of SEQ ID NO: 1 within human TNFR2 (QTAQMCCSKCSPGQHAKVFC, SEQ ID NO: 8). For example, antagonistic TNFR2 polypeptides described herein may bind a peptide containing one of more of the SSTDICRPHQI (SEQ ID NO: 31), CALSKQEGCRLCAPL (SEQ ID NO: 32), or TSDVVCKPCA (SEQ ID NO: 33) epitopes within native human TNFR2 with an affinity that is, e.g., 10-fold, 15-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more than 1000-fold greater than the affinity of the antagonistic polypeptide for a peptide having the amino acid sequence of SEQ ID NO: 18.

Antagonistic TNFR2 Polypeptides that Bind TNFR2 from Non-Human Animals

In addition to binding epitopes within human TFNR2 that contain one or more, or all, residues of the SSTDICRPHQI (SEQ ID NO: 31), CALSKQEGCRLCAPL (SEQ ID NO: 32), or TSDVVCKPCA (SEQ ID NO: 33) motifs, antagonistic TNFR2 polypeptides, such as dominant antagonistic TNFR2 polypeptides (e.g., single-chain polypeptides, antibodies, antigen-binding fragments thereof, and constructs) described herein also include those that specifically bind epitopes containing one or more of the equivalent motifs within TNFR2 derived from non-human animals. The locations of epitopes equivalent to the human SSTDICRPHQI (SEQ ID NO: 31), CALSKQEGCRLCAPL (SEQ ID NO: 32), and TSDVVCKPCA (SEQ ID NO: 33) motifs in TNFR2 derived from exemplary non-human mammals is shown in Tables 2-4, below.

TABLE 2
Locations of sequences equivalent to SSTDICRPHQI in TNFR2 from non-human mammals

| | | | SEQ ID NO. of | Genbank |
|---|---|---|---|---|
| | Sequence | Amino acid positions of | full-length | Accession No. |
| Source of | equivalent to | equivalent sequence | TNFR2 | of full-length |
| TNFR2 | SSTDICRPHQI | within TNFR2 | sequence | TNFR2 sequence |
| Human | SSTDICRPHQI | 174-184 | 1 | P20333.3 |
| Cattle | SYTDTCKPHRN | 174-184 | 34 | AAI05223 |
| Bison | SYTDTCKPHRN | 174-184 | 28 | XP_010848145 |
| Mouse | SSTDVCRPHRI | 176-186 | 29 | AAA39752.1 |
| Rat | SSTDVCRPHRI | 176-186 | 30 | Q80WY6 |

TABLE 3
Locations of sequences equivalent to CALSKQEGCRLCAPL in TNFR2 from non-human mammals

| | | Amino acid | | |
|---|---|---|---|---|
| | | positions of | SEQ ID NO. of | |
| | | equivalent | full-length | Genbank |
| Source | Sequence equivalent to | sequence | TNFR2 | Accession No. |
| of TNFR2 | CALSKQEGCRLCAPL | within TNFR2 | sequence | of full-length |
| Human | CALSKQEG-CRLCAPL | 126-140 | 1 | P20333.3 |
| Cattle | CTLGRQEG-CRLCVAL | 126-140 | 34 | AAI05223 |
| Bison | CTLGRQEG-CRLCVAL | 126-140 | 28 | XP_010848145 |
| Mouse | CALKTHSGSCRQCMRL | 127-142 | 29 | AAA39752.1 |
| Rat | CALKLHSGNCRQCMKL | 127-142 | 30 | Q80WY6 |

TABLE 4
Locations of sequences equivalent to TSDVVCKPCA in TNFR2 from non-human mammals

| | | Amino acid | SEQ ID NO. | Genbank |
|---|---|---|---|---|
| | Sequence | positions of | of full-length | Accession No. |
| Source of | equivalent to | equivalent sequence | TNFR2 | of full-length |
| TNFR2 | TSDVVCKPCA | within TNFR2 | sequence | TNFR2 sequence |
| Human | TSDVVCKPCA | 156-165 | 1 | P20333.3 |
| Cattle | TTNVICAPCG | 156-165 | 34 | AAI05223 |
| Bison | TTNVICAPCG | 156-165 | 28 | XP 010848145 |
| Mouse | NGNVLCKACA | 158-167 | 29 | AAA39752.1 |
| Rat | NGNVICSACA | 158-167 | 30 | Q80WY6 |

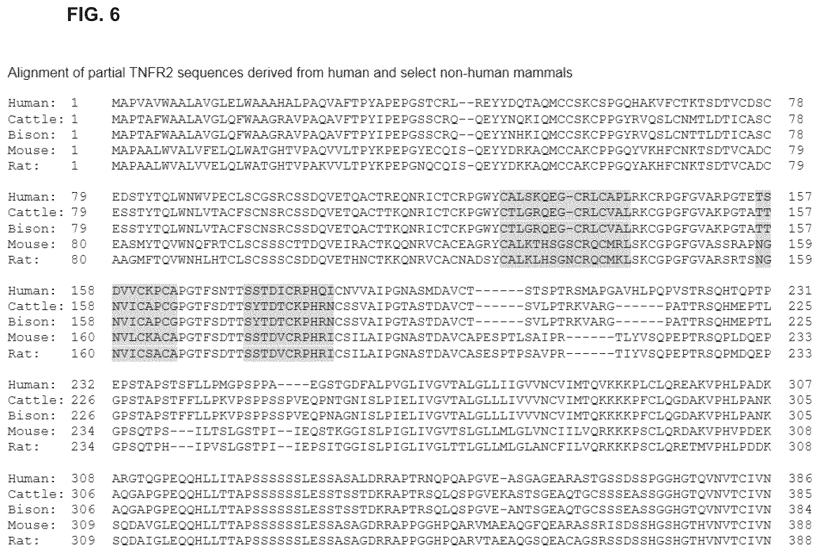

Epitopes within TNFR2 derived from the non-human mammals discussed above that may be bound by antagonistic TNFR2 polypeptides, such as dominant antagonistic TNFR2 polypeptides (e.g., single-chain polypeptides, antibodies, antigen-binding fragments, and constructs) described herein are illustrated in FIG. 6. This figure shows a partial sequence alignment of TNFR2 derived from human, cattle, bison, mouse, and rat, as well as epitopes (highlighted in grey) equivalent to the foregoing human motifs.

### The Antagonistic TNFR2 Antibody TNFRAB4

Antagonistic TNFR2 polypeptides, such as single-chain polypeptides, antibodies, antigen-binding fragments thereof, and constructs, described herein may exhibit binding properties that are the same as or similar to those of TNFRAB4. TNFRAB4 is a monoclonal murine antibody described herein. This antibody is a dominant TNFR2 antagonist, and exhibits the following beneficial properties:
(a) Suppression of the proliferation of, and/or the direct killing of, T-reg cells, for instance, by binding and inactivating TNFR2 on the T-reg cell surface;
(b) Suppression of the proliferation of, and/or the direct killing of, MDSCs, for instance, by binding and inactivating TNFR2 on the MDSC surface;
(c) Promotion of the expansion of T effector cells, such as CD8+ T cells; and/or
(d) Suppression of the proliferation of, and/or the direct killing of, TNFR2-expressing cancer cells, such as Hodgkin's lymphoma cells, cutaneous non-Hodgkin's lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells.

Monoclonal antibody TNFRAB4 binds epitopes within human TNFR2 containing the following amino acid residues:
(a) residues 174-184 of SEQ ID NO: 1 within human TNFR2 (SSTDICRPHQI, SEQ ID NO: 31);
(b) residues 126-140 of SEQ ID NO: 1 within human TNFR2 (CALSKQEGCRLCAPL), SEQ ID NO: 32); and
(c) residues 156-165 of SEQ ID NO: 1 within human TNFR2 (TSDVVCKPCA), SEQ ID NO: 33).

As described in detail below, antagonistic TNFR2 polypeptides described herein (e.g., single-chain polypeptides, antibodies, antigen-binding fragments thereof, and constructs) can be generated by producing and identifying antibodies that exhibit epitope-binding properties similar to those of TNFRAB4. Exemplary techniques for the production of polypeptides (e.g., single-chain polypeptides, antibodies, antigen-binding fragments thereof, and constructs) that have epitope-binding properties similar to those of TNFRAB4 include, without limitation, the production of fully human, humanized, primatized, and chimeric antibodies that incorporate one or more, or all, of the complementarity-determining regions (CDRs) of TNFRAB4, as well as screening for polypeptides that specifically bind one or more, or all, epitopes on TNFR2 that are specifically bound by TNFRAB4.

### Fully Human, Humanized, Primatized, and Chimeric Antibodies

Antibodies described herein include fully human, humanized, primatized, and chimeric antibodies that contain one or more, or all, of the CDR sequences of TNFRAB4 (e.g., the CDR-H1, CDR-H2, CDR-H3, CDR-L1, CDR-L2, and/or CDR-L3 sequences of TNFRAB4). Additionally, antibodies described herein include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4). Antagonistic TNFR2 antibodies described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of TNFRAB4. For example, antagonistic TNFR2 antibodies described herein can be generated by incorporating any one or more of the CDR sequences of TNFRAB4 into the framework regions (e.g., FW1, FW2, FW3, and FW4) of a human antibody. Exemplary framework regions that can be used for the development of a humanized anti-TNFR2 antibody containing one or more of the CDRs of TNFRAB4 include, without limitation, those described in U.S. Pat. Nos. 7,732,578, 8,093,068, and WO 2003/105782; the disclosures of each of which are incorporated herein by reference.

As an example, one strategy that can be used to design humanized antibodies described herein is to align the sequences of the heavy chain variable region and light chain variable region of TNFRAB4 with the heavy chain variable region and light chain variable region of a consensus human antibody. Consensus human antibody heavy chain and light chain sequences are known in the art (see e.g., the “VBASE” human germline sequence database; see also Kabat, et al., Sequences of Proteins of Immunological Interest, Fifth Edition, U.S. Department of Health and Human Services, NIH Publication No. 91-3242, 1991; Tomlinson et al., J. Mol. Biol. 227:776-98, 1992; and Cox et al, Eur. J. Immunol. 24:827-836, 1994; the disclosure of which is incorporated herein by reference). In this way, the variable domain framework residues and CDRs can be identified by sequence alignment (see Kabat, supra). One can substitute, for example, one or more of the CDRs of the consensus human antibody with the corresponding CDR(s) of TNFRAB4, in order to produce a humanized TNFR2 antagonist antibody. Exemplary variable domains of a consensus human antibody include the heavy chain variable domain:

(SEQ ID NO: 10)
EVQLVESGGGLVQPGGSLRLSCAASGFTFSDYAMSWVRQAPGKGLEWVAV
ISENGSDTYYADSVKGRFTISRDDSKNTLYLQMNSLRAEDTAVYYCARDR
GGAVSYFDVWGQGTLVTVSS

and the light chain variable domain:

(SEQ ID NO: 11)
DIQMTQSPSSLSASVGDRVTITCRASQDVSSYLAWYQQKPGKAPKLLIYA
ASSLESGVPSRFSGSGSGTDFTLTISSLQPEDFATYYCQQYNSLPYTFGQ
GTKVEIKRT

identified in U.S. Pat. No. 6,054,297; the disclosure of which is incorporated herein by reference (CDRs are shown in bold were determined according to the method of Chothia, et al., J. Mol. Biol, 196:901-917, 1987). These amino acid substitutions can be made, for example, by recombinant expression of polynucleotides encoding the heavy and light chains of a humanized antibody in a host cell using methods known in the art or described herein.

Similarly, this strategy can also be used to produce primatized antagonistic TNFR2 antibodies, as one can substitute, for example, one or more, or all, of the CDRs of a primate antibody consensus sequence with, for example, one or more, or all, of the CDRs of TNFRAB4. Consensus primate antibody sequences known in the art (see e.g., U.S. Pat. Nos. 5,658,570; 5,681,722; and 5,693,780; the disclosures of each of which are incorporated herein by reference).

In some embodiments, it may be desirable to import particular framework residues in addition to CDR sequences from an antagonistic TNFR2 antibody, such as TNFRAB4, into the heavy and/or light chain variable domains of a human antibody. For instance, U.S. Pat. No. 6,054,297 identifies several instances when it may be advantageous to retain certain framework residues from a particular antibody heavy chain or light chain variable region in the resulting humanized antibody. In some embodiments, framework residues may engage in non-covalent interactions with the antigen and thus contribute to the affinity of the antibody for the target antigen. In some embodiments, individual framework residues may modulate the conformation of a CDR, and thus indirectly influence the interaction of the antibody with the antigen. Certain framework residues may form the interface between V_{H} and V_{L} domains, and may therefore contribute to the global antibody structure. In some cases, framework residues may constitute functional glycosylation sites (e.g., Asn-X-Ser/Thr) which may dictate antibody structure and antigen affinity upon attachment to carbohydrate moieties. In cases such as those described above, it may be beneficial to retain certain framework residues of a TNFR2 antagonist antibody (e.g., TNFRAB4) in, e.g., a humanized or primatized antagonistic antibody or antigen-binding fragment thereof, as various framework residues may promote high epitope affinity and improved biochemical activity of the antibody or antigen-binding fragment thereof.

Antibodies described herein also include antibody fragments, Fab domains, F(ab′) molecules, F(ab′)₂ molecules, single-chain variable fragments (scFvs), tandem scFv fragments, diabodies, triabodies, dual variable domain immunoglobulins, multi-specific antibodies, bispecific antibodies, and heterospecific antibodies that contain one or more, or all, of the CDRs of TNFRAB4, or one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4). Antagonistic TNFR2 antibodies described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of TNFRAB4. These molecules can be expressed recombinantly, e.g., by incorporating polynucleotides encoding these proteins into expression vectors for transfection in a eukaryotic or prokaryotic cell using techniques described herein or known in the art, or synthesized chemically, e.g., by solid phase peptide synthesis methods described herein or known in the art.

Polypeptides described herein additionally include antibody-like scaffolds that contain, for example, one or more, or all, of the CDRs of TNFRAB4, or one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4) or contains one or more (for instance, up to 3) amino acid substitutions (e.g., one or more conservative amino acid substitutions) relative to the corresponding CDR sequence of TNFRAB4. Examples of antibody-like scaffolds include proteins that contain a tenth fibronectin type III domain (¹⁰Fn3), which contains BC, DE, and FG structural loops analogous to canonical antibodies. The tertiary structure of the ¹⁰Fn3 domain resembles that of the variable region of the IgG heavy chain, and one of skill in the art can graft, e.g., one or more, or all, of the CDR sequences of TNFRAB4 or sequences having at least 85% sequence identity (e.g., 90%, 95%, 97%, 99%, or 100% sequence identity) to any one or more of these CDR sequences or sequences containing amino acid substitutions, such as conservative or nonconservative amino acid substitutions (e.g., up to 3 amino acid substitutions) relative to one or more of these CDR sequences onto the fibronectin scaffold by replacing residues of the BC, DE, and FG loops of ¹⁰Fn3 with residues of the corresponding CDR sequence of TNFRAB4. This can be achieved by recombinant expression of a modified ¹⁰Fn3 domain in a prokaryotic or eukaryotic cell (e.g., using the vectors and techniques described herein). Examples of using the ¹⁰Fn3 domain as an antibody-like scaffold for the grafting of CDRs from antibodies onto the BC, DE, and FG structural loops are reported in WO 2000/034784, WO 2009/142773, WO 2012/088006, and U.S. Pat. No. 8,278,419; the disclosures of each of which are incorporated herein by reference.

### Molecular Determinants of TNFR2 Affinity and Antagonism

The antagonistic TNFR2 polypeptides, such as dominant antagonistic TNFR2 polypeptides, of the disclosure (e.g., a single-chain polypeptide, antibody, antigen-binding fragment thereof, or construct thereof described herein) may contain a complementarity-determining region (CDR) heavy chain 1 (CDR1) having the amino acid sequence GJTF(J)₂Y (SEQ ID NO: 50) or GJTF(J)₂YJ (SEQ ID NO: 51), in which each J is independently a naturally occurring amino acid. In some embodiments, the polypeptide (e.g., a single-chain polypeptide, antibody, antigen-binding fragment thereof, or construct thereof) further contains:
(a) a CDR-H2 having the amino acid sequence (J)₃GSJ or (J)₅GSJ;
(b) a CDR-H3 having the amino acid sequence JRJDGJSJY(J)₂FDJ (SEQ ID NO: 35) or JRJDGSY(J)₂FD(J)₃ (SEQ ID NO: 36);
(c) a CDR-L1 having the amino acid sequence (J)₉Y or (J)₅Y;
(d) a CDR-L2 having the amino acid sequence (J)₆S or (J)₂S; and/or
(e) a CDR-L3 having the amino acid sequence (J)₅Y(J)₂T or (J)₃Y(J)₄T, in which each J is independently a naturally occurring amino acid.

The polypeptide (e.g., a single-chain polypeptide, antibody, antigen-binding fragment thereof, or construct thereof) may contain a CDR-H1 having the amino acid sequence Z⁴FZ³Z⁵SSZ⁵ or Z⁴YZ³Z⁵TDZ⁵X; In which each Z³ is independently an amino acid including a polar, uncharged side-chain at physiological pH;
each Z⁴ is independently a glycine or alanine;
each Z⁵ is independently an amino acid including a hydrophobic side-chain; and
each X is independently leucine or isoleucine.

In some embodiments, the polypeptide (e.g., a single-chain polypeptide, antibody, antigen-binding fragment thereof, or construct thereof) further contains:
(a) a CDR-H2 having the amino acid sequence SSGZ⁴Z³Y (SEQ ID NO: 52) or VDPEYZ⁴Z³T (SEQ ID NO: 43);
(b) a CDR-H3 having the amino acid sequence QZ¹VZ²Z⁴YZ³SZ⁵WYZ⁵Z²Z⁵ (SEQ ID NO: 37) or AZ¹DZ²Z⁴Z³Z⁵SPZ⁵Z²Z⁵WG (SEQ ID NO: 38);
(c) a CDR-L1 having the amino acid sequence SASSSVYYMZ⁵ (SEQ ID NO: 53) or QNINKZ⁵ (SEQ ID NO: 44);
(d) a CDR-L2 having the amino acid sequence STSNLAZ³ (SEQ ID NO: 54), TYZ³, or YTZ³; and/or
(e) a CDR-L3 having the amino acid sequence QQRRNZ⁵PYZ³ (SEQ ID NO: 55) or CLQZ⁵VNLXZ³ (SEQ ID NO: 45);
in which each Z¹ is independently an amino acid including a cationic side-chain at physiological pH;
each Z² is independently an amino acid including an anionic side-chain at physiological pH;
each Z³ is independently an amino acid including a polar, uncharged side-chain at physiological pH;
each Z⁴ is independently a glycine or alanine;
each Z⁵ is independently an amino acid including a hydrophobic side-chain; and
each X is independently leucine or isoleucine.

The antagonistic TNFR2 polypeptides (e.g., a dominant antagonistic TNFR2 polyleptide) of the disclosure (e.g., a single-chain polypeptide, antibody, antigen-binding fragment thereof, or construct thereof) may contain a CDR-H1 having the amino acid sequence GFTFSSY (SEQ ID NO: 56), GYTFTDYX (SEQ ID NO: 57), or an amino acid sequence having up to two amino acid substitutions (e.g., conservative amino acid substitutions) relative to these sequences, in which each X is independently leucine or isoleucine, optionally in which the amino acid substitutions are conservative amino acid substitutions. In some embodiments, the polypeptide (e.g., a single-chain polypeptide, antibody, antigen-binding fragment thereof, or construct thereof) further contains:
(a) a CDR-H2 having the amino acid sequence SSGGSY (SEQ ID NO: 58), VDPEYGST (SEQ ID NO: 47), or an amino acid sequence having up to two amino acid substitutions (e.g., conservative amino acid substitutions) relative to these sequences;
(b) a CDR-H3 having the amino acid sequence QRVDGYSSYWYFDV (SEQ ID NO: 39), ARDDGSYSPFDYWG (SEQ ID NO: 40), or an amino acid sequence having up to two amino acid substitutions (e.g., conservative amino acid substitutions) relative to these sequences;
(c) a CDR-L1 having the amino acid sequence SASSSVYYMY (SEQ ID NO: 59), QNINKY (SEQ ID NO: 48), or an amino acid sequence having up to two amino acid substitutions (e.g., conservative amino acid substitutions) relative to these sequences;
(d) a CDR-L2 having the amino acid sequence STSNLAS (SEQ ID NO: 60), TYS, YTS, or an amino acid sequence having up to two amino acid substitutions (e.g., conservative amino acid substitutions) relative to SEQ ID NO: 60; and/or
(e) a CDR-L3 having the amino acid sequence QQRRNYPYT (SEQ ID NO: 61), CLQYVNLXT (SEQ ID NO: 49), or an amino acid sequence having up to two amino acid substitutions (e.g., conservative amino acid substitutions) relative to these sequences.

In some embodiments, the polypeptide (e.g., a single-chain polypeptide, antibody, antigen-binding fragment thereof, or construct thereof) contains a heavy chain including one or more of the following CDRs:
(a) a CDR-H1 having the amino acid sequence GFTFSSY (SEQ ID NO: 56);
(b) a CDR-H2 having the amino acid sequence SSGGSY (SEQ ID NO: 58); and
(c) a CDR-H3 having the amino acid sequence QRVDGYSSYWYFDV (SEQ ID NO: 39).

The polypeptide (e.g., a single-chain polypeptide, antibody, antigen-binding fragment thereof, or construct thereof) may contain, for example, a heavy chain having one or more of the following CDRs:
(a) a CDR-H1 having the amino acid sequence GYTFTDYX (SEQ ID NO: 57);
(b) a CDR-H2 having the amino acid sequence VDPEYGST (SEQ ID NO: 47); and
(c) a CDR-H3 having the amino acid sequence ARDDGSYSPFDYWG (SEQ ID NO: 40);
in which each X is independently leucine or isoleucine.

In some embodiments, the CDR-H1 has the amino acid sequence GYTFTDYL (SEQ ID NO: 46). In some embodiments, the CDR-H1 has the amino acid sequence GYTFTDYI (SEQ ID NO: 62). In some embodiments, the CDR-H1 has the amino acid sequence GYTFTDVI (SEQ ID NO: 63). In some embodiments, the CDR-H1 has the amino acid sequence GYTFTDYS (SEQ ID NO: 64).

Additionally or alternatively, the polypeptide (e.g., a single-chain polypeptide, antibody, antigen-binding fragment thereof, or construct thereof) may contain, for example, a light chain having one or more of the following CDRs:
(a) a CDR-L1 having the amino acid sequence SASSSVYYMY (SEQ ID NO: 59);
(b) a CDR-L2 having the amino acid sequence STSNLAS (SEQ ID NO: 60); and
(c) a CDR-L3 having the amino acid sequence QQRRNYPYT (SEQ ID NO: 61).

In some embodiments, the antibody or antigen-binding fragment thereof contains a light chain having one or more of the following CDRs:
(a) a CDR-L1 having the amino acid sequence QNINKY (SEQ ID NO: 48);
(b) a CDR-L2 having the amino acid sequence TYS or YTS; and
(c) a CDR-L3 having the amino acid sequence CLQYVNLXT (SEQ ID NO: 49);
in which each X is independently leucine or isoleucine.

In some embodiments, the CDR-L2 has the amino acid sequence TYS. In some embodiments, the CDR-L2 has the amino acid sequence YTS. The CDR-L3 may have the amino acid sequence CLQYVNLLT (SEQ ID NO: 65). In some embodiments, the CDR-L3 has the amino acid sequence CLQYVNLIT (SEQ ID NO: 66).

The polypeptide (e.g., a single-chain polypeptide, antibody, antigen-binding fragment thereof, or construct thereof) may contain three heavy chain CDRs, including:
(a) a CDR-H1 having the amino acid sequence GFTFSSY (SEQ ID NO: 56);
(b) a CDR-H2 having the amino acid sequence SSGGSY (SEQ ID NO: 58); and
(c) a CDR-H3 having the amino acid sequence QRVDGYSSYWYFDV (SEQ ID NO: 39);
and may further contain three light chain CDRs, including:
(d) a CDR-L1 having the amino acid sequence SASSSVYYMY (SEQ ID NO: 59);
(e) a CDR-L2 having the amino acid sequence STSNLAS (SEQ ID NO: 60); and
(f) a CDR-L3 having the amino acid sequence QQRRNYPYT (SEQ ID NO: 61).

In some embodiments, polypeptide (e.g., single-chain polypeptides, antibody, antigen-binding fragment thereof, or construct thereof) contains three heavy chain CDRs, including:
(a) a CDR-H1 having the amino acid sequence GYTFTDYX (SEQ ID NO: 57), such as GYTFTDYL (SEQ ID NO: 46) or GYTFTDYI (SEQ ID NO: 62);
(b) a CDR-H2 having the amino acid sequence VDPEYGST (SEQ ID NO: 47); and
(c) a CDR-H3 having the amino acid sequence ARDDGSYSPFDYWG (SEQ ID NO: 40);
and further contains three light chain CDRs, including:
(d) a CDR-L1 having the amino acid sequence QNINKY (SEQ ID NO: 48);
(e) a CDR-L2 having the amino acid sequence TYS or YTS; and
(f) a CDR-L3 having the amino acid sequence CLQYVNLXT (SEQ ID NO: 49), such as CLQYVNLLT (SEQ ID NO: 65) or CLQYVNLIT (SEQ ID NO: 66);
in which each X is independently leucine or isoleucine.

In some embodiments, the polypeptide (e.g., a single-chain polypeptide, antibody, antigen-binding fragment thereof, or construct thereof) includes a framework region having the amino acid sequence LLIR (SEQ ID NO: 67) bound to the N-terminus of the CDR-L2 and/or a framework region having the amino acid sequence TLE bound to the C-terminus of the CDR-L2.

The antagonistic TNFR2 polypeptides (e.g., a dominant antagonistic TNFR2 polyleptide) of the disclosure (e.g., a single-chain polypeptide, antibody, antigen-binding fragment thereof, or construct thereof) may have a heavy chain variable domain having an amino acid sequence that is at least 85% identical (e.g., at least 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 68. In some embodiments, the heavy chain variable domain has an amino acid sequence that is at least 90% identical (e.g., at least 90%, 91%, 92%, 93%, 94%, 95%, 96%, 97%, 98%, 99%, 99.9%, or 100% identical) to the amino acid sequence of SEQ ID NO: 68. In some embodiments, the heavy chain variable domain has an amino acid sequence that is at least 95% identical (e.g., at least 95%, 96%, 97%, 98%, 99%, 99.9%, or 100% identical) to the amino acid sequence of SEQ ID NO: 68.

Additionally or alternatively, the polypeptide (e.g., a single-chain polypeptide, antibody, antigen-binding fragment thereof, or construct thereof) may have a light chain variable domain having an amino acid sequence that is at least 85% identical (e.g., at least 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 69. In some embodiments, the light chain variable domain has an amino acid sequence that is at least 90% identical (e.g., at least 90%, 91%, 92%, 93%, 94%, 95%, 96%, 97%, 98%, 99%, 99.9%, or 100% identical) to the amino acid sequence of SEQ ID NO: 69. In some embodiments, the light chain variable domain has an amino acid sequence that is at least 95% identical (e.g., at least 95%, 96%, 97%, 98%, 99%, 99.9%, or 100% identical) to the amino acid sequence of SEQ ID NO: 69.

Antagonistic TNFR2 polypeptides, such dominant antagonistic TNFR2 polypeptides (e.g., single-chain polypeptides, antibodies, or antigen-binding fragments thereof), described herein may contain a CDR-H3 represented by the formula JZ¹JZ²Z⁴JZ³JZ⁵(J)₂Z⁵Z²Z⁵ or JZ¹JZ²Z⁴Z³Z⁵(J)₂Z⁵Z²Z⁵(J)₂, wherein each J is independently a naturally occurring amino acid, each Z¹ is independently a naturally occurring amino acid containing a cationic side-chain at physiological pH, each Z² is independently a naturally occurring amino acid containing an anionic side-chain at physiological pH, each Z³ is independently a naturally occurring amino acid containing a polar, uncharged side-chain at physiological pH, each Z⁴ is independently a glycine or alanine, and each Z⁵ is independently a naturally occurring amino acid containing a hydrophobic side-chain.

Similarly, the antagonistic TNFR2 polypeptides described herein, such as dominant antagonistic TNFR2 polypeptides (e.g., single-chain polypeptides, antibodies, or antigen-binding fragments thereof), may contain a CDR-H3 represented by the formula JRJDGJSJY(J)₂FDJ (SEQ ID NO: 35), JRJDGSY(J)₂FD(J)₃ (SEQ ID NO: 36), QZ¹VZ²Z⁴YZ³SZ⁵WYZ⁵Z²Z⁵ (SEQ ID NO: 37), or AZ¹DZ²Z⁴Z³Z⁵SPZ⁵Z²Z⁵WG (SEQ ID NO: 38). For instance, the CDR-H3 may have the amino acid sequence QRVDGYSSYWYFDV (SEQ ID NO: 39). The CDR-H3 may have the amino acid sequence ARDDGSYSPFDYWG (SEQ ID NO: 40). In some embodiments, the CDR-H3 has the amino acid sequence ARDDGSYSPFDYFG (SEQ ID NO: 41).

Additionally, or alternatively, the antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence Z⁴JZ³Z⁵(J)₂Z⁵J; a CDR-H2 having the amino acid sequence (J)₅Z⁴Z³J; a CDR-L1 having the amino acid sequence (J)₅Z⁵; a CDR-L2 having the amino acid sequence (J)₂Z³; and/or a CDR-L3 having the amino acid sequence (J)₃Z⁵(J)₄Z³; wherein each J is independently a naturally occurring amino acid; each Z¹ is independently a naturally occurring amino acid containing a cationic side-chain at physiological pH; each Z² is independently a naturally occurring amino acid containing an anionic side-chain at physiological pH; each Z³ is independently a naturally occurring amino acid containing a polar, uncharged side-chain at physiological pH; each Z⁴ is independently a glycine or alanine; and each Z⁵ is independently a naturally occurring amino acid containing a hydrophobic side-chain.

In some embodiments, the antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence GJTF(J)₂YL (SEQ ID NO: 42); a CDR-H2 having the amino acid sequence (J)₅GSJ; a CDR-L1 having the amino acid sequence (J)₅Y; a CDR-L2 having the amino acid sequence (J)₂S; and/or a CDR-L3 having the amino acid sequence (J)₃Y(J)₄T; wherein each J is independently a naturally occurring amino acid.

The antagonistic TNFR2 polypeptides described herein, such as dominant antagonistic TNFR2 polypeptides (e.g., single-chain polypeptides, antibodies, or antigen-binding fragments thereof) may have a CDR-H1 having the amino acid sequence Z⁴YZ³Z⁵TDZ⁵L; a CDR-H2 having the amino acid sequence VDPEYZ⁴Z³T (SEQ ID NO: 43); a CDR-L1 having the amino acid sequence QNINKZ⁵ (SEQ ID NO: 44); a CDR-L2 having the amino acid sequence TYZ³ or YTZ³; and/or a CDR-L3 having the amino acid sequence CLQZ⁵VNLXZ³ (SEQ ID NO: 45); wherein each Z¹ is independently an amino acid containing a cationic side-chain at physiological pH; each Z² is independently an amino acid containing an anionic side-chain at physiological pH; each Z³ is independently an amino acid containing a polar, uncharged side-chain at physiological pH; each Z⁴ is independently a glycine or alanine; each Z⁵ is independently an amino acid containing a hydrophobic side-chain; and each X is independently leucine or isoleucine.

In some embodiments, the antagonistic TNFR2 polypeptides described herein, such as dominant antagonistic TNFR2 polypeptides (e.g., single-chain polypeptides, antibodies, or antigen-binding fragments thereof), may have a CDR-H1 having the amino acid sequence GYTFTDYL (SEQ ID NO: 46), or an amino acid sequence having up to two amino acid substitutions relative to this sequence, provided that the CDR-H1 preserves the C-terminal leucine residue of SEQ ID NO: 46; a CDR-H2 having the amino acid sequence VDPEYGST (SEQ ID NO: 47), or an amino acid sequence having up to two amino acid substitutions relative to this sequence; a CDR-L1 having the amino acid sequence QNINKY (SEQ ID NO: 48), or an amino acid sequence having up to two amino acid substitutions relative to this sequence; a CDR-L2 having the amino acid sequence TYS or YTS; and/or a CDR-L3 having the amino acid sequence CLQYVNLXT (SEQ ID NO: 49), or an amino acid sequence having up to two amino acid substitutions relative to this sequence.

### Antagonistic TNFR2 Single-Chain Polypeptides

TNFR2 antagonists described herein may be in the form of a single-chain polypeptide, such as a single-chain polypeptide that contains one or more, or all, of the CDRs of TNFRAB4, or one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4) or contains one or more (for instance, up to 3) amino acid substitutions (e.g., one or more conservative amino acid substitutions) relative to the corresponding CDR sequence of TNFRAB4. Single-chain polypeptides may be in the form of an antibody fragment, e.g., an antibody fragment described herein or known in the art, such as a scFv fragment. Single chain polypeptides may alternatively contain one or more CDRs described herein covalently bound to one another using conventional bond-forming techniques known in the art, for instance, by an amide bond, a thioether bond, a carbon-carbon bond, or by a linker, such as a peptide linker or a linker formed by nucleophilic substitution of a multi-valent electrophile (e.g., a bis(bromomethyl) arene derivative, such as a bis(bromomethyl)benzene or bis(bromomethyl)pyridine) described herein or known in the art.

Single-chain polypeptides can be produced by a variety of recombinant and synthetic techniques, such as by recombinant gene expression or solid-phase peptide synthesis procedures described herein or known in the art. For instance, one of skill in the art can design polynucleotides encoding, e.g., two or more CDRs operably linked to one another in frame so as to produce a continuous, single-chain peptide containing these CDRs. Optionally, the CDRs may be separated by a spacer, such as by a framework region (e.g., a framework sequence described herein or a framework region of a germline consensus sequence of a human antibody) or a flexible linker, such as a poly-glycine or glycine/serine linker described herein or known in the art. When produced by chemical synthesis methods, native chemical ligation can optionally be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference. A detailed description of techniques for the production of single-chain polypeptides, full-length antibodies, and antibody fragments is provided in the sections that follow.

### Nucleic Acids and Expression Systems

Antagonistic TNFR2 polypeptides (e.g., single-chain polypeptides, antibodies, antigen-binding fragments thereof, and constructs) described herein can be prepared by any of a variety of established techniques. For instance, an antagonistic TNFR2 antibody or antigen-binding fragment thereof described herein can be prepared by recombinant expression of immunoglobulin light and heavy chain genes in a host cell. To express an antibody recombinantly, a host cell can be transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel et al., eds., Greene Publishing Associates, 1989), and in U.S. Pat. No. 4,816,397; the disclosures of each of which are incorporated herein by reference.

### Vectors for Expression of Antagonistic TNFR2 Polypeptides

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); the disclosures of each of which are incorporated herein by reference.

### Genome Editing Techniques

In addition to viral vectors, a variety of additional methods have been developed for the incorporation of genes, e.g., those encoding antibody light and heavy chains, single-chain polypeptides, single-chain variable fragments (scFvs), tandem scFvs, Fab domains, F(ab′)₂ domains, diabodies, and triabodies, among others, into the genomes of target cells for polypeptide expression. One such method that can be used for incorporating polynucleotides encoding anti-TNFR2 polypeptides (e.g., single-chain polypeptides, antibodies, antigen-binding fragments thereof, or constructs) into prokaryotic or eukaryotic cells includes transposons. Transposons are polynucleotides that encode transposase enzymes and contain a polynucleotide sequence or gene of interest flanked by excision sites at the 5′ and 3′ positions. Once a transposon has been delivered into a cell, expression of the transposase gene commences and results in active enzymes that cleave the gene of interest from the transposon. This activity is mediated by the site-specific recognition of transposon excision sites by the transposase. In some embodiments, these excision sites may be terminal repeats or inverted terminal repeats. Once excised from the transposon, the gene of interest can be integrated into the genome of a prokaryotic or eukaryotic cell by transposase-catalyzed cleavage of similar excision sites that exist within nuclear genome of the cell. This allows the gene encoding an anti-TNFR2 antibody or fragment or domain thereof to be inserted into the cleaved nuclear DNA at the excision sites, and subsequent ligation of the phosphodiester bonds that join the gene of interest to the DNA of the prokaryotic or eukaryotic cell genome completes the incorporation process. In some embodiments, the transposon may be a retrotransposon, such that the gene encoding the antibody is first transcribed to an RNA product and then reverse-transcribed to DNA before incorporation in the prokaryotic or eukaryotic cell genome. Exemplary transposon systems include the piggybac transposon (described in detail in WO 2010/085699) and the sleeping beauty transposon (described in detail in US20050112764); the disclosures of each of which are incorporated herein by reference.

Another useful method for the integration of nucleic acid molecules encoding anti-TNFR2 polypeptides (e.g., single-chain polypeptides, antibodies, or antigen-binding fragments thereof) into the genome of a prokaryotic or eukaryotic cell is the clustered regularly interspaced short palindromic repeats (CRISPR)/Cas system, which is a system that originally evolved as an adaptive defense mechanism in bacteria and archaea against infection by viruses. The CRISPR/Cas system consists of palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al., Nat. Biotech., 31:227-229, 2013) and can be used as an efficient means of site-specifically editing eukaryotic or prokaryotic genomes in order to cleave DNA prior to the incorporation of a polynucleotide encoding an anti-TNFR2 polypeptides (e.g., single-chain polypeptides, antibodies, or antigen-binding fragments thereof) described herein. The use of CRISPR/Cas to modulate gene expression has been described in U.S. Pat. No. 8,697,359, the disclosure of which is incorporated herein by reference.

Alternative methods for site-specifically cleaving genomic DNA prior to the incorporation of a polynucleotide encoding a TNFR2 antibody or antibody fragment described herein include the use of zinc finger nucleases and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. Zinc finger nucleases and TALENs for use in genome editing applications are described in Urnov et al. (Nat. Rev. Genet., 11:636-646, 2010); and in Joung et al., (Nat. Rev. Mol. Cell. Bio. 14:49-55, 2013); incorporated herein by reference. Additional genome editing techniques that can be used to incorporate polynucleotides encoding antibodies described herein into the genome of a prokaryotic or eukaryotic cell include the use of ARCUS™ meganucleases that can be rationally designed so as to site-specifically cleave genomic DNA. The use of these enzymes for the incorporation of polynucleotides encoding antagonistic TNFR2 polypeptides (e.g., single-chain polypeptides, antibodies, antigen-binding fragments thereof, or constructs) described herein into the genome of a prokaryotic or eukaryotic cell is particularly advantageous in view of the structure-activity relationships that have been established for such enzymes. Single-chain meganucleases can thus be modified at certain amino acid positions in order to create nucleases that selectively cleave DNA at desired locations. These single-chain nucleases have been described extensively, e.g., in U.S. Pat. Nos. 8,021,867 and 8,445,251; the disclosures of each of which are incorporated herein by reference.

### Polynucleotide Sequence Elements

To express antagonistic TNFR2 polypeptides (e.g., single-chain polypeptides, antibodies, antigen-binding fragments thereof, or constructs) described herein, polynucleotides encoding partial or full-length light and heavy chains, e.g., polynucleotides that encode a one or more, or all, of the CDR sequences of TNFRAB4 or similar CDRs described as herein, can be inserted into expression vectors such that the genes are operatively linked to transcriptional and translational control sequences. The expression vector and expression control sequences are chosen to be compatible with the expression host cell used. Polynucleotides encoding the light chain gene and the heavy chain of a TNFR2 antibody can be inserted into separate vectors, or, optionally, both polynucleotides can be incorporated into the same expression vector using established techniques described herein or known in the art.

In addition to polynucleotides encoding the heavy and light chains of an antibody (or a polynucleotide encoding a single-chain polypeptide, an antibody fragment, such as a scFv molecule, or a construct described herein), the recombinant expression vectors described herein may carry regulatory sequences that control the expression of the antibody chain genes in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Viral regulatory elements, and sequences thereof, are described in detail, for instance, in U.S. Pat. Nos. 5,168,062, 4,510,245, and 4,968,615, the disclosures of each of which are incorporated herein by reference.

In addition to the antibody chain genes and regulatory sequences, the recombinant expression vectors described herein can carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR” host cells with methotrexate selection/amplification) and the neo gene (for G418 selection). In order to express the light and heavy chains of a TNFR2 antibody or a TNFR2 antibody fragment, the expression vector(s) containing polynucleotides encoding the heavy and light chains can be transfected into a host cell by standard techniques.

### Polynucleotides Encoding Modified Antagonistic TNFR2 Polypeptides

Antagonistic TNFR2 polypeptides (e.g., single-chain polypeptides, antibodies, antigen-binding fragments thereof, or constructs) described herein may contain one or more, or all, of the CDRs of TNFRAB4, or one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4) or contains one or more (for instance, up to 3) amino acid substitutions (e.g., one or more conservative amino acid substitutions) relative to the corresponding CDR sequence of TNFRAB4, but may feature differences in one or more framework regions of TNFRAB4. For instance, one or more framework regions of TNFRAB4 may be substituted with the framework region of a human antibody. Exemplary framework regions include, for example, human framework regions described in U.S. Pat. No. 7,829,086, and primate framework regions as described in EP 1945668; the disclosures of each of which are incorporated herein by reference. To generate nucleic acids encoding such TNFR2 antibodies, DNA fragments encoding, e.g., at least one, or both, of the light chain variable regions and the heavy chain variable regions can be produced by chemical synthesis (e.g., by solid phase polynucleotide synthesis techniques), in vitro gene amplification (e.g., by polymerase chain reaction techniques), or by replication of the polynucleotide in a host organism. For instance, nucleic acids encoding antagonistic TNFR2 antibodies described herein may be obtained by amplification and modification of germline DNA or cDNA encoding light and heavy chain variable sequences so as to incorporate one or more, or all, of the CDRs of TNFRAB4 into the framework residues of a consensus antibody.

In some embodiments, a humanized antagonistic TNFR2 antibody may include one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4) or contains one or more (for instance, up to 3) amino acid substitutions (e.g., one or more conservative amino acid substitutions) relative to the corresponding CDR sequence of TNFRAB4. This can be achieved, for example, by performing site-directed mutagenesis of germline DNA or cDNA and amplifying the resulting polynucleotides using the polymerase chain reaction (PCR) according to established procedures. Germline DNA sequences for human heavy and light chain variable region genes are known in the art (see, e.g., the “VBASE” human germline sequence database; see also Kabat et al., Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al., Eur. J. Immunol. 24:827-836, 1994; incorporated herein by reference). Chimeric nucleic acid constructs encoding human heavy and light chain variable regions containing one or more, or all, of the CDRs of TNFRAB4, or a similar sequence as described above, can be produced, e.g., using established cloning techniques known in the art. Additionally, a polynucleotide encoding a heavy chain variable region containing the one or more of the CDRs of TNFRAB4, or a similar sequence as described above, can be synthesized and used as a template for mutagenesis to generate a variant as described herein using routine mutagenesis techniques. Alternatively, a DNA fragment encoding the variant can be directly synthesized (e.g., by established solid phase nucleic acid chemical synthesis procedures).

Once DNA fragments encoding V_{H} segments containing one or more, or all, of the CDR-H1, CDR-H2, and CDR-H3 sequences of TNFRAB4 are obtained, these DNA fragments can be further manipulated by standard recombinant DNA techniques, e.g., to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker.

The isolated DNA encoding the V_{H} region of an antagonistic TNFR2 antibody described herein can be converted to a full-length heavy chain gene (as well as a Fab heavy chain gene), e.g., by operatively linking the V_{H}-encoding DNA to another DNA molecule encoding heavy chain constant region domains (C_{H}1, C_{H}2, C_{H}3, and, optionally, C_{H}4). The sequences of human heavy chain constant region genes are known in the art (see e.g., Kabat et al., Sequences of Proteins of Immunological Interest, Fifth Edition, U.S. Department of Health and Human Services, NIH Publication No. 91-3242, 1991) and DNA fragments encompassing these regions can be obtained by standard PCR amplification. The heavy chain constant region can be an IgG1, IgG2, IgG3, IgG4, IgA, IgE, IgM or IgD constant region, and in certain embodiments is an IgG1 constant region. For a Fab fragment heavy chain gene, the V_{H}-encoding DNA can be operatively linked to another DNA molecule encoding only the heavy chain C_{H}1 domain.

Isolated DNA encoding the V_{L} region of an antagonistic TNFR2 antibody can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat et al., Sequences of Proteins of Immunological Interest, Fifth Edition (U.S. Department of Health and Human Services, NIH Publication No. 91-3242, 1991)) and DNA fragments encompassing these regions can be obtained, e.g., by amplification in a prokaryotic or eukaryotic cell of a polynucleotide encoding these regions, by PCR amplification, or by chemical polynucleotide synthesis. The light chain constant region can be a kappa (κ) or lambda (A) constant region, but in certain embodiments is a kappa constant region. To create a scFv gene, the V_{H} and V_{L}-encoding DNA fragments are operatively linked to another fragment encoding a flexible linker, e.g., a polynucleotide encoding a flexible, hydrophilic amino acid sequence, such as the amino acid sequence (Gly₄Ser)₃, such that the V_{H} and V_{L} sequences can be expressed as a contiguous single-chain protein, with the V_{L} and V_{H} regions joined by the linker (see e.g., Bird et al., Science 242:423-426, 1988; Huston et al., Proc. Natl. Acad. Sci. USA 85:5879-5883, 1988; McCafferty et al., Nature 348:552-554, 1990).

Recombinant DNA technology can also be used to remove some or all of the DNA encoding either or both of the light and heavy chains that is not necessary for binding to TNFR2. The molecules expressed from such truncated DNA molecules are also encompassed by the antibodies described herein. In addition, bifunctional antibodies can be produced in which one heavy contains one or more, or all, of the CDRs of TNFRAB4, or a similar CDR sequence as described above, and the other heavy chain and/or the light chains are specific for an antigen other than TNFR2. Such antibodies can be generated, e.g., by crosslinking a heavy chain and light chain containing one or more, or all, of the CDRs of TNFRAB4, or a similar CDR sequence as described above, to a heavy chain and light chain of a second antibody specific for a different antigen, for instance, using standard chemical crosslinking methods (e.g., by disulfide bond formation). Bifunctional antibodies can also be made by expressing a nucleic acid molecule engineered to encode a bifunctional antibody in a prokaryotic or eukaryotic cell.

Dual specific antibodies, i.e., antibodies that bind TNFR2 and a different antigen using the same binding site, can be produced by mutating amino acid residues in the light chain and/or heavy chain CDRs. In some embodiments, dual specific antibodies that bind two antigens, such as TNFR2 and a second cell-surface receptor, can be produced by mutating amino acid residues in the periphery of the antigen binding site (Bostrom et al., Science 323: 1610-1614, 2009). Dual functional antibodies can be made by expressing a polynucleotide engineered to encode a dual specific antibody.

Modified antagonistic TNFR2 antibodies and antibody fragments described herein can also be produced by chemical synthesis (e.g., by the methods described in Solid Phase Peptide Synthesis, 2nd ed., 1984 The Pierce Chemical Co., Rockford, 111; incorporated herein by reference). Variant antibodies can also be generated using a cell-free synthetic platform (see, e.g., Chu et al., Biochemia No. 2, 2001 (Roche Molecular Biologicals); incorporated herein by reference).

### Host Cells for Expression of Antagonistic TNFR2 Polypeptides

It is possible to express the polypeptides (e.g., single-chain polypeptides, antibodies, antigen-binding fragments thereof, and constructs) described herein in either prokaryotic or eukaryotic host cells. In certain embodiments, expression of polypeptides (e.g., single-chain polypeptides, antibodies, or antigen-binding fragments thereof) is performed in eukaryotic cells, e.g., mammalian host cells, for optimal secretion of a properly folded and immunologically active antibody. Exemplary mammalian host cells for expressing the recombinant antibodies or antigen-binding fragments thereof described herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO myeloma cells, COS cells, 293 cells, and SP2/0 cells. Additional cell types that may be useful for the expression of antibodies and fragments thereof include bacterial cells, such as BL-21(DE3) E. colicells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Polypeptides (e.g., single-chain polypeptides, antibodies, antigen-binding fragments thereof, and constructs) can be recovered from the culture medium using standard protein purification methods. Host cells can also be used to produce portions of intact antibodies, such as Fab fragments or scFv molecules. Also included herein are methods in which the above procedure is varied according to established protocols known in the art. For example, it can be desirable to transfect a host cell with DNA encoding either the light chain or the heavy chain (but not both) of an antagonistic TNFR2 antibody described herein in order to produce an antigen-binding fragment of the antibody.

Once an antagonistic TNFR2 polypeptide (e.g., single-chain polypeptide, antibody, antigen-binding fragment thereof, or construct) described herein has been produced by recombinant expression, it can be purified by any method known in the art, such as a method useful for purification of an immunoglobulin molecule, for example, by chromatography (e.g., ion exchange, affinity, particularly by affinity for TNFR2 after Protein A or Protein G selection, and sizing column chromatography), centrifugation, differential solubility, or by any other standard technique for the purification of proteins. Further, the antagonistic TNFR2 polypeptides described herein or fragments thereof can be fused to heterologous polypeptide sequences described herein or otherwise known in the art to facilitate purification or to produce therapeutic conjugates (see “Antagonistic TNFR2 polypeptide conjugates,” below).

Once isolated, an anti-TNFR2 single-chain polypeptide, antibody, or antigen-binding fragments thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### Platforms for Generating and Affinity-Maturing Antagonistic Anti-TNFR2 Polypeptides

Mapping Epitopes of TNFR2 that Promote Receptor Antagonism

Antagonistic TNFR2 polypeptides (e.g., single-chain polypeptides, antibodies, antigen-binding fragments thereof, and constructs) described herein can be produced by screening libraries of polypeptides (e.g., single-chain polypeptides, antibodies, antigen-binding fragments thereof, and constructs) for functional molecules that are capable of binding epitopes within TNFR2 that selectively promote receptor antagonism rather than receptor activation. Such epitopes can be modeled by screening antibodies or antigen-binding fragments thereof against a series of linear or cyclic peptides containing residues that correspond to a desired epitope within TNFR2.

As an example, peptides containing individual fragments isolated from TNFR2 that promote receptor antagonism can be synthesized by peptide synthesis techniques described herein or known in the art. These peptides can be immobilized on a solid surface and screened for molecules that bind antagonistic TNFR2 polypeptides (e.g., single-chain polypeptides, antibodies, antigen-binding fragments thereof, and constructs), such as TNFRAB4, e.g., using an ELISA-based screening platform using established procedures. Using this assay, peptides that specifically bind TNFRAB4, with high affinity therefore contain residues within epitopes of TNFR2 that preferentially bind these antibodies. Peptides identified in this manner (e.g., peptides having the sequence of any one of SEQ ID NOs: 31-33) can be used to screen libraries of polypeptides (e.g., single-chain polypeptides, antibodies, antigen-binding fragments thereof, and constructs) in order to identify antagonistic TNFR2 polypeptides. Moreover, since these peptides act as surrogates for epitopes within TNFR2 that promote receptor antagonism, polypeptides generated using this screening technique may bind the corresponding epitopes in TNFR2 and are expected to be antagonistic of receptor activity.

### Screening of Libraries for Antagonistic TNFR2 Polypeptides

Methods for high throughput screening of polypeptide (e.g., single-chain polypeptide, antibody, antibody fragment, or construct) libraries for molecules capable of binding epitopes within TNFR2 (e.g., peptides having the sequence of any one of SEQ ID NOs: 31-33) include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see EP 0349578A, EP 4527839A, EP 0589877A; Chiswell and McCafferty (Trends Biotechnol. 10, 80-84 1992)). In addition, functional antibody fragments (e.g. Fab, single-chain Fv [scFv]) have been expressed (McCafferty et al. (Nature 348: 552-554, 1990), Barbas et al. (Proc. Natl. Acad Sci. USA 88:7978-7982, 1991), Clackson et al. (Nature 352:624-628, 1991)). These references are hereby incorporated by reference in their entirety.

(i) Phage Display Techniques

As an example, phage display techniques can be used in order to screen libraries of polypeptides (e.g., single-chain polypeptides, antibodies, antigen-binding fragments thereof, and constructs) for functional molecules capable of binding cyclic or polycyclic peptides containing epitopes within TNFR2 that promote receptor antagonism (e.g., peptides having the sequence of any one of SEQ ID NOs: 31-33). For instance, libraries of polynucleotides encoding single-chain antibody fragments, such as scFv fragments, that contain randomized hypervariable regions can be obtained using established procedures (e.g., solid phase polynucleotide synthesis or error-prone PCR techniques, see McCullum et al. (Meth. Mol. Biol., 634:103-109, 2010); incorporated herein by reference). These randomized polynucleotides can subsequently be incorporated into a viral genome such that the randomized antibody chains encoded by these genes are expressed on the surface of filamentous phage, e.g., by a covalent bond between the antibody chain and a coat protein (e.g., pIII coat protein on the surface of M13 phage). This provides a physical connection between the genotype and phenotype of the antibody chain. In this way, libraries of phage that display diverse antibody chains containing random mutations in hypervariable regions can be screened for the ability of the exterior antibody chains to bind TNFR2 epitopes (e.g., peptides having the sequence of any one of SEQ ID NOs: 31-33) that are immobilized to a surface using established procedures. For instance, such peptides can be physically bound to the surface of a microtiter plate by forming a covalent bond between the peptide and an epitope tag (e.g., biotin) and incubating the peptide in wells of a microtiter plate that have been previously coated with a complementary tag (e.g., avidin) that binds the tag attached to the peptide with high affinity. Suitable epitope tags include, without limitation, maltose-binding protein, glutathione-S-transferase, a poly-histidine tag, a FLAG-tag, a myc-tag, human influenza hemagglutinin (HA) tag, biotin, streptavidin. Peptides containing the epitopes presented by these molecules are capable of being immobilized on surfaces containing such complementary molecules as maltose, glutathione, a nickel-containing complex, an anti-FLAG antibody, an anti-myc antibody, an anti-HA antibody, streptavidin, or biotin, respectively. In this way, phage can be incubated with a surface containing an immobilized TNFR2-derived peptide for a time suitable to allow binding of the antibody to the constrained peptide and in the presence of an appropriate buffer system (e.g., one that contains physiological salt concentration, ionic strength, and is maintained at physiological pH by a buffering agent). The surface can then be washed (e.g., with phosphate buffer containing 0.1% Tween-20) so as to remove phage that do not present antibody chains that interact with the TNFR2-derived peptides with an affinity greater than a particular threshold value.

The affinity of the polypeptides that remain after this initial panning (i.e., screening) step can be modulated by adjusting the conditions of the washing step (e.g., by including mildly acidic or basic components, or by including other TNFR2-derived peptides at a low concentration in order to compete with immobilized peptides for antigen-binding sites). In this way, the population of phage that remains bound to the surfaces of the microtiter plate following the washing step is enriched for phage that bind TNFR2-derived peptide epitopes that promote receptor antagonism. The remaining phage can then be amplified by eluting the phage from the surface containing these peptides (e.g., by altering the ambient pH, ionic strength, or temperature) so as to diminish protein-protein interaction strength. The isolated phage can then be amplified, e.g., by infecting bacterial cells, and the resulting phage can optionally be subjected to panning by additional iterations of screening so as to further enrich the population of phage for those harboring higher-affinity anti-TNFR2 polypeptides. Following these panning stages, phage that display high-affinity antibodies or antigen-binding fragments thereof can subsequently be isolated and the genomes of these phage can be sequenced in order to identify the polynucleotide and polypeptide sequences of the encoded antibodies. Phage display techniques such as this can be used to generate, e.g., antibody chains, such as scFv fragments, tandem scFv fragments, and other antigen-binding fragments described herein that can be used as antagonists of TNFR2. Exemplary phage display protocols for the identification of antibody chains and antigen-binding fragments thereof that bind a particular antigen with high affinity are well-established and are described, e.g., in U.S. Pat. No. 7,846,892, WO 1997/002342, U.S. Pat. No. 8,846,867, and WO 2007/132917; the disclosures of each of which are incorporated herein by reference. Similar phage display techniques can be used to generate antibody-like scaffolds (e.g., ¹⁰Fn3 domains) described herein that bind epitopes within TNFR2 that promote receptor antagonism (e.g., epitopes presented by peptides with the sequence of any one of SEQ ID NOs: 31-33). Exemplary phage display protocols for the identification of antibody-like scaffold proteins are described, e.g., in WO 2009/086116; the disclosure of which is incorporated herein by reference).

(ii) Cell-Based Display Techniques

Other in vitro display techniques that exploit the linkage between genotype and phenotype of a solvent-exposed polypeptide include yeast and bacterial display. Yeast display techniques are established in the art and are often advantageous in that high quantities of antibodies (often up to 30,000) can be presented on the surface of an individual yeast cell (see, e.g., Boder et al. (Nat Biotechno. 15:553, 1997); incorporated herein by reference). The larger size of yeast cells over filamentous phage enables an additional screening strategy, as one can use flow cytometry to both analyze and sort libraries of yeast. For instance, established procedures can be used to generate libraries of bacterial cells or yeast cells that express polypeptides (e.g., single-chain polypeptides, antibodies, and antigen-binding fragments thereof) containing randomized hypervariable regions (see, e.g., see U.S. Pat. No. 7,749,501 and US 2013/0085072; the teachings of each which are incorporated herein by reference). For instance, large libraries of yeast cells that express polynucleotides encoding naïve scFv fragments can be made using established procedures (de Bruin et al., Nat Biotechnol 17:397, 1999; incorporated herein by reference). Yeast cells expressing these polynucleotides can then be incubated with two different fluorescent molecules during the panning steps: one dye that binds conserved residues within the antibody and thus reflects the amount of antibody displayed, and another dye that fluoresces at a different wavelength and binds the antigen and thus indicates the amount of antigen bound. For instance, one of skill in the art can use a TNFR2-derived peptide containing the sequence of any one of SEQ ID NOs: 31-33 that has been conjugated to an epitope tag (e.g., biotin), optionally at the N- or C-terminus of the peptide or at a residue that is not expected to interfere with antibody-antigen binding. This enables a fluorescent dye labeled with a complementary tag (e.g., avidin) to localize to the antibody-antigen complex. This results in great flexibility and immediate feedback on the progress of a selection. In contrast to phage display, by normalizing to antibody display levels, antibodies with higher affinities, rather than greater expression levels can easily be selected. In fact, it is possible to distinguish and sort antibodies whose affinities differ by only two-fold (VanAntwerp and Wittrup (Biotechnol Prog 16:31, 2000)).

(iii) Nucleotide Display Techniques

Display techniques that utilize in vitro translation of randomized polynucleotide libraries also provide a powerful approach to generating antagonistic TNFR2 polypeptides described herein. For instance, randomized DNA libraries encoding polypeptides (e.g., single-chain polypeptides, antibodies, and antigen-binding fragments thereof) that contain mutations within designated hypervariable regions can be obtained, e.g., using established PCR-based mutagenesis techniques as described herein. The polynucleotides of these libraries may contain transcription regulating sequences, such as promoters and transcription terminating sequences, and may additionally encode sequences that increase the rate of translation of the resulting mRNA construct (e.g., IRES sequences, 5′ and 3′ UTRs, a poly-adenylation tract, etc.). These polynucleotide libraries can be incubated in an appropriately buffered solution containing RNA polymerase and RNA nucleoside triphosphates (NTPs) in order to enable transcription of the DNA sequences to competent mRNA molecules, which can subsequently be translated by large and small ribosomal subunits, aminoacyl tRNA molecules, and translation initiation and elongation factors present in solution (e.g., using the PURExpress® In Vitro Protein Synthesis Kit, New England Biolabs®). Designed mRNA modifications can enable the antibody product to remain covalently bound to the mRNA template by a chemical bond to puromycin (e.g., see Keefe (Curr. Protoc. Mol. Biol., Chapter 24, Unit 24.5, 2001); incorporated herein by reference). This genotype-phenotype linkage can thus be used to select for antibodies that bind a TNFR2-derived peptide (e.g., a peptide that has the sequence of any one of SEQ ID NOs: 31-33) by incubating mRNA:antibody fusion constructs with a peptide immobilized to a surface and panning in a fashion similar to phage display techniques (see, e.g., WO 2006/072773; incorporated herein by reference).

Optionally, polypeptides (e.g., single-chain polypeptides, antibodies, and antigen-binding fragments thereof) described herein can be generated using a similar technique, except the antibody product may be bound non-covalently to the ribosome-mRNA complex rather than covalently via a puromycin linker. This platform, known as ribosome display, has been described, e.g., in U.S. Pat. No. 7,074,557; incorporated herein by reference. Alternatively, antibodies can be generated using cDNA display, a technique analogous to mRNA display with the exception that cDNA, rather than mRNA, is covalently bound to an antibody product via a puromycin linker. cDNA display techniques offer the advantage of being able to perform panning steps under increasingly stringent conditions, e.g., under conditions in which the salt concentration, ionic strength, pH, and/or temperature of the environment is adjusted in order to screen for antibodies with particularly high affinity for TNFR2-derived peptides. This is due to the higher natural stability of double-stranded cDNA over single-stranded mRNA. cDNA display screening techniques are described, e.g., in Ueno et al. (Methods Mol. Biol., 805:113-135, 2012); incorporated herein by reference.

In addition to generating antagonistic TNFR2 polypeptides (e.g., single-chain polypeptides, antibodies, antigen-binding fragments thereof, and constructs) described herein, in vitro display techniques (e.g., those described herein and those known in the art) also provide methods for improving the affinity of an antagonistic TNFR2 polypeptide described herein. For instance, rather than screening libraries of antibodies and fragments thereof containing completely randomized hypervariable regions, one can screen narrower libraries of antibodies and antigen-binding fragments thereof that feature targeted mutations at specific sites within hypervariable regions. This can be accomplished, e.g., by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for antibodies or antigen-binding fragments thereof that specifically bind TNFR2 epitopes (e.g., peptides containing the sequence of any one of SEQ ID NOs: 31-33) with improved binding affinity. Yeast display, for instance, is well-suited for affinity maturation, and has been used previously to improve the affinity of a single-chain antibody to a K_{D} of 48 fM (Boder et al. (Proc Natl Acad Sci USA 97:10701, 2000)).

Additional in vitro techniques that can be used for the generation and affinity maturation of antagonistic TNFR2 polypeptides (e.g., single-chain polypeptides, antibodies, and antigen-binding fragments thereof) described herein include the screening of combinatorial libraries of antibodies or antigen-binding fragments thereof for functional molecules capable of specifically binding TNFR2-derived peptides (e.g., a peptide having the amino acid sequence of any one of SEQ ID NOs: 31-33). Combinatorial antibody libraries can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins.

Libraries of combinatorial libraries thus obtained can be screened, e.g., by incubating a heterogeneous mixture of these antibodies with a peptide derived from TNFR2 that has been immobilized to a surface (e.g., a peptide having the amino acid sequence of any one of SEQ ID NOs: 31-33 immobilized to the surface of a solid-phase resin or a well of a microtiter plate) for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20). Antibodies that remain bound can subsequently be detected, e.g., using an ELISA-based detection protocol (see, e.g., U.S. Pat. No. 4,661,445; the disclosure of which is incorporated herein by reference).

Additional techniques for screening combinatorial libraries of polypeptides (e.g., single-chain polypeptides, antibodies, antigen-binding fragments thereof, and constructs) for those that specifically bind TNFR2-derived peptides (e.g., a peptide containing the amino acid sequence of any one of SEQ ID NOs: 31-33) include the screening of one-bead-one-compound libraries of antibody fragments. Antibody fragments can be chemically synthesized on a solid bead (e.g., using established split-and-pool solid phase peptide synthesis protocols) composed of a hydrophilic, water-swellable material such that each bead displays a single antibody fragment. Heterogeneous bead mixtures can then be incubated with a TNFR2-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody fragments that specifically bind a TNFR2-derived peptide (e.g., a peptide containing the amino acid sequence of any one of SEQ ID NOs: 31-33) can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind TNFR2-derived peptides can thus be separated from those that do not contain high-affinity antibody fragments. The sequence of an antibody fragment that specifically binds a TNFR2-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; the disclosures of each of which are incorporated herein by reference).

### Negative Screens of Polypeptides

In addition to the above-described methods for screening for a single-chain polypeptide, antibody, or antibody fragment that specifically binds to an epitope derived from human TNFR2 that promotes receptor antagonism, one can additionally perform negative screens in order to eliminate antibodies or antibody fragments that may also bind an epitope that contains the KCSPG sequence. For instance, mixtures of antibodies or antibody fragments isolated as a result of any of the above-described screening techniques can be screened for antibodies or antibody fragments that also specifically bind to a peptide derived from human TNFR2 that contains the KCSPG motif, such as a peptide containing residues 48-67 of SEQ ID NO: 1 (QTAQMCCSKCSPGQHAKVFC, SEQ ID NO: 8). This can be accomplished using any of the above-described methods or variations thereof, e.g., such that the antibodies or antibody fragments being screened are those that were previously identified as being capable of specifically binding a peptide containing one or more residues of SEQ ID NOs: 31-33. Exemplary techniques useful for a negative screen include those described above or known in the art, such as phage display, yeast display, bacterial display, ribosome display, mRNA display, cDNA display, or surface-based combinatorial library screens (e.g., in an ELISA format). This screening technique represents a useful strategy for identifying an antagonistic TNFR2 antibody or antibody fragment, as antibodies or antibody fragments capable of binding TNFR2 epitopes containing the KCSPG sequence lack, or have significantly reduced, antagonistic activity.

### Immunization of a Non-Human Mammal

Another strategy that can be used to produce antagonistic TNFR2 antibodies and antigen-binding fragments thereof described herein includes immunizing a non-human mammal. Examples of non-human mammals that can be immunized in order to produce antagonistic TNFR2 antibodies and fragments thereof described herein include rabbits, mice, rats, goats, guinea pigs, hamsters, horses, and sheep, as well as non-human primates. For instance, established procedures for immunizing primates are known in the art (see, e.g., WO 1986/6004782; incorporated herein by reference). Immunization represents a robust method of producing monoclonal antibodies by exploiting the antigen specificity of B lymphocytes. For example, monoclonal antibodies can be prepared by the Kohler-Millstein procedure (described, e.g., in EP 0110716; incorporated herein by reference), wherein spleen cells from a non-human animal (e.g., a primate) immunized with a peptide that presents a TNFR2-derived antigen that promotes receptor antagonism (e.g., a peptide containing the amino acid sequence of any one of SEQ ID NOs: 31-33). A clonally-expanded B lymphocyte produced by immunization can be isolated from the serum of the animal and subsequently fused with a myeloma cell in order to form a hybridoma. Hybridomas are particularly useful agents for antibody production, as these immortalized cells can provide a lasting supply of an antigen-specific antibody. Antibodies from such hybridomas can subsequently be isolated using techniques known in the art, e.g., by purifying the antibodies from the cell culture medium by affinity chromatography, using reagents such as Protein A or Protein G.

### Antagonistic TNFR2 Polypeptide Conjugates

Prior to administration of antagonistic TNFR2 polypeptides (e.g., single-chain polypeptides, antibodies, and antigen-binding fragments thereof) described herein to a mammalian subject (e.g., a human), it may be desirable to conjugate the antibody or fragment thereof to a second molecule, e g., to modulate the activity of the antibody in vivo. Antagonistic TNFR2 antibodies and fragments thereof can be conjugated to other molecules at either the N-terminus or C-terminus of a light or heavy chain of the antibody using any one of a variety of established conjugation strategies that are well-known in the art. Examples of pairs of reactive functional groups that can be used to covalently tether an antagonistic TNFR2 antibody or fragment thereof to another molecule include, without limitation, thiol pairs, carboxylic acids and amino groups, ketones and amino groups, aldehydes and amino groups, thiols and alpha,beta-unsaturated moieties (such as maleimides or dehydroalanine), thiols and alpha-halo amides, carboxylic acids and hydrazides, aldehydes and hydrazides, and ketones and hydrazides.

Antagonistic TNFR2 polypeptides (e.g., single-chain polypeptides, antibodies, antigen-binding fragments thereof, and constructs) can be covalently appended directly to another molecule by chemical conjugation as described. Alternatively, fusion proteins containing antagonistic TNFR2 antibodies and fragments thereof can be expressed recombinantly from a cell (e.g., a eukaryotic cell or prokaryotic cell). This can be accomplished, for example, by incorporating a polynucleotide encoding the fusion protein into the nuclear genome of a cell (e.g., using techniques described herein or known in the art). Optionally, antibodies and fragments thereof described herein can be joined to a second molecule by forming a covalent bond between the antibody and a linker. This linker can then be subsequently conjugated to another molecule, or the linker can be conjugated to another molecule prior to ligation to the anti-TNFR2 antibody or fragment thereof. Examples of linkers that can be used for the formation of a conjugate include polypeptide linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

### Drug-Polypeptide Conjugates

An antagonistic TNFR2 polypeptide (e.g., single-chain polypeptide, antibody, and antigen-binding fragment thereof) described herein can additionally be conjugated to, admixed with, or administered separately from a therapeutic agent, such as a cytotoxic molecule. Conjugates described herein may be applicable to the treatment or prevention of a disease associated with aberrant cell proliferation, such as a cancer described herein. Exemplary cytotoxic agents that can be conjugated to, admixed with, or administered separately from an antagonistic TNFR2 polypeptide include, without limitation, antineoplastic agents such as: acivicin; aclarubicin; acodazole hydrochloride; acronine; adozelesin; adriamycin; aldesleukin; altretamine; ambomycin; a. metantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; camptothecin; caracemide; carbetimer; carboplatin; carmustine; carubicin hydrochloride; carzelesin; cedefingol; chlorambucil; cirolemycin; cisplatin; cladribine; combretestatin a-4; crisnatol mesylate; cyclophosphamide; cytarabine; dacarbazine; daca (n-[2-(dimethyl-amino) ethyl] acridine-4-carboxamide); dactinomycin; daunorubicin hydrochloride; daunomycin; decitabine; dexormaplatin; dezaguanine; dezaguanine mesylate; diaziquone; docetaxel; dolasatins; doxorubicin; doxorubicin hydrochloride; droloxifene; droloxifene citrate; dromostanolone propionate; duazomycin; edatrexate; eflornithine hydrochloride; ellipticine; elsamitrucin; enloplatin; enpromate; epipropidine; epirubicin hydrochloride; erbulozole; esorubicin hydrochloride; estramustine; estramustine phosphate sodium; etanidazole; ethiodized oil i 131; etoposide; etoposide phosphate; etoprine; fadrozole hydrochloride; fazarabine; fenretinide; floxuridine; fludarabine phosphate; fluorouracil; 5-fdump; flurocitabine; fosquidone; fostriecin sodium; gemcitabine; gemcitabine hydrochloride; gold au 198; homocamptothecin; hydroxyurea; idarubicin hydrochloride; ifosfamide; ilmofosine; interferon alfa-2a; interferon alfa-2b; interferon alfa-nl; interferon alfa-n3; interferon beta-i a; interferon gamma-i b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peploycinsulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; rhizoxin; rhizoxin d; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; strontium chloride sr 89; sulofenur; talisomycin; taxane; taxoid; tecogalan sodium; tegafur; teloxantrone hydrochloride; temoporfin; teniposide; teroxirone; testolactone; thiamiprine; thioguanine; thiotepa; thymitaq; tiazofurin; tirapazamine; tomudex; top53; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vinblastine sulfate; vincristine; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 2-chlorodeoxyadenosine; 2′ deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-Nnitrosourea (MNU); N, N′-Bis (2-chloroethyl)-N-nitrosourea (BCNU); N-(2-chloroethyl)-N′cyclohexyl-N-nitrosourea (CCNU); N-(2-chloroethyl)-N′-(trans⁻⁴-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; cisplatin; carboplatin; ormaplatin; oxaliplatin;C1-973; DWA 2114R; JM216; JM335; Bis (platinum); tomudex; azacitidine; cytarabine; gemcitabine; 6-mercaptopurine; 6-thioguanine; hypoxanthine; teniposide 9-amino camptothecin; topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-hydroxyphenyl) retinamide; 13-cis retinoic acid; 3-methyl TTNEB; 9-cis retinoic acid; fludarabine (2-F-ara-AMP); or 2-chlorodeoxyadenosine (2-Cda).

Other therapeutic compounds that can be conjugated to, admixed with, or administered separately from an antagonistic TNFR2 single-chain polypeptide, antibody, or antigen-binding fragment thereof described herein in order to treat, prevent, or study the progression of a disease associated with aberrant cell proliferation include, but are not limited to, cytotoxic agents such as 20-pi-1,25 dihydroxyvitamin D3; 5-ethynyluracil; abiraterone; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones; epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+ estrogen+ progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; rnerbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+ pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single-chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem− cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### Labeled Anti-TNFR2 Polypeptides

In some embodiments, antagonistic TNFR2 single-chain polypeptides, antibodies, antigen-binding fragments thereof, or constructs described herein are conjugated to another molecule (e.g., an epitope tag) for the purpose of purification or detection. Examples of such molecules that are useful in protein purification include those that present structural epitopes capable of being recognized by a second molecule. This is a common strategy that is employed in protein purification by affinity chromatography, in which a molecule is immobilized on a solid support and exposed to a heterogeneous mixture containing a target protein conjugated to a molecule capable of binding the immobilized compound. Examples of epitope tag molecules that can be conjugated to antagonistic TNFR2 polypeptides for the purposes of molecular recognition include, without limitation, maltose-binding protein, glutathione-S-transferase, a poly-histidine tag, a FLAG-tag, a myc-tag, human influenza hemagglutinin (HA) tag, biotin, streptavidin. Conjugates containing the epitopes presented by these molecules are capable of being recognized by such complementary molecules as maltose, glutathione, a nickel-containing complex, an anti-FLAG antibody, an anti-myc antibody, an anti-HA antibody, streptavidin, or biotin, respectively. For example, one can purify an antagonistic TNFR2 antibody or fragment thereof described herein that has been conjugated to an epitope tag from a complex mixture of other proteins and biomolecules (e.g., DNA, RNA, carbohydrates, phospholipids, etc.) by treating the mixture with a solid phase resin containing an complementary molecule that can selectively recognize and bind the epitope tag of the antagonistic anti-TNFR2 antibody or fragment thereof. Examples of solid phase resins include agarose beads, which are compatible with purifications in aqueous solution.

An antagonistic TNFR2 polypeptide described herein can also be covalently appended to a fluorescent molecule, e.g., to detect the antibody or antigen-binding fragment thereof by fluorimetry and/or by direct visualization using fluorescence microscopy. Exemplary fluorescent molecules that can be conjugated to antibodies described herein include green fluorescent protein, cyan fluorescent protein, yellow fluorescent protein, red fluorescent protein, phycoerythrin, allophycocyanin, hoescht, 4′,6-diamidino-2-phenylindole (DAPI), propidium iodide, fluorescein, coumarin, rhodamine, tetramethylrhoadmine, and cyanine. Additional examples of fluorescent molecules suitable for conjugation to antibodies described herein are well-known in the art and have been described in detail in, e.g., U.S. Pat. Nos. 7,417,131 and 7,413,874, each of which is incorporated by reference herein.

Antagonistic TNFR2 polypeptides containing a fluorescent molecule are particularly useful for monitoring the cell-surface localization properties of antibodies and fragments thereof described herein. For instance, one can expose cultured mammalian cells (e.g., T-reg cells) to antagonistic TNFR2 polypeptides described herein that have been covalently conjugated to a fluorescent molecule and subsequently analyze these cells using conventional fluorescent microscopy techniques known in the art. Confocal fluorescent microscopy is a particularly powerful method for determining cell-surface localization of antagonistic TNFR2 polypeptides, as individual planes of a cell can be analyzed in order to distinguish antibodies or fragments thereof that have been internalized into a cell's interior, e.g., by receptor-mediated endocytosis, from those that are bound to the external face of the cell membrane. Additionally, cells can be treated with antagonistic TNFR2 antibodies conjugated to a fluorescent molecule that emits visible light of a particular wavelength (e.g., fluorescein, which fluoresces at about 535 nm) and an additional fluorescent molecule that is known to localize to a particular site on the T-reg cell surface and that fluoresces at a different wavelength (e.g., a molecule that localizes to CD25 and that fluoresces at about 599 nm). The resulting emission patterns can be visualized by confocal fluorescence microscopy and the images from these two wavelengths can be merged in order to reveal information regarding the location of the antagonistic TNFR2 antibody or antigen-binding fragment thereof on the T-reg cell surface with respect to other receptors.

Bioluminescent proteins can also be incorporated into a fusion protein for the purposes of detection and visualization of an antagonistic TNFR2 polypeptide, such as a single-chain polypeptide, antibody, or fragment thereof. Bioluminescent proteins, such as Luciferase and aequorin, emit light as part of a chemical reaction with a substrate (e.g., luciferin and coelenterazine). Exemplary bioluminescent proteins suitable for use as a diagnostic sequence and methods for their use are described in, e.g., U.S. Pat. Nos. 5,292,658, 5,670,356, 6,171,809, and 7,183,092, each of which is herein incorporated by reference. Antagonistic TNFR2 antibodies or fragments thereof labeled with bioluminescent proteins are a useful tool for the detection of antibodies described herein following an in vitro assay. For instance, the presence of an antagonistic TNFR2 antibody that has been conjugated to a bioluminescent protein can be detected among a complex mixture of additional proteins by separating the components of the mixture using gel electrophoresis methods known in the art (e.g., native gel analysis) and subsequently transferring the separated proteins to a membrane in order to perform a Western blot. Detection of the antagonistic TNFR2 polypeptide among the mixture of other proteins can be achieved by treating the membrane with an appropriate Luciferase substrate and subsequently visualizing the mixture of proteins on film using established protocols.

The polypeptides (e.g., single-chain polypeptides, antibodies, antigen-binding fragments thereof, and constructs) described herein can also be conjugated to a molecule comprising a radioactive nucleus, such that an antibody or fragment thereof described herein can be detected by analyzing the radioactive emission pattern of the nucleus. Alternatively, an antagonistic TNFR2 antibody or fragment thereof can be modified directly by incorporating a radioactive nucleus within the antibody during the preparation of the protein. Radioactive isotopes of methionine (³⁵S), nitrogen (¹⁵N), or carbon (¹³C) can be incorporated into antibodies or fragments thereof described herein by, e.g., culturing bacteria in media that has been supplemented with nutrients containing these isotopes. Optionally, tyrosine derivatives containing a radioactive halogen can be incorporated into an antagonistic TNFR2 polypeptide by, e.g., culturing bacterial cells in media supplemented with radiolabeled tyrosine. It has been shown that tyrosine functionalized with a radioactive halogen at the C2 position of the phenol system are rapidly incorporated into elongating polypeptide chains using the endogenous translation enzymes in vivo (U.S. Pat. No. 4,925,651; incorporated herein by reference). The halogens include fluorine, chlorine, bromine, iodine, and astatine. Additionally, antagonistic TNFR2 polypeptides can be modified following isolation and purification from cell culture by functionalizing polypeptides described herein with a radioactive isotope. The halogens represent a class of isotopes that can be readily incorporated into a purified protein by aromatic substitution at tyrosine or tryptophan, e.g., via reaction of one or more of these residues with an electrophilic halogen species. Examples of radioactive halogen isotopes include ¹⁸F, ⁷⁵Br, ⁷⁷Br, ¹²²I, ¹²³I, ¹²⁴I, ¹²⁵I, ¹²⁹I, ¹³¹I, or ²¹¹At.

Another alternative strategy for the incorporation of a radioactive isotope is the covalent attachment of a chelating group to the antagonistic TNFR2 polypeptide, such as a single-chain polypeptide, antibody, fragment thereof, or construct. Chelating groups can be covalently appended to an antagonistic TNFR2 antibody or fragment thereof by attachment to a reactive functional group, such as a thiol, amino group, alcohol, or carboxylic acid. The chelating groups can then be modified to contain any of a variety of metallic radioisotopes, including, without limitation, such radioactive nuclides as ¹²⁵I, ⁶⁷Ga, ¹¹¹In, ⁹⁹Tc, ¹⁶⁹Yb, ¹⁸⁶Re, ¹²³I, ¹²⁴I, ¹²⁵I, ¹³¹I, ⁹⁹mTc, ¹¹¹In, ⁶⁴Cu, ⁶⁷Cu, ¹⁸⁶Re, ¹⁸⁸Re, ¹⁷⁷Lu ⁹⁰Y, ⁷⁷As, ⁷²As, ⁸⁶Y, ⁸⁹Zr, ²¹¹At, ²¹²Bi, ²¹³Bi, or ²²⁵Ac.

In some embodiments, it may be desirable to covalently conjugate the polypeptides (e.g., single-chain polypeptides, antibodies, fragments thereof, or construct) described herein with a chelating group capable of binding a metal ion from heavy elements or rare earth ions, such as Gd³⁺, Fe³⁺, Mn³⁺, or Cr²⁺. Conjugates containing chelating groups that are coordinated to such paramagnetic metals are useful as in MRI imaging applications. Paramagnetic metals include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). In this way, antagonistic TNFR2 polypeptides can be detected by MRI spectroscopy. For instance, one can administer antagonistic TNFR2 antibodies or fragments thereof conjugated to chelating groups bound to paramagnetic ions to a mammalian subject (e.g., a human patient) in order to monitor the distribution of the antibody following administration. This can be achieved by administration of the antibody to a patient by any of the administration routes described herein, such as intravenously, and subsequently analyzing the location of the administered antibody by recording an MRI of the patient according to established protocols.

Antagonistic TNFR2 polypeptides (e.g., single-chain polypeptides, antibodies, antigen-binding fragments thereof, and constructs) can additionally be conjugated to other molecules for the purpose of improving the solubility and stability of the protein in aqueous solution. Examples of such molecules include PEG, PSA, bovine serum albumin (BSA), and human serum albumin (HSA), among others. For instance, one can conjugate an antagonistic TNFR2 polypeptide to carbohydrate moieties in order to evade detection of the antibody or fragment thereof by the immune system of the patient receiving treatment. This process of hyperglycosylation reduces the immunogenicity of therapeutic proteins by sterically inhibiting the interaction of the protein with B cell receptors in circulation. Alternatively, antagonistic TNFR2 antibodies or fragments thereof can be conjugated to molecules that prevent clearance from human serum and improve the pharmacokinetic profile of antibodies described herein. Exemplary molecules that can be conjugated to or inserted within anti-TNFR2 antibodies or fragments thereof described herein so as to attenuate clearance and improve the pharmacokinetic profile of these antibodies and fragments include salvage receptor binding epitopes. These epitopes are found within the Fc region of an IgG immunoglobulin and have been shown to bind Fc receptors and prolong antibody half-life in human serum. The insertion of salvage receptor binding epitopes into anti-TNFR2 antibodies or fragments thereof can be achieved, e.g., as described in U.S. Pat. No. 5,739,277; incorporated herein by reference.

### Modified Antagonistic TFNR2 Polypeptides

In addition to conjugation to other therapeutic agents and labels for identification or visualization, antagonistic TNFR2 polypeptides (e.g., single-chain polypeptides, antibodies, antigen-binding fragments thereof, and constructs) described herein can also be modified so as to improve their pharmacokinetic profile, biophysical stability, or inhibitory capacity. For instance, any cysteine residue not involved in maintaining the proper conformation of the antagonistic TNFR2 polypeptide may be substituted with an isosteric or isolectronic amino acid (e.g., serine) in order to improve the oxidative stability of the molecule and prevent aberrant crosslinking. Conversely, cystine bond(s) may be added to the antibody or fragment thereof to improve its stability (particularly where the antibody is an antibody fragment, such as an Fv fragment). This can be accomplished, e.g., by altering a polynucleotide encoding the antibody heavy and light chains or a polynucleotide encoding an antibody fragment so as to encode one or more additional pairs of cysteine residues that can form disulfide bonds under oxidative conditions in order to reinforce antibody tertiary structure (see, e.g., U.S. Pat. No. 7,422,899; incorporated herein by reference).

Another useful modification that may be made to antagonistic TNFR2 polypeptides (e.g., single-chain polypeptides, antibodies, antigen-binding fragments thereof, and constructs) described herein includes altering the glycosylation profile of these antibodies and fragments thereof. This can be achieved, e.g., by substituting, inserting, or deleting amino acids in an antagonistic TNFR2 antibody so as to insert or remove a glycosylation site. Glycosylation of antibodies typically occurs in N-linked or O-linked fashion. N-linked glycosylation is a process whereby the attachment of a carbohydrate moiety to an antibody occurs at the side-chain of an asparagine residue. Consensus amino acid sequences for N-linked glycosylation include the tripeptide sequences asparagine-X-serine (NXS) and asparagine-X-threonine (NXT), where X is any amino acid except proline. The insertion of either of these tripeptide sequences in a polypeptide (e.g., an antagonistic TNFR2 antibody) creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine are also competent substrates for glycoside formation. Addition of glycosylation sites to an anti-TNFR2 antibody can thus be accomplished by altering the amino acid sequence of the antibody (e.g., using recombinant expression techniques as described herein) such that it contains one or more of the above-described tripeptide sequences to promote N-linked glycosylation, or one or more serine or threonine residues to the sequence of the original antibody engender O-linked glycosylation (see, e.g., U.S. Pat. No. 7,422,899; incorporated herein by reference).

In alternative cases, it may be desirable to modify the antibody or fragment thereof described herein with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. For instance, cysteine residues may be introduced in the Fc region of an anti-TNFR2 antibody or fragment thereof (e.g., by recombinant expression techniques as described herein), so as to facilitate additional inter-chain disulfide bond formation in this region. The homodimeric antibody thus generated may have increased conformational constraint, which may foster improved internalization capability and/or increased complement-mediated cell killing and antibody-dependent cellular cytotoxicity (ADCC). Homodimeric antibodies with enhanced anti-tumor activity may also be prepared using heterobifunctional cross-linkers as described, for example, in Wolff et al. (Canc. Res., 53:2560-2565, 1993); incorporated herein by reference. Alternatively, an antibody can be engineered which has dual Fc regions and may thereby have enhanced complement lysis and ADCC capabilities (see Stevenson et al. (Anti-Canc. Drug Des., 3:219-230, 1989); incorporated herein by reference).

The serum half-life of antagonistic TNFR2 polypeptides (e.g., single-chain polypeptides, antibodies, and antigen-binding fragments thereof) described herein can be improved in some embodiments by incorporating one more amino acid modifications, such as by altering the CH1 or CL region of the Fab domain to introduce a salvage receptor motif, e.g., that found in the two loops of a CH2 domain of an Fc region of an IgG. Such alterations are described, for instance, in U.S. Pat. Nos. 5,869,046 and 6,121,022; incorporated herein by reference. Additional framework modifications can also be made to reduce immunogenicity of the antibody or fragment thereof or to reduce or remove T cell epitopes that reside therein, as described for instance in US2003/0153043; incorporated herein by reference.

### Methods of Treatment

Antagonistic TNFR2 polypeptides, such a dominant antagonistic TNFR2 polypeptide described herein, can be used to treat a patient suffering from a cell proliferation disorder (such as a cancer described herein), an infectious disease (such as a viral, bacterial, fungal, or parasitic infection described herein), or another disease mediated by TNFR2 signaling. These indications are explained in detail in the sections that follow.

### Methods of Treating Cell Proliferation Disorders

Antagonistic TNFR2 polypeptides, such as dominant antagonistic TNFR2 polypeptides (e.g., single-chain polypeptides, antibodies, antigen-binding fragments thereof, and constructs) described herein are useful therapeutics for the treatment of a wide array of cancers and cell proliferation disorders. Antagonistic TNFR2 polypeptides, such as dominant antagonistic TNFR2 polypeptides (e.g., single-chain polypeptides, antibodies, antigen-binding fragments thereof, and constructs) can be administered to a mammalian subject, such as a human, suffering from a cell proliferation disorder, such as cancer, e.g., to enhance the effectiveness of the adaptive immune response against the target cancer cells.

In particular, antagonistic TNFR2 polypeptides (e.g., single-chain polypeptides, antibodies, antigen-binding fragments thereof, and constructs) described herein can be administered to a mammalian subject, such as a human, to reduce or inhibit T-reg cell growth and activation, which allows tumor-infiltrating T lymphocytes to localize to cells presenting tumor-associated antigens and to promote cytotoxicity. In addition, polypeptides described herein may synergize with existing adoptive T cell therapy platforms, as one of the limitations on the effectiveness of this strategy has been the difficulty of prolonging cytotoxicity of tumor-reactive T cells following infusion into a mammalian subject (e.g., a human). Polypeptides described herein may also promote the activity of allogeneic T lymphocytes, which may express foreign MHC proteins and may be increasingly susceptible to inactivation by the host immune system. For example, antagonistic TNFR2 polypeptides described herein can mitigate the T-reg-mediated depletion of tumor-reactive T cells by suppressing the growth and proliferation of T-reg cells that typically accompanies T cell infusion. For instance, polypeptides (e.g., single-chain polypeptides, antibodies, antigen-binding fragments thereof, and constructs) described herein may be capable of reducing the growth of T-reg cells by about 50% to about 200% relative to untreated cells (e.g., 50%, 75%, 100%, 125%, 150%, 175%, or 200%). The reduction in cellular growth does not require the presence of TNFα. In some embodiments, polypeptides (e.g., single-chain polypeptides, antibodies, antigen-binding fragments thereof, and constructs) described herein may be capable of restricting the growth of T-reg cells in the presence of TNFα to between 90% and 150% relative to untreated cells (e.g., 90%, 100%, 110%, 120%, 130%, 140%, or 150%). Antagonistic TNFR2 polypeptides (e.g., single-chain polypeptides, antibodies, antigen-binding fragments thereof, and constructs) described herein are also capable of restricting the proliferation of T-reg cells to less than 70% (e.g., 60%, 50%, 40%, 30%, 20%, 10%, 5%, or 1%) of that of an untreated population of T-reg cells. Antagonistic TNFR2 polypeptides (e.g., single-chain polypeptides, antibodies, antigen-binding fragments thereof, and constructs) described herein are also capable of decreasing the survival of T-reg cells by about 10% (e.g., by about 20%, 30%, 40%, or 50%, or more) relative to an untreated population of T-reg cells.

Antagonistic TNFR2 polypeptides (e.g., single-chain polypeptides, antibodies, antigen-binding fragments thereof, and constructs) described herein can be administered to a mammalian subject (e.g., a human) suffering from cancer in order to improve the condition of the patient by promoting the immune response against cancer cells and tumorogenic material. Polypeptides described herein can be administered to a subject, e.g., via any of the routes of administration described herein. Polypeptides described herein can also be formulated with excipients, biologically acceptable carriers, and may be optionally conjugated to, admixed with, or co-administered separately (e.g., sequentially) with additional therapeutic agents, such as anti-cancer agents. Cancers that can be treated by administration of antibodies or antigen-binding fragments thereof described herein include such cancers as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer. Particular cancers that can be treated by administration of antibodies or antigen-binding fragments thereof described herein include, without limitation, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

For example, antagonistic TNFR2 polypeptides (e.g., single-chain polypeptides, antibodies, antigen-binding fragments thereof, and constructs) described herein can be administered to a patient (e.g., a mammalian patient, such as a human patient) in order to treat a cancer characterized by TNFR2+ cancer cells, such as Hodgkin's lymphoma, cutaneous non-Hodgkin's lymphoma, T cell lymphoma, ovarian cancer, colon cancer, multiple myeloma, renal cell carcinoma, skin cancer, lung cancer, liver cancer, endometrial cancer, a hematopoietic or lymphoid cancer, a central nervous system cancer (e.g., glioma, blastoma, or another cancer of the central nervous system described herein or known in the art), breast cancer, pancreatic cancer, stomach cancer, esophageal cancer, and upper gastrointestinal cancer.

An antagonistic TNFR2 polypeptide described herein can also be co-administered with a therapeutic antibody that exhibits reactivity towards a cancer cell. In this way, antagonistic TNFR2 polypeptides (e.g., single-chain polypeptides, antibodies, antigen-binding fragments thereof, and constructs) described herein may synergize not only with the adaptive immune response, e.g., by prolonging T lymphocyte tumor reactivity, but also with other inhibitors of tumor cell growth. Examples of additional therapeutic antibodies that can be used to treat cancer and other cell proliferation disorders include those that exhibit reactivity with a tumor antigen or a cell-surface protein that is overexpressed on the surface of a cancer cell. Exemplary antibodies that can be admixed, co-administered, or sequentially administered with antagonistic TNFR2 polypeptides described herein include, without limitation, Trastuzamb (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), ⁹⁰Y-Ibritumomab Tiuxetan (ZEVALIN®), and ¹³¹I-Tositumomab (BEXXAR®), which are described in detail in Scott et al. (Cancer Immun., 12:14-21, 2012); incorporated herein by reference.

A physician having ordinary skill in the art can readily determine an effective amount of an antagonistic TNFR2 polypeptide, such as single-chain polypeptide, antibody, antibody fragment, or construct described herein, for administration to a mammalian subject (e.g., a human) in need thereof. For example, a physician could start prescribing doses of a polypeptide described herein at levels lower than that required in order to achieve the desired therapeutic effect and gradually increase the dosage until the desired effect is achieved. Alternatively, a physician may begin a treatment regimen by administering an antagonistic TFNR2 polypeptide, such as a single-chain polypeptide, antibody, antibody fragment, or construct at a high dose and subsequently administer progressively lower doses until a therapeutic effect is achieved (e.g., a reduction in the volume of one or more tumors, a decrease in the population of T-reg cells, or remission of a cell proliferation disorder). In general, a suitable daily dose of a single-chain polypeptide, antibody, antigen-binding fragment thereof, or construct described herein will be an amount of the compound which is the lowest dose effective to produce a therapeutic effect. An antagonistic TNFR2 polypeptide described herein may be administered, e.g., by injection, such as by intravenous, intramuscular, intraperitoneal, or subcutaneous injection, optionally proximal to the site of the target tissue (e.g., a tumor). A daily dose of a therapeutic composition of an antagonistic TNFR2 polypeptide described herein may be administered as a single dose or as two, three, four, five, six or more doses administered separately at appropriate intervals throughout the day, week, month, or year, optionally, in unit dosage forms. While it is possible for an antagonistic TNFR2 polypeptide described herein to be administered alone, it may also be administered as a pharmaceutical formulation in combination with excipients, carriers, and optionally, additional therapeutic agents.

Polypeptides (e.g., single-chain polypeptides, antibodies, antigen-binding fragments thereof, and constructs) described herein can be monitored for their ability to attenuate the progression of a cell proliferation disease, such as cancer, by any of a variety of methods known in the art. For instance, a physician may monitor the response of a mammalian subject (e.g., a human) to treatment with a polypeptide, such as a single-chain polypeptide, antibody, antibody fragment, or construct described herein by analyzing the volume of one or more tumors in the patient. For example, polypeptides (e.g., single-chain polypeptides, antibodies, antigen-binding fragments thereof, and constructs) described herein may be capable of reducing tumor volume by between 1% and 100% (e.g., 1%, 5%, 10%, 20%, 30%, 40%, 50%, 60%, 70%, 80%, 90%, 95%, 99%, or 100%). Alternatively, a physician may monitor the responsiveness of a subject (e.g., a human) to treatment with antagonistic TNFR2 polypeptides, such as single-chain polypeptides, antibodies, antigen-binding fragments thereof, or constructs described herein by analyzing the T-reg cell population in the lymph of a particular subject. For instance, a physician may withdraw a sample of blood from a mammalian subject (e.g., a human) and determine the quantity or density of T-reg cells (e.g., CD4+CD25+ FOXP3+ T-reg cells or CD17+ T-reg cells) using established procedures, such as fluorescence activated cell sorting.

### Methods of Treating Infectious Diseases

Antagonistic TNFR2 polypeptides (e.g., single-chain polypeptides, antibodies, antigen-binding fragments thereof, and constructs) described herein can also be used for treating infectious diseases, such as those caused by any one or more of a virus, a bacterium, a fungus, or a parasite (e.g., a eukaryotic parasite). For instance, antagonistic TNFR2 polypeptides (e.g., single-chain polypeptides, antibodies, antigen-binding fragments thereof, and constructs) can be administered to a mammalian subject (e.g., a human) suffering from an infectious disease in order to treat the disease, as well as to alleviate one or more symptoms of the disease.

For example, antagonistic TNFR2 polypeptides (e.g., single-chain polypeptides, antibodies, antigen-binding fragments thereof, and constructs) described herein can be used for treating, or alleviating one or more symptoms of, viral infections in a mammalian subject, such as a human, that are caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parand virus, Pichinde virus, Pirital virus, Sabid virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O‘nyong’nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1 N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T lymphotropic virus Types I and II (HTLV-1 and HTLV-2, respectively); Friend Leukemia Virus; and transmissible spongiform encephalopathy, such as chronic wasting disease. Particularly, methods described herein include administering an antagonistic TNFR2 polypeptide described herein to a human in order to treat an HIV infection (such as a human suffering from AIDS).

Antagonistic TNFR2 polypeptides (e.g., single-chain polypeptides, antibodies, antigen-binding fragments thereof, and constructs) described herein can also be used for treating, or alleviating one or more symptoms of, bacterial infections in a mammalian subject (e.g., a human). Examples of bacterial infections that may be treated by administration of an antagonistic TNFR2 polypeptide, such as a single-chain polypeptide, antibody, or antibody fragment described herein include, without limitation, those caused by bacteria within the genera Streptococcus, Bacillus, Listeria, Corynebacterium, Nocardia, Neisseria, Actinobacter, Moraxella, Enterobacteriacece (e.g., E. coli, such as O157:H7), Pseudomonas (such as Pseudomonas aeruginosa), Escherichia, Klebsiella, Serratia, Enterobacter, Proteus, Salmonella, Shigella, Yersinia, Haemophilus, Bordetella (such as Bordetella pertussis), Legionella, Pasteurella, Francisella, Brucella, Bartonella, Clostridium, Vibrio, Campylobacter, Staphylococcus, Mycobacterium (such as Mycobacterium tuberculosis and Mycobacterium avium paratuberculosis, and Helicobacter (such as Helicobacter pylon and Helicobacter hepaticus). Particularly, methods described herein include administering an antagonistic TNFR2 polypeptide, such as a single-chain polypeptide, antibody, antigen-binding fragment thereof, or construct that contains one or more, or all, of the CDR sequences of TNFRAB4, such as a human, humanized, or chimeric variant of TNFRAB4, to a human or a non-human mammal in order to treat a Mycobacterium tuberculosis infection. Particular methods described herein include administering an antagonistic TNFR2 polypeptide described herein to bovine mammals or bison in order to treat a Mycobacterium tuberculosis infection. Additionally, methods described herein include administering an antagonistic TNFR2 polypeptide described herein to a human or a non-human mammal in order to treat a Mycobacterium avium paratuberculosis infection. Particular methods described herein include administering an antagonistic TNFR2 polypeptide described herein to bovine mammals or bison in order to treat a Mycobacterium avium paratuberculosis infection.

Antagonistic TNFR2 polypeptides (e.g., single-chain polypeptides, antibodies, antigen-binding fragments thereof, and constructs) described herein can also be administered to a mammalian subject (e.g., a human) for treating, or alleviating one or more symptoms of, parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites that can be treated according to the methods described herein include, without limitation, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Leishmania major, Toxoplasma gondii, Plasmodium vivax, Plasmodium ovale, Plasmodium malariae, Plasmodium falciparum, Plasmodium yoelli, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus. Additional parasitic infections that can be treated according to the methods described herein include Onchocercas volvulus.

Antagonistic TNFR2 polypeptides (e.g., single-chain polypeptides, antibodies, and antigen-binding fragments thereof) can also be administered to a mammalian subject (e.g., a human) in order to treat, or to alleviate one or more symptoms of, fungal infections. Examples of fungal infections that may be treated according to the methods described herein include, without limitation, those caused by, e.g., Aspergillus, Candida, Malassezia, Trichosporon, Fusarium, Acremonium, Rhizopus, Mucor, Pneumocystis, and Absidia. Exemplary fungal infections that can be treated according to the methods described herein also include Pneumocystis carinii, Paracoccidioides brasiliensis and Histoplasma capsulatum.

### Pharmaceutical Compositions

Pharmaceutical compositions containing an antagonistic TNFR2 polypeptide, such as a single-chain polypeptide, antibody, antigen-binding fragment thereof, or construct described herein can be prepared using methods known in the art. Pharmaceutical compositions described herein may contain an antagonistic TNFR2 polypeptide described herein in combination with one or more pharmaceutically acceptable excipients.

For instance, pharmaceutical compositions described herein can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions. The compositions can also be prepared so as to contain the active agent (e.g., an antagonistic anti-TNFR2 antibody or fragment thereof) at a desired concentration. For example, a pharmaceutical composition described herein may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent (e.g., an antagonistic TNFR2 polypeptide described herein, such as a dominant antagonistic TNFR2 polypeptide described herein) that can be incorporated into a pharmaceutical formulation can itself have a desired level of purity. For example, a polypeptide, such as a single-chain polypeptide, antibody, or antigen-binding fragment thereof described herein may be characterized by a certain degree of purity after isolating the antibody from cell culture media or after chemical synthesis, e.g., of a single-chain antibody fragment (e.g., scFv) by established solid phase peptide synthesis methods or native chemical ligation as described herein. An antagonistic TNFR2 polypeptide described herein may be at least 10% pure prior to incorporating the antibody into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions of antagonistic TNFR2 polypeptides described herein can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives must be nontoxic to the recipients at the dosages and concentrations employed.

### Buffering Agents

Buffering agents help to maintain the pH in the range which approximates physiological conditions. They can be present at concentration ranging from about 2 mM to about 50 mM. Suitable buffering agents for use with antagonistic TNFR2 polypeptides (e.g., single-chain polypeptides, antibodies, and antigen-binding fragments thereof) described herein include both organic and inorganic acids and salts thereof such as citrate buffers {e.g., monosodium citrate-disodium citrate mixture, citric acid-trisodium citrate mixture, citric acid-monosodium citrate mixture, etc.), succinate buffers {e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers {e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers {e.g., acetic acid-sodium acetate mixture, acetic acid-sodium hydroxide mixture, etc.). Additionally, phosphate buffers, histidine buffers and trimethylamine salts such as Tris can be used.

### Preservatives

Preservatives can be added to a composition described herein to retard microbial growth, and can be added in amounts ranging from 0.2%-1% (w/v). Suitable preservatives for use with antagonistic TNFR2 polypeptides (e.g., single-chain polypeptides, antibodies, antigen-binding fragments thereof, and constructs) described herein include phenol, benzyl alcohol, meta-cresol, methyl paraben, propyl paraben, octadecyldimethylbenzyl ammonium chloride, benzalconium halides {e.g., chloride, bromide, and iodide), hexamethonium chloride, and alkyl parabens such as methyl or propyl paraben, catechol, resorcinol, cyclohexanol, and 3-pentanol. Isotonifiers sometimes known as “stabilizers” can be added to ensure isotonicity of liquid compositions described herein and include polhydric sugar alcohols, for example trihydric or higher sugar alcohols, such as glycerin, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight active protein.

### Detergents

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### Other Pharmaceutical Carriers

Alternative pharmaceutically acceptable carriers that can be incorporated into a pharmaceutical composition described herein may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A composition containing an antagonistic TNFR2 antibody described herein may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### Compositions and Methods for Combination Therapy

Pharmaceutical compositions described herein may optionally include more than one active agent. For instance, compositions described herein may contain an antagonistic TNFR2 polypeptide (e.g., a single-chain polypeptide, antibody, antigen-binding fragment thereof, or construct described herein) conjugated to, admixed with, or administered separately from another pharmaceutically active molecule, e.g., a cytotoxic agent, an antibiotic, or a T lymphocyte (e.g., a gene-edited T lymphocyte for use in CAR-T therapy). For instance, an antagonistic TNFR2 polypeptide or therapeutic conjugate thereof (e.g., a drug-antibody conjugate described herein), may be admixed with one or more additional active agents that can be used to treat cancer or another cell proliferation disorder (e.g., neoplasm). Alternatively, pharmaceutical compositions described herein may be formulated for co-administration or sequential administration with one or more additional active agents that can be used to treat cancer or other cell proliferation disorders. Examples of additional active agents that can be used to treat cancer and other cell proliferation disorders and that can be conjugated to, admixed with, or administered separately from an antagonistic TNFR2 polypeptide described herein include cytotoxic agents (e.g., those described herein), as well as antibodies that exhibit reactivity with a tumor antigen or a cell-surface protein that is overexpressed on the surface of a cancer cell. Exemplary antibodies that can be conjugated to, admixed with, or administered separately from antagonistic TNFR2 antibodies described herein include, without limitation, Trastuzamb (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), ⁹⁰Y-Ibritumomab Tiuxetan (ZEVALIN®), and ¹³¹I-Tositumomab (BEXXAR®), which are described in detail in Scott et al. (Cancer Immun., 12:14-21, 2012); incorporated herein by reference.

Additional agents that can be conjugated to, admixed with, or administered separately from antagonistic TNFR2 polypeptides described herein include T lymphocytes that exhibit reactivity with a specific antigen associated with a particular pathology. For instance, antagonistic TNFR2 polypeptides described herein can be formulated for administration with a T cell that expresses a chimeric antigen receptor (CAR-T) in order to treat a cell proliferation disorder, such as a cancer described herein. Antagonistic TNFR2 polypeptides (e.g., single-chain polypeptides, antibodies, and antigen-binding fragments thereof) can synergize with CAR-T therapy by preventing T-reg cells from deactivating T lymphocytes that have been genetically modified so as to express tumor-reactive antigen receptors. In this way, CAR-T cells can be administered to a patient prior to, concurrently with, or after administration of an antagonistic TNFR2 polypeptide in order to treat a mammalian subject (e.g., a human) suffering from a cell proliferation disorder, such as cancer.

CAR-T therapy is a particularly robust platform for targeting cancer cells in view of the ability to genetically engineer T lymphocytes to express an antigen receptor specific to a tumor-associated antigen. For instance, identification of antigens overexpressed on the surfaces of tumors and other cancer cells can inform the design and discovery of chimeric T cell receptors, which are often composed of cytoplasmic and transmembrane domains derived from a naturally-occurring T cell receptor operatively linked to an extracellular scFv fragment that specifically binds to a particular antigenic peptide. T cells can be genetically modified in order to express an antigen receptor that specifically binds to a particular tumor antigen by any of a variety of genome editing techniques described herein or known in the art. Exemplary techniques for modifying a T cell genome so as to incorporate a gene encoding a chimeric antigen receptor include the CRISPER/Cas, zinc finger nuclease, TALEN, ARCUS™ platforms described herein. Methods for the genetic engineering of CAR-T lymphocytes have been described, e.g., in WO 2014/127261, WO 2014/039523, WO 2014/099671, and WO 20120790000; the disclosures of each of which are incorporated by reference herein.

CAR-T cells useful in the compositions and methods described herein include those that have been genetically modified such that the cell does not express the endogenous T cell receptor. For instance, a CAR-T cell may be modified by genome-editing techniques, such as those described herein, so as to suppress expression of the endogenous T cell receptor in order to prevent graft-versus-host reactions in a patient receiving a CAR-T infusion. Additionally, or alternatively, CAR-T cells can be genetically modified so as to reduce the expression of one or more endogenous MHC proteins. This is a particularly useful technique for the infusion of allogeneic T lymphocytes, as recognition of foreign MHC proteins represents one mechanism that promotes allograft rejection. One of skill in the art can also modify a T lymphocyte so as to suppress the expression of immune suppressor proteins, such as programmed cell death protein 1 (PD-1) and cytotoxic T lymphocyte-associated protein 4 (CTLA-4). These proteins are cell surface receptors that, when activated, attenuate T cell activation. Infusion of CAR-T cells that have been genetically modified so as to diminish the expression of one or more immunosupressor proteins represents one strategy that can be used to prolong the T lymphocyte-mediated cytotoxicity in vivo.

In addition to deleting specific genes, one can also modify CAR-T cells in order to express a T cell receptor with a desired antigen specificity. For instance, one can genetically modify a T lymphocyte in order to express a T cell receptor that specifically binds to a tumor-associated antigen in order to target infused T cells to cancer cells. An exemplary T cell receptor that may be expressed by a CAR-T cell is one that binds PD-L1, a cell surface protein that is often overexpressed on various tumor cells. As PD-L1 activates PD-1 on the surface of T lymphocytes, targeting this tumor antigen with CAR-T therapy can synergize with antagonistic TNFR2 antibodies or antibody fragments described herein in order to increase the duration of an immune response mediated by a T lymphocyte in vivo. CAR-T cells can also be modified so as to express a T cell receptor that specifically binds an antigen associated with one or more infectious disease, such as an antigen derived from a viral protein, a bacterial cell, a fungus, or other parasitic organism.

Other pharmaceutical compositions described herein include those that contain an antagonistic TNFR2 antibody or antibody fragment, interferon alpha, and/or one or more antibiotics that can be administered to a patient (e.g., a human patient) suffering from an infectious disease. For instance, an antagonistic TNFR2 antibody or antibody fragment can be conjugated to, admixed with, or administered separately from an antibiotic useful for treating one or more infectious diseases, such as amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, meropenem, cefadroxil, cefazolin, cefazlexin, cefaclor, cefoxitin, cefprozil, cefuroxime, cefdinir, cefditoren, cefoperazone, clindamycin, lincomycin, daptomycin, erythromycin, linezolid, torezolid, amoxicillin, ampicillin, bacitracin, ciprofloxacin, doxycycline, and tetracycline, among others.

### Immunotherapy Agents

An antagonistic TNFR2 polypeptide (e.g., a single-chain polypeptide, antibody, antigen-binding fragment thereof, or construct) described herein may be admixed, conjugated, administered with, or administered separately from, an immunotherapy agent, for instance, for the treatment of a cancer or infectious disease, such as a cancer or infectious disease described herein. Exemplary immunotherapy agents useful in conjunction with the compositions and methods described herein include, without limitation, an anti-CTLA-4 agent, an anti-PD-1 agent, an anti-PD-L1 agent, an anti-PD-L2 agent, an anti-TNF-α cross-linking agent, an anti-TRAIL cross-linking agent, an anti-CD27 agent, an anti-CD30 agent, an anti-CD40 agent, an anti-4-1 BB agent, an anti-GITR agent, an anti-OX40 agent, an anti-TRAILR1 agent, an anti-TRAILR2 agent, and an anti-TWEAKR agent, as well as, for example, agents directed toward the immunological targets described in Table 1 of Mahoney et al., Cancer Immunotherapy, 14:561-584 (2015), the disclosure of which is incorporated herein by reference in its entirety. For example, the immunotherapy agent may be an anti-CTLA-4 antibody or antigen-binding fragment thereof, such as ipilimumab and tremelimumab. The immunotherapy agent may be an anti-PD-1 antibody or antigen-binding fragment thereof, such as nivolumab, pembrolizumab, avelumab, durvalumab, and atezolizumab. The immunotherapy agent may be an anti-PD-L1 antibody or antigen-binding fragment thereof, such as atezolizumab or avelumab. As other examples, immunological target 4-1 BB ligand may be targeted with an anti-4-1 BB ligand antibody; immunological target OX40L may be targeted with an anti-OX40L antibody; immunological target GITR may be targeted with an anti-GITR antibody; immunological target CD27 may be targeted with an anti-CD27 antibody; immunological target TL1A may be targeted with an anti-TL1A antibody; immunological target CD40L or CD40 may be targeted with an anti-CD40L antibody; immunological target LIGHT may be targeted with an anti-LIGHT antibody; immunological target BTLA may be targeted with an anti-BTLA antibody; immunological target LAG3 may be targeted with an anti-LAG3 antibody; immunological target TIM3 may be targeted with an anti-TIM3 antibody; immunological target Singlecs may be targeted with an anti-Singlecs antibody; immunological target ICOS ligand may be targeted with an anti-ICOS ligand antibody; immunological target B7-H3 may be targeted with an anti-B7-H3 antibody; immunological target B7-H4 may be targeted with an anti-B7-H4 antibody; immunological target VISTA may be targeted with an anti-VISTA antibody; immunological target TMIGD2 may be targeted with an anti-TMIGD2 antibody; immunological target BTNL2 may be targeted with an anti-BTNL2 antibody; immunological target CD48 may be targeted with an anti-CD48 antibody; immunological target KIR may be targeted with an anti-KIR antibody; immunological target LIR may be targeted with an anti-LIR antibody; immunological target ILT may be targeted with an anti-ILT antibody; immunological target NKG2D may be targeted with an anti-NKG2D antibody; immunological target NKG2A may be targeted with an anti-NKG2A antibody; immunological target MICA may be targeted with an anti-MICA antibody; immunological target MICB may be targeted with an anti-MICB antibody; immunological target CD244 may be targeted with an anti-CD244 antibody; immunological target CSF1R may be targeted with an anti-CSF1R antibody; immunological target IDO may be targeted with an anti-IDO antibody; immunological target TGFβ may be targeted with an anti-TGFβ antibody; immunological target CD39 may be targeted with an anti-CD39 antibody; immunological target CD73 may be targeted with an anti-CD73 antibody; immunological target CXCR4 may be targeted with an anti-CXCR4 antibody; immunological target CXCL12 may be targeted with an anti-CXCL12 antibody; immunological target SIRPA may be targeted with an anti-SIRPA antibody; immunological target CD47 may be targeted with an anti-CD47 antibody; immunological target VEGF may be targeted with an anti-VEGF antibody; and immunological target neuropilin may be targeted with an anti-neuropilin antibody (see, e.g., Table 1 of Mahoney et al.).

Immunotherapy agents that may be used in conjunction with the compositions and methods described herein include, for instance, an anti-TWEAK agent, an anti− cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-HVEM agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-CD27 agent, an anti-LIGHT agent, an anti-CD137 agent, an anti-DR4 agent, an anti-CR5 agent, an anti-TNFRS agent, an anti-TNFR1 agent, an anti-FAS agent, an anti-CD95 agent, an anti-TRAIL agent, an anti-DR6 agent, an anti-EDAR agent, an anti-NGFR agent, an anti-OPG agent, an anti-RANKL agent, an anti-LTp receptor agent, an anti-BCMA agent, an anti-TACI agent, an anti-BAFFR agent, an anti-EDAR2 agent, an anti-TROY agent, and an anti-RELT agent. For instance, the immunotherapy agent may be an anti-TWEAK antibody or antigen-binding fragment thereof, an anti− cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-HVEM antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-CD27 antibody or antigen-binding fragment thereof, an anti-LIGHT antibody or antigen-binding fragment thereof, an anti-CD137 antibody or antigen-binding fragment thereof, an anti-DR4 antibody or antigen-binding fragment thereof, an anti-CR5 antibody or antigen-binding fragment thereof, an anti-TNFRS antibody or antigen-binding fragment thereof, an anti-TNFR1 antibody or antigen-binding fragment thereof, an anti-FAS antibody or antigen-binding fragment thereof, an anti-CD95 antibody or antigen-binding fragment thereof, an anti-TRAIL antibody or antigen-binding fragment thereof, an anti-DR6 antibody or antigen-binding fragment thereof, an anti-EDAR antibody or antigen-binding fragment thereof, an anti-NGFR antibody or antigen-binding fragment thereof, an anti-OPG antibody or antigen-binding fragment thereof, an anti-RANKL antibody or antigen-binding fragment thereof, an anti-LTp receptor antibody or antigen-binding fragment thereof, an anti-BCMA antibody or antigen-binding fragment thereof, an anti-TACI antibody or antigen-binding fragment thereof, an anti-BAFFR antibody or antigen-binding fragment thereof, an anti-EDAR2 antibody or antigen-binding fragment thereof, an anti-TROY antibody or antigen-binding fragment thereof, or an anti-RELT antibody or antigen-binding fragment thereof.

In some embodiments, the immunotherapy agent is an anti− cell surface lymphocyte protein antibody or antigen-binding fragment thereof, such as an antibody or antigen-binding fragment thereof that binds one or more of CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64, CD65, CD66, CD67, CD68, CD69, CD70, CD71, CD72, CD73, CD74, CD75, CD76, CD77, CD78, CD79, CD80, CD81, CD82, CD83, CD84, CD85, CD86, CD87, CD88, CD89, CD90, CD91, CD92, CD93, CD94, CD95, CD96, CD97, CD98, CD99, CD100, CD101, CD102, CD103, CD10⁴, CD10⁵, CD106, CD107, CD108, CD109, CD110, CD111, CD112, CD113, CD114, CD115, CD116, CD117, CD118, CD119, CD120, CD121, CD122, CD123, CD124, CD125, CD126, CD127, CD128, CD129, CD130, CD131, CD132, CD133, CD134, CD135, CD136, CD137, CD138, CD139, CD140, CD141, CD142, CD143, CD144, CD145, CD146, CD147, CD148, CD149, CD150, CD151, CD152, CD153, CD154, CD155, CD156, CD157, CD158, CD159, CD160, CD161, CD162, CD163, CD164, CD165, CD166, CD167, CD168, CD169, CD170, CD171, CD172, CD173, CD174, CD175, CD176, CD177, CD178, CD179, CD180, CD181, CD182, CD183, CD184, CD185, CD186, CD187, CD188, CD189, CD190, CD191, CD192, CD193, CD194, CD195, CD196, CD197, CD198, CD199, CD200, CD201, CD202, CD203, CD204, CD205, CD206, CD207, CD208, CD209, CD210, CD211, CD212, CD213, CD214, CD215, CD216, CD217, CD218, CD219, CD220, CD221, CD222, CD223, CD224, CD225, CD226, CD227, CD228, CD229, CD230, CD231, CD232, CD233, CD234, CD235, CD236, CD237, CD238, CD239, CD240, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CD293, CD294, CD295, CD296, CD297, CD298, CD299, CD300, CD301, CD302, CD303, CD304, CD305, CD306, CD307, CD308, CD309, CD310, CD311, CD312, CD313, CD314, CD315, CD316, CD317, CD318, CD319, and/or CD320.

In some embodiments, the immunotherapy agent is an agent (e.g., a polypeptide, antibody, antigen-binding fragment thereof, a single-chain polypepytide, or construct) that binds a chemokine or lymphokine, such as a chemokine or lymphokine involved in tumor growth. For instance, exemplary immunotherapy agents that may be used in conjunection with the compositions and methods described herein include agents (e.g., polypeptides, antibodies, antigen-binding fragments thereof, single-chain polypepytides, and constructs) that bind and inhibit the activity of one or more, or all, of CXCL1, CXCL2, CXCL3, CXCL8, CCL2 and CCL5. Exemplary chemokines involved in tumor growth and that may be targeted using an immunotherapy agent as described herein include those described, for instance, in Chow et al., Cancer Immunol. Res., 2:1125-1131, 2014, the disclosure of which is incorporated herein by reference. Exemplary immunotherapy agents that may be used in conjunection with the compositions and methods described herein additionally include agents (e.g., polypeptides, antibodies, antigen-binding fragments thereof, single-chain polypepytides, and constructs) that bind and inhibit the activity of one or more, or all, of CCL3, CCL4, CCL8, and CCL22, which are described, for instance, in Balkwill, Nat. Rev. Cancer, 4:540-550, 2004, the disclosure of which is incorporated herein by reference.

Additional examples of immunotherapy agents that can be used in conjunction with the compositions and methods described herein include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat.

Using the methods described herein, an antagonistic TNFR2 polypeptide (e.g., a single-chain polypeptide, antibody, antigen-binding fragment thereof, or construct) described herein may be co-administered with (e.g., admixed with) or administered separately from an immunotherapy agent. For example, an antagonistic TNFR2 polypeptide described herein (such as a single-chain polypeptide, antibody, antigen-binding fragment thereof, or construct described herein) may be administered to a patient, such as a human patient suffering from a cancer or infectious disease, simultaneously or at different times. In some embodiments, the antagonistic TNFR2 polypeptide (e.g., a single-chain polypeptide, antibody, antigen-binding fragment thereof, or construct described herein) is administered to the patient prior to administration of an immunotherapy agent to the patient. Alternatively, the antagonistic TNFR2 polypeptide (e.g., a single-chain polypeptide, antibody, antigen-binding fragment thereof, or construct described herein) may be administered to the patient after an immunotherapy agent. For example, the antagonistic TNFR2 polypeptide (e.g., a single-chain polypeptide, antibody, antigen-binding fragment thereof, or construct described herein) may be administered to the patient after a failed immunotherapy treatment. A physician of skill in the art can monitor the efficacy of immunotherapy treatment to determine whether the therapy has successfully ameliorated the pathology being treated (such as a cancer or infectious disease, e.g., a cancer or infectious disease described herein) using methods described herein and known in the art.

For instance, a physician of skill in the art may monitor the quantity of cancer cells in a sample isolated from a patient (e.g., a blood sample or biopsy sample), such as a human patient, for instance, using flow cytometry or FACS analysis. Additionally, or alternatively, a physician of skill in the art can monitor the progression of a cancerous disease in a patient, for instance, by monitoring the size of one or more tumors in the patient, for example, by CT scan, MRI, or X-ray analysis. A physician of skill in the art may monitor the progression of a cancer, such as a cancer described herein, by evaluating the quantity and/or concentration of tumor biomarkers in the patient, such as the quantity and/or concentration of cell surface-bound tumor associated antigens or secreted tumor antigens present in the blood of the patient as an indicator of tumor presence. A finding that the quantity of cancer cells, the size of a tumor, and/or the quantity or concentration of one or more tumor antigens present in the patient or in a sample isolated from the patient has not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the cancer. Based on this indication, a physician of skill in the art may administer an antagonistic TNFR2 polypeptide described herein, such as a single-chain polypeptide, antibody, antigen-binding fragment thereof, or construct described herein. Similarly, a physician a physician of skill in the art may monitor the quantity of bacterial, fungal, or parasitic cells, or the quantity of viral particles in a sample isolated from a patient suffering from an infectious disease, such as an infectious disease described herein. Additionally, or alternatively, a physician of skill in the art may monitor the progression of an infectious disease by evaluating the symptoms of a patient suffering from such a pathology. For instance, a physician may monitor the patient by determining whether the frequency and/or severity of one or more symptoms of the infectious disease have stabilized (e.g., remained the same) or decreased following treatment with an immunotherapy agent. A finding that the quantity of bacterial, fungal, or parasitic cells or viral particles in a sample isolated from the patient and/or a finding that the frequency or severity of one or more symptoms of the infectious disease have not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the infectious disease. Based on this indication, a physician of skill in the art may administer an antagonistic TNFR2 polypeptide described herein, such as a single-chain polypeptide, antibody, antigen-binding fragment thereof, or construct described herein.

### Chemotherapy Agents and Radiation Therapy

Additionally, or alternatively, an antagonistic TNFR2 polypeptide (e.g., a single-chain polypeptide, antibody, antigen-binding fragment thereof, or construct) described herein may be admixed, conjugated, administered with, or administered separately from, a chemotherapy agent, for example, for the treatment of cancer, such as a cancer described herein. Exemplary chemotherapy agents useful in conjunction with the compositions and methods described herein include, without limitation, Abiraterone Acetate, ABITREXATE® (Methotrexate), ABRAXANE® (Paclitaxel Albumin), ADRIAMYCIN®, bleomycin, vinblastine, and dacarbazine (ABVD), ADRIAMYCIN®, bleomycin, vincristine sulfate, and etoposide phosphate (ABVE), ADRIAMYCIN®, bleomycin, vincristine sulfate, etoposide phosphate, prednisone, and cyclophosphamide (ABVE-PC), doxorubicin and cyclophosphamide (AC), doxorubicin, cyclophosphamide, and paclitaxel or docetaxel (AC-T), ADCETRIS® (Brentuximab Vedotin), cytarabine, daunorubicin, and etoposide (ADE), ado-trastuzumab emtansine, ADRIAMYCIN® (doxorubicin hydrochloride), afatinib dimaleate, AFINITOR® (Everolimus), AKYNZEO® (netupitant and palonosetron hydrochloride), ALDARA® (imiquimod), aldesleukin, ALECENSA® (alectinib), alectinib, alemtuzumab, ALKERAN® for Injection (Melphalan Hydrochloride), ALKERAN® tablets (melphalan), ALIMTA® (pemetrexed disodium), ALOXI® (palonosetron hydrochloride), AMBOCHLORIN® (chlorambucil), AMBOCLORIN® (Chlorambucil), aminolevulinic acid, anastrozole, aprepitant, AREDIA® (pamidronate disodium), ARIMIDEX® (anastrozole), AROMASIN® (exemestane), ARRANON® (nelarabine), arsenic trioxide, ARZERRA® (ofatumumab), asparaginase Erwinia chrysanthemi, AVASTIN® (bevacizumab), axitinib, azacitidine, BEACOPP Becenum (carmustine), BELEODAQ® (Belinostat), belinostat, bendamustine hydrochloride, bleomycin, etoposide, and cisplatin (BEP), bevacizumab, bexarotene, BEXXAR® (tositumomab and iodine ¹³¹I tositumomab), bicalutamide, BiCNU (carmustine), bleomycin, blinatumomab, BLINCYTO® (blinatumomab), bortezomib, BOSULIF® (bosutinib), bosutinib, brentuximab vedotin, busulfan, BUSULFEX® (busulfan), cabazitaxel, cabozantinib-S-malate, CAF, CAMPATH® (alemtuzumab), CAMPTOSAR® (irinotecan hydrochloride), capecitabine, CAPOX, CARAC® (fluorouracil), carboplatin, CARBOPLATIN-TAXOL®, carfilzomib, CARMUBRIS® (carmustine), carmustine, carmustine implant, CASODEX® (bicalutamide), CEENU (lomustine), cisplatin, etoposide, and methotrexate (CEM), ceritinib, CERUBIDINE® (daunorubicin hydrochloride), CERVARIX® (recombinant HPV bivalent vaccine), cetuximab, chlorambucil, chlorambucil-prednisone, CHOP, cisplatin, CLAFEN® (cyclophosphamide), clofarabine, CLOFAREX® (clofarabine), CLOLAR® (Clofarabine), CMF, cobimetinib, cometriq (cabozantinib-S-malate), COPDAC, COPP, COPP-ABV, COSMEGEN® (dactinomycin), COTELLIC® (cobimetinib), crizotinib, CVP, cyclophosphamide, CYFOS® (ifosfamide), CYRAMZA® (ramucirumab), cytarabine, cytarabine liposome, CYTOSAR-U® (cytarabine), CYTOXAN® (cyclophosphamide), dabrafenib, dacarbazine, DACOGEN® (decitabine), dactinomycin, daratumumab, DARZALEX® (daratumumab), dasatinib, daunorubicin hydrochloride, decitabine, degarelix, denileukin diftitox, denosumab, DEPOCYT® (cytarabine liposome), dexamethasone, dexrazoxane hydrochloride, dinutuximab, docetaxel, DOXIL® (doxorubicin hydrochloride), doxorubicin hydrochloride, DOX-SL® (doxorubicin hydrochloride), DTIC-DOME® (dacarbazine), EFUDEX (fluorouracil), ELITEK® (rasburicase), ELLENCE® (epirubicin hydrochloride), elotuzumab, ELOXATIN® (oxaliplatin), eltrombopag olamine, EMEND® (aprepitant), EMPLICITI® (elotuzumab), enzalutamide, epirubicin hydrochloride, EPOCH, ERBITUX® (cetuximab), eribulin mesylate, ERIVEDGE® (vismodegib), erlotinib hydrochloride, ERWINAZE® (asparaginase Erwinia chrysanthemi), ETOPOPHOS® (etoposide phosphate), etoposide, etoposide phosphate, EVACET® (doxorubicin hydrochloride liposome), everolimus, EVISTA® (raloxifene hydrochloride), EVOMELA® (melphalan hydrochloride), exemestane, 5-FU (5-fluorouracil), FARESTON® (toremifene), FARYDAK® (panobinostat), FASLODEX® (fulvestrant), FEC, FEMARA® (letrozole), filgrastim, FLUDARA® (fludarabine phosphate), fludarabine phosphate, FLUOROPLEX® (fluorouracil), fluorouracil injection, flutamide, FOLEX® (methotrexate), FOLEX® PFS (methotrexate), FOLFIRI, FOLFIRI-bevacizumab, FOLFIRI-cetuximab, FOLFIRINOX, FOLFOX, FOLOTYN® (pralatrexate), FU-LV, fulvestrant, GARDASIL® (recombinant HPV quadrivalent vaccine), GARDASIL 9® (recombinant HPV nonavalent vaccine), GAZYVA® (obinutuzumab), gefitinib, gemcitabine hydrochloride, gemcitabine-cisplatin, gemcitabine-oxaliplatin, gemtuzumab ozogamicin, GEMZAR® (gemcitabine hydrochloride), GILOTRIF® (afatinib dimaleate), GLEEVEC® (imatinib mesylate), GLIADEL® (carmustine implant), GLIADEL® wafer (carmustine implant), glucarpidase, goserelin acetate, HALAVEN® (eribulin mesylate), HERCEPTIN® (trastuzumab), HPV bivalent vaccine, HYCAMTIN® (topotecan hydrochloride), Hyper-CVAD, IBRANCE (palbociclib), IBRITUMOMAB® tiuxetan, ibrutinib, ICE, ICLUSIG® (ponatinib hydrochloride), IDAMYCIN® (idarubicin hydrochloride), idarubicin hydrochloride, idelalisib, IFEX® (ifosfamide), ifosfamide, ifosfamidum, IL-2 (aldesleukin), imatinib mesylate, IMBRUVICA® (ibrutinib), ilmiquimod, IMLYGIC® (talimogene laherparepvec), INLYTA (axitinib), recombinant interferon alpha-2b, intron A, tositumomab, such as¹³¹I tositumomab, ipilimumab, IRESSA® (gefitinib), irinotecan hydrochloride, ISTODAX® (romidepsin), ixabepilone, ixazomib citrate, IXEMPRA® (ixabepilone), JAKAFI® (ruxolitinib phosphate), JEVTANA® (cabazitaxel), KADCYLA® (ado-trastuzumab emtansine), KEOXIFENE® (raloxifene hydrochloride), KEPIVANCE® (palifermin), KEYTRUDA® (pembrolizumab), KYPROLIS® (carfilzomib), lanreotide acetate, lapatinib ditosylate, lenalidomide, lenvatinib mesylate, LENVIMA® (lenvatinib mesylate), letrozole, leucovorin calcium, leukeran (chlorambucil), leuprolide acetate, levulan (aminolevulinic acid), LINFOLIZIN® (chlorambucil), LIPODOX® (doxorubicin hydrochloride liposome), lomustine, LONSURF® (trifluridine and tipiracil hydrochloride), LUPRON® (leuprolide acetate), LYNPARZA® (olaparib), MARQIBO® (vincristine sulfate liposome), MATULANE® (procarbazine hydrochloride), mechlorethamine hydrochloride, megestrol acetate, MEKINIST® (trametinib), melphalan, melphalan hydrochloride, mercaptopurine, MESNEX® (mesna), METHAZOLASTONE® (temozolomide), methotrexate, methotrexate LPF, MEXATE® (methotrexate), MEXATE-AQ® (methotrexate), mitomycin C, mitoxantrone hydrochloride, MITOZYTREX® (mitomycin C), MOPP, MOZOBIL® (plerixafor), MUSTARGEN® (mechlorethamine hydrochloride), MUTAMYCIN® (mitomycin C), MYLERAN® (busulfan), MYLOSAR® (azacitidine), MYLOTARG® (gemtuzumab ozogamicin), nanoparticle paclitaxel, NAVELBINE® (vinorelbine tartrate), NECITUMUMAB, nelarabine, NEOSAR® (cyclophosphamide), netupitant and palonosetron hydrochloride, NEUPOGEN® (filgrastim), NEXAVAR® (sorafenib tosylate), NILOTINIB, NINLARO® (ixazomib citrate), nivolumab, NOLVADEX® (tamoxifen citrate), NPLATE® (romiplostim), obinutuzumab, ODOMZO® (sonidegib), OEPA, ofatumumab, OFF, olaparib, omacetaxine mepesuccinate, ONCASPAR® (pegaspargase), ondansetron hydrochloride, ONIVYDE® (irinotecan hydrochloride liposome), ONTAK® (denileukin diftitox), OPDIVO® (nivolumab), OPPA, osimertinib, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, PARAPLAT® (carboplatin), PARPLATIN® (carboplatin), pazopanib hydrochloride, PCV, pegaspargase, peginterferon alpha-2b, PEG-INTRON® (peginterferon alpha-2b), pembrolizumab, pemetrexed disodium, PERJETA® (pertuzumab), pertuzumab, PLATINOL® (cisplatin), PLATINOL-AQ® (cisplatin), plerixafor, pomalidomide, POMALYST® (pomalidomide), ponatinib hydrochloride, PORTRAZZA® (necitumumab), pralatrexate, prednisone, procarbazine hydrochloride, PROLEUKIN® (aldesleukin), PROLIA® (denosumab), PROMACTA (eltrombopag olamine), PROVENGE® (sipuleucel-T), PURINETHOL® (mercaptopurine), PURIXAN® (mercaptopurine), ²²³Ra dichloride, raloxifene hydrochloride, ramucirumab, rasburicase, R—CHOP, R—CVP, recombinant human papillomavirus (HPV), recombinant interferon alpha-2b, regorafenib, R-EPOCH, REVLIMID® (lenalidomide), RHEUMATREX® (methotrexate), RITUXAN® (rituximab), rolapitant hydrochloride, romidepsin, romiplostim, rubidomycin (daunorubicin hydrochloride), ruxolitinib phosphate, SCLEROSOL® intrapleural aerosol (talc), siltuximab, sipuleucel-T, somatuline depot (lanreotide acetate), sonidegib, sorafenib tosylate, SPRYCEL® (dasatinib), STANFORD V, sterile talc powder (talc), STERITALCO (talc), STIVARGA® (regorafenib), sunitinib malate, SUTENT® (sunitinib malate), SYLATRON® (peginterferon alpha-2b), SYLVANT® (siltuximab), SYNOVIR® (thalidomide), SYNRIBO® (omacetaxine mepesuccinate), thioguanine, TAC, TAFINLAR® (dabrafenib), TAGRISSO® (osimertinib), talimogene laherparepvec, tamoxifen citrate, tarabine PFS (cytarabine), TARCEVA (erlotinib hydrochloride), TARGRETIN® (bexarotene), TASIGNA® (nilotinib), TAXOL® (paclitaxel), TAXOTERE® (docetaxel), TEMODAR® (temozolomide), temsirolimus, thalidomide, THALOMID® (thalidomide), thioguanine, thiotepa, TOLAK® (topical fluorouracil), topotecan hydrochloride, toremifene, TORISEL® (temsirolimus), TOTECT® (dexrazoxane hydrochloride), TPF, trabectedin, trametinib, TREANDA® (bendamustine hydrochloride), trifluridine and tipiracil hydrochloride, TRISENOX® (arsenic trioxide), TYKERB® (lapatinib ditosylate), UNITUXIN® (dinutuximab), uridine triacetate, VAC, vandetanib, VAMP, VARUBI® (rolapitant hydrochloride), vectibix (panitumumab), VelP, VELBAN® (vinblastine sulfate), VELCADE® (bortezomib), VELSAR (vinblastine sulfate), VEMURAFENIB, VIADUR (leuprolide acetate), VIDAZA (azacitidine), vinblastine sulfate, VINCASAR® PFS (vincristine sulfate), vincristine sulfate, vinorelbine tartrate, VIP, vismodegib, VISTOGARD® (uridine triacetate), VORAXAZE® (glucarpidase), vorinostat, VOTRIENT® (pazopanib hydrochloride), WELLCOVORIN® (leucovorin calcium), XALKORI® (crizotinib), XELODA® (capecitabine), XELIRI, XELOX, XGEVA® (denosumab), XOFIGO® (²²³Ra dichloride), XTANDI® (enzalutamide), YERVOY® (ipilimumab), YONDELIS® (trabectedin), ZALTRAP® (ziv-aflibercept), ZARXIO® (filgrastim), ZELBORAF® (vemurafenib), ZEVALIN® (ibritumomab tiuxetan), ZINECARD® (dexrazoxane hydrochloride), ziv-aflibercept, ZOFRAN® (ondansetron hydrochloride), ZOLADEX® (gGoserelin acetate), zoledronic acid, ZOLINZA® (vorinostat), ZOMETA® (zoledronic acid), ZYDELIG® (idelalisib), ZYKADIA® (ceritinib), and ZYTIGA (abiraterone acetate).

Using the methods described herein, an antagonistic TNFR2 polypeptide (e.g., a single-chain polypeptide, antibody, antigen-binding fragment thereof, or construct) described herein may be co-administered with (e.g., admixed with) or administered separately from a chemotherapy agent for the treatment of cancer. For example, an antagonistic TNFR2 polypeptide described herein (such as a single-chain polypeptide, antibody, antigen-binding fragment thereof, or construct described herein) may be administered to a patient, such as a human patient suffering from a cancer, simultaneously or at different times. In some embodiments, the antagonistic TNFR2 polypeptide (e.g., a single-chain polypeptide, antibody, antigen-binding fragment thereof, or construct described herein) is administered to the patient prior to administration of a chemotherapy agent to the patient. Alternatively, the antagonistic TNFR2 polypeptide (e.g., a single-chain polypeptide, antibody, antigen-binding fragment thereof, or construct described herein) may be administered to the patient after a chemotherapy agent. For example, the antagonistic TNFR2 polypeptide (e.g., a single-chain polypeptide, antibody, antigen-binding fragment thereof, or construct described herein) may be administered to the patient after a failed chemotherapy treatment. A physician of skill in the art can monitor the efficacy of chemotherapy treatment to determine whether the therapy has successfully ameliorated the pathology being treated (such as a cancer described herein) using methods described herein and known in the art.

For instance, a physician of skill in the art may monitor the quantity of cancer cells in a sample isolated from a patient (e.g., a blood sample or biopsy sample), such as a human patient, for instance, using flow cytometry or FACS analysis. Additionally, or alternatively, a physician of skill in the art can monitor the progression of a cancerous disease in a patient, for instance, by monitoring the size of one or more tumors in the patient, for example, by CT scan, MRI, or X-ray analysis. A physician of skill in the art may monitor the progression of a cancer, such as a cancer described herein, by evaluating the quantity and/or concentration of tumor biomarkers in the patient, such as the quantity and/or concentration of cell surface-bound tumor associated antigens or secreted tumor antigens present in the blood of the patient as an indicator of tumor presence. A finding that the quantity of cancer cells, the size of a tumor, and/or the quantity or concentration of one or more tumor antigens present in the patient or a sample isolated from the patient has not decreased, for instance, by a statistically significant amount following administration of the chemotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the chemotherapy treatment has failed to ameliorate the cancer. Based on this indication, a physician of skill in the art may administer an antagonistic TNFR2 polypeptide described herein, such as a single-chain polypeptide, antibody, antigen-binding fragment thereof, or construct described herein.

Additionally, or alternatively, an antagonistic TNFR2 polypeptide (e.g., a single-chain polypeptide, antibody, antigen-binding fragment thereof, or construct) described herein may be administered simultaneously with, or administered separately from, radiation therapy. For instance, a physician of skill in the art may administer radiation therapy to a patient, such as a human patient suffering from a cancer described herein, by treating the patient with external and/or internal electromagnetic radiation. The energy delivered by such radiation, which is typically in the form of X-rays, gamma rays, and similar forms of low-wavelength energy, can cause oxidative damage to the DNA of cancer cells, thereby leading to cell death, for instance, by apoptosis. External radiation therapy can be administered, for instance, using machinery such as a radiation beam to expose the patient to a controlled pulse of electromagnetic radiation. Additionally, or alternatively, the patient may be administered internal radiation, for instance, by administering to the patient a therapeutic agent that contains a radioactive substituent, such as agents that contain ²²³Ra or ¹³¹I, which emit high-energy alpha and beta particles, respectively. Exemplary therapeutic agents that may be conjugated to a radiolabel include, for example, small molecule chemotherapeutics, antibodies, and antigen-binding fragments thereof, among others. For instance, an antagonistic TNFR2 polypeptide (e.g., a single-chain polypeptide, antibody, antigen-binding fragment thereof, or construct) described herein may be conjugated to a radioactive substituent or a moiety that ligate such a substituent, for example, using bond-forming techniques known in the art or described herein. Such conjugates can be administered to the subject in order to deliver a therapeutic dosage of radiation therapy and a TNFR2 antagonist described herein in a simultaneous administration (see, for example, “Antagonistic TNFR2 polypeptide conjugates,” above).

In some embodiments, the antagonistic TNFR2 polypeptide (e.g., a single-chain polypeptide, antibody, antigen-binding fragment thereof, or construct described herein) is administered to the patient after failed radiation treatment. A physician of skill in the art can monitor the efficacy of radiation treatment to determine whether the therapy has successfully ameliorated the pathology being treated (such as a cancer described herein) using, e.g., methods described herein. For instance, a physician of skill in the art may monitor the quantity of cancer cells in a sample isolated from a patient (e.g., a blood sample or biopsy sample), such as a human patient, for instance, using flow cytometry or FACS analysis. Additionally, or alternatively, a physician of skill in the art can monitor the progression of a cancerous disease in a patient, for instance, by monitoring the size of one or more tumors in the patient, for example, by CT scan, MRI, or X-ray analysis. A physician of skill in the art may monitor the progression of a cancer, such as a cancer described herein, by evaluating the quantity and/or concentration of tumor biomarkers in the patient, such as the quantity and/or concentration of cell surface-bound tumor associated antigens or secreted tumor antigens present in the blood of the patient as an indicator of tumor presence. A finding that the quantity of cancer cells, the size of a tumor, and/or the quantity or concentration of one or more tumor antigens present in the patient or a sample isolated from the patient has not decreased, for instance, by a statistically significant amount following administration of the radiation therapy within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the radiation treatment has failed to ameliorate the cancer. Based on this indication, a physician of skill in the art may administer an antagonistic TNFR2 polypeptide described herein, such as a single-chain polypeptide, antibody, antigen-binding fragment thereof, or construct described herein.

In some embodiments, a physician of skill in the art may administer to a patient suffering from cancer a chemotherapeutic agent, radiation therapy, and a TNFR2 antagonist described herein (such as a single-chain polypeptide, antibody, antigen-binding fragment thereof, or construct described herein). The TNFR2 antagonist described herein, chemotherapeutic agent, and radiation therapy may be administered to the patient simultaneously (for instance, in a single pharmaceutical composition or as multiple compositions administered to the patient at the same time) or at different times. In some embodiments, the TNFR2 antagonist (such as an antibody, antigen-binding fragment thereof, single-chain polypeptide, or construct described herein) is administered to the patient first, and the chemotherapeutic agent and radiation therapy follow. Alternatively, the TNFR2 antagonist (such as a single-chain polypeptide, antibody, antigen-binding fragment thereof, or construct described herein) may be administered to the patient following chemotherapy and radiation treatment. For example, the antagonistic TNFR2 polypeptide (e.g., a single-chain polypeptide, antibody, antigen-binding fragment thereof, or construct described herein) may be administered to the patient after failed chemotherapy and/or radiation treatment.

A physician of skill in the art can monitor the efficacy of chemotherapy and radiation treatment to determine whether the therapy has successfully ameliorated the pathology being treated (such as a cancer described herein) using methods described herein, such as the methods described above. For instance, a physician of skill in the art may monitor the quantity of cancer cells in a sample isolated from a patient (e.g., a blood sample or biopsy sample), such as a human patient, for instance, using flow cytometry or FACS analysis. Additionally, or alternatively, a physician of skill in the art can monitor the progression of a cancerous disease in a patient, for instance, by monitoring the size of one or more tumors in the patient, for example, by CT scan, MRI, or X-ray analysis. A physician of skill in the art may monitor the progression of a cancer, such as a cancer described herein, by evaluating the quantity and/or concentration of tumor biomarkers in the patient, such as the quantity and/or concentration of cell surface-bound tumor associated antigens or secreted tumor antigens present in the blood of the patient as an indicator of tumor presence and even measure serum soluble TNFR2. One skilled in the art would expect a decrease in the number of activated T-regs, and increase in the numbers of T effectors and a decrease in the total number of cancer cells. Because of the specificity of these TNFR2 antibodies for cancer, the clinical monitoring would be expected to be most dramatic in the tumor microenvironment. A finding that the quantity of cancer cells, the size of a tumor, and/or the quantity or concentration of one or more tumor antigens present in the patient or a sample isolated from the patient has not decreased, for instance, by a statistically significant amount following administration of the chemotherapy agent and radiation within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the chemotherapy and radiation treatment has failed to ameliorate the cancer. Based on this indication, a physician of skill in the art may administer an antagonistic TNFR2 polypeptide described herein, such as a single-chain polypeptide, antibody, antigen-binding fragment thereof, or construct described herein.

### Blood-Brain Barrier Penetration

In certain embodiments, antagonistic TNFR2 polypeptides (e.g., single-chain polypeptides, antibodies, antigen-binding fragments thereof, and constructs) described herein can be formulated to ensure proper distribution in vivo. For example, the blood-brain barrier (BBB) excludes many highly hydrophilic compounds. To ensure that the therapeutic compositions described herein cross the BBB (if desired), they can be formulated, for example, in liposomes. Methods of manufacturing liposomes have been described, e.g., U.S. Pat. Nos. 4,522,811; 5,374,548; and 5,399,331. The liposomes may comprise one or more moieties that are selectively transported into specific cells or organs, thereby enhancing targeted drug delivery (see, e.g., V. V. Ranade (J. Clin. Pharmacol. 29:685, 1989)). Exemplary targeting moieties include, e.g., folate or biotin (see, e.g., U.S. Pat. No. 5,416,016); mannosides (Umezawa et al. (Biochem. Biophys. Res. Commun. 153:1038, 1988)); antibodies (P. G. Bloeman et al. (FEBS Lett. 357:140, 1995); M. Owais et al. (Antimicrob. Agents Chemother. 39:180, 1995)); surfactant protein A receptor (Briscoe et al. (Am. J. Physiol. 1233:134, 1995)); the disclosures of each of which are incorporated herein by reference.

### Routes of Administration and Dosing

Antagonistic TNFR2 polypeptides (e.g., single-chain polypeptides, antibodies, antigen-binding fragments thereof, and constructs) described herein can be administered to a mammalian subject (e.g., a human) by a variety of routes such as orally, transdermally, subcutaneously, intranasally, intravenously, intramuscularly, intraocularly, intratumorally, parenterally, topically, intrathecally and intracerebroventricularly. The most suitable route for administration in any given case will depend on the particular polypeptide administered, the patient, pharmaceutical formulation methods, administration methods (e.g., administration time and administration route), the patient's age, body weight, sex, severity of the diseases being treated, the patient's diet, and the patient's excretion rate.

The effective dose of an antagonistic TNFR2 polypeptide described herein can range, for instance, from about 0.0001 to about 100 mg/kg of body weight per single (e.g., bolus) administration, multiple administrations or continuous administration (e.g., a continuous infusion), or to achieve a serum concentration of 0.0001-5000 pg/mL serum concentration per single (e.g., bolus) administration, multiple administrations or continuous administration (e.g., continuous infusion), or any effective range or value therein depending on the condition being treated, the route of administration and the age, weight, and condition of the subject. In certain embodiments, e.g., for the treatment of cancer, each dose can range from about 0.0001 mg to about 500 mg/kg of body weight. For instance, a pharmaceutical composition described herein may be administered in a daily dose in the range of 0.001-100 mg/kg (body weight). The dose may be administered one or more times (e.g., 2-10 times) per day, week, month, or year to a mammalian subject (e.g., a human) in need thereof.

Antagonistic TNFR2 polypeptides described herein (e.g., single-chain polypeptides, antibodies, antigen-binding fragments thereof, and constructs) can be administered to a patient by way of a continuous intravenous infusion or as a single bolus administration. The antagonistic TNFR2 polypeptides described herein (e.g., e.g., single-chain polypeptides, antibodies, antigen-binding fragments thereof, and constructs) may be administered to a patient in an amount of, for example, from 0.01 pg to about 5 g in a volume of, for example, from 10 μL to 10 mL. The antagonistic TNFR2 polypeptides may be administered to a patient over the course of several minutes to several hours. For example, the antagonistic TNFR2 polypeptides described herein may be administered to a patient over the course of from 5 minutes to 5 hours, such as over the course of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 80 minutes, 90 minutes, 95 minutes, 100 minutes, 10⁵ minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, or 300 minutes, or more.

Antagonistic TNFR2 polypeptides (e.g., single-chain polypeptides, antibodies, antigen-binding fragments thereof, and constructs) described herein may be administered in combination with an immunotherapy agent, such as an anti-PD-1 antibody or antigen-binding fragment thereof, an anti-PD-L1 antibody or antigen-binding fragment thereof, and/or an anti-CTLA-4 antibody or antigen-binding fragment thereof. Exemplary anti-PD-1 antibodies include nivolumab, pembrolizumab, avelumab, durvalumab, and atezolizumab. Exemplary anti-CTLA4 antibodies include ipilimumab and tremelimumab. Exemplary anti-PD-L1 antibodies include atezolizumab and avelumab.

When an anti-PD-1 antibody or antigen-binding fragment thereof is administered to a patient (e.g., a patient having cancer or an infectious disease described herein) in combination with an antagonist TNFR2 polypeptide, the anti-PD-1 antibody may be administered to the patient by way of a single bolus administration or continuous intravenous infusion. For example, pembrolizumab may be administered to a human patient by way of a continuous intravenous infusion of 200 mg over the course of 30 minutes, for instance, every three weeks, as needed (KEYTRUDA® (pembrolizumab) [package insert]. Merck Sharp & Dohme Corp., Whitehouse Station, NJ, the disclosure of which is incorporated herein by reference in its entirety). In another example, nivolumab may be administered to a patient by way of a continuous intravenous infusion of 240 mg over the course of 30 minutes, for instance, every two weeks as needed. Alternatively, nivolumab may be administered to a patient by way of a continuous intravenous infusion of 480 mg over the course of 30 minutes, for instance, every four weeks as needed (OPDIVO® (nivolumab) [package insert]. Bristol-Myers Squibb Company, Princeton, NJ, the disclosure of which is incorporated herein by reference in its entirety).

When an anti-CTLA-4 antibody or antigen-binding fragment thereof is administered to a patient (e.g., a patient having cancer or an infectious disease described herein) in combination with an antagonist TNFR2 polypeptide, the anti-CTLA-4 antibody may be administered to the patient by way of a single bolus administration or continuous intravenous infusion. For example, ipilimumab may be administered to a human patient by way of a continuous intravenous infusion of 3 mg/kg over the course of 90 minutes, for instance, every three weeks, as needed, or by way of a continuous intravenous infusion of 10 mg/kg over the course of 90 minutes every three weeks for four doses, followed by 10 mg/kg over the course of 90 minutes every 12 weeks for up to 3 years (YERVOY® (ipilimumab) [package insert]. Bristol-Myers Squibb Company, Princeton, NJ, the disclosure of which is incorporated herein by reference in its entirety).

When TNFR2 antagonist polypeptides (e.g., single-chain polypeptides, antibodies, antigen-binding fragments thereof, and constructs) are administered to a patient in combination with an immunotherapy agent, such as an anti-PD-1 antibody or anti-CTLA-4 antibody, the antagonist TNFR2 polypeptide and the immunotherapy agent may be co-administered to the patient, for example, by way of a continuous intravenous infusion or bolus administration of the first agent, followed by a continuous intravenous infusion or bolus administration of the second agent. The administration of the two agents may occur concurrently. Alternatively, the administration of the antagonist TNFR2 antibody or antigen-binding fragment thereof may precede or follow the administration of the immunotherapy agent. In some embodiments, administration of the second agent (e.g., the antagonist TNFR2 polypeptide) commences within from about 5 minutes to about 4 weeks, or more, of the end of the administration of the first agent (e.g., the immunotherapy agent). For example, administration of the second agent may commence within about 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, or more, of the end of the administration of the first agent.

Therapeutic compositions can be administered with medical devices known in the art. For example, in an embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in conjunction with the compositions and methods described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### Kits Containing Antagonistic Anti-TNFR2 Polypeptides

Also included herein are kits that contain antagonistic TNFR2 polypeptides (e.g., single-chain polypeptides, antibodies, antigen-binding fragments thereof, and constructs). The kits provided herein may contain any of the antagonistic TNFR2 polypeptides described above, as well as any of the polynucleotides encoding these polypeptides, vectors containing these polynucleotides, or cells engineered to express and secrete antibodies described herein (e.g., prokaryotic or eukaryotic cells). A kit described herein may include reagents that can be used to produce the compositions described herein (e.g., antagonistic TNFR2 polypeptides, such as single-chain polypeptides, antibodies, constructs, conjugates containing antagonistic TNFR2 polypeptides, polynucleotides encoding antagonistic anti-TNFR2 polypeptides, vectors containing these polynucleotides). Optionally, kits described herein may include reagents that can induce the expression of antagonistic TNFR2 polypeptides within cells (e.g., mammalian cells), such as doxycycline or tetracycline. In other cases, a kit described herein may contain a compound capable of binding and detecting a fusion protein that contains an antagonistic TNFR2 antibody and an epitope tag. For instance, in such cases a kit described herein may contain maltose, glutathione, a nickel-containing complex, an anti-FLAG antibody, an anti-myc antibody, an anti-HA antibody, biotin, or streptavidin.

Kits described herein may also include reagents that are capable of detecting an antagonistic TNFR2 polypeptide (e.g., single-chain polypeptide, antibody, fragment thereof, or construct) directly. Examples of such reagents include secondary antibodies that selectively recognize and bind particular structural features within the Fc region of an anti-TNFR2 antibody described herein. Kits described herein may contain secondary antibodies that recognize the Fc region of an antagonistic TNFR2 antibody and that are conjugated to a fluorescent molecule. These antibody-fluorophore conjugates provide a tool for analyzing the localization of antagonistic anti-TNFR2 antibodies, e.g., in a particular tissue or cultured mammalian cell using established immunofluorescence techniques. In some embodiments, kits described herein may include additional fluorescent compounds that exhibit known sub− cellular localization patterns. These reagents can be used in combination with another antibody-fluorophore conjugate, e.g., one that specifically recognizes a different receptor on the cell surface in order to analyze the localization of an anti-TNFR2 antibody relative to other cell-surface proteins.

Kits described herein may also contain a reagent that can be used for the analysis of a patient's response to treatment by administration of antagonistic TNFR2 polypeptides (e.g., single-chain polypeptides, antibodies, antigen-binding fragments thereof, and constructs) described herein. For instance, kits described herein may include an antagonistic TNFR2 antibody and one or more reagents that can be used to determine the quantity of T-reg cells in a blood sample withdrawn from a subject (e.g., a human) that is undergoing treatment with an antibody described herein. Such a kit may contain, e.g., antibodies that selectively bind cell-surface antigens presented by T-reg cells, such as CD4 and CD25. Optionally, these antibodies may be labeled with a fluorescent dye, such as fluorescein or tetramethylrhodamine, in order to facilitate analysis of T-reg cells by fluorescence-activated cell sorting (FACS) methods known in the art. Kits described herein may optionally contain one or more reagents that can be used to quantify tumor-reactive T lymphocytes in order to determine the effectiveness of an antagonistic TNFR2 polypeptide described herein in restoring tumor-infiltrating lymphocyte proliferation. For instance, kits described herein may contain an antibody that selectively binds cell-surface markers on the surface of a cytotoxic T cell, such as CD8 or CD3. Optionally, these antibodies may be labeled with fluorescent molecules so as to enable quantitation by FACS analysis.

A kit described herein may also contain one or more reagents useful for determining the affinity and selectivity of an antagonistic TNFR2 polypeptide described herein for one or more peptides derived from TNFR2 (e.g., a peptide containing the sequence of any one of SEQ ID NOs: 31-33). For instance, a kit may contain an antagonistic TNFR2 polypeptide and one or more reagents that can be used in an ELISA assay to determine the K_{D} of an antibody described herein for one or more peptides that present a TNFR2 epitope in a conformation similar to that of the epitope in the native protein. A kit may contain, e.g., a microtiter plate containing wells that have been previously conjugated to avidin, and may contain a library of TNFR2-derived peptides, each of which conjugated to a biotin moiety. Such a kit may optionally contain a secondary antibody that specifically binds to the Fc region of an antagonistic TNFR2 antibody described herein, and the secondary antibody may be conjugated to an enzyme (e.g., horseradish peroxidase) that catalyzes a chemical reaction that results in the emission of luminescent light.

Kits described herein may also contain antagonistic TNFR2 polypeptides described herein and reagents that can be conjugated to such an antibody, including those previously described (e.g., a cytotoxic agent, a fluorescent molecule, a bioluminescent molecule, a molecule containing a radioactive isotope, a molecule containing a chelating group bound to a paramagnetic ion, etc.). These kits may additionally contain instructions for how the conjugation of an antagonistic TNFR2 antibody described herein to a second molecule, such as those described above, can be achieved.

A kit described herein may also contain a vector containing a polynucleotide that encodes an antagonistic TNFR2 polypeptide, such as any of the vectors described herein. Alternatively, a kit may include mammalian cells (e.g., CHO cells) that have been genetically altered to express and secrete antagonistic TNFR2 antibodies or fragments thereof from the nuclear genome of the cell. Such a kit may also contain instructions describing how expression of the antagonistic TNFR2 antibody or fragment thereof from a polynucleotide can be induced, and may additionally include reagents (such as, e.g., doxycycline or tetracycline) that can be used to promote the transcription of these polynucleotides. Such kits may be useful for the manufacture of antagonistic TNFR2 antibodies or antigen-binding fragments thereof described herein.

Other kits described herein may include tools for engineering a prokaryotic or eukaryotic cell (e.g., a CHO cell or a BL21(DE3) E. coli cell) so as to express and secrete an antagonistic TNFR2 polypeptide described herein from the nuclear genome of the cell. For example, a kit may contain CHO cells stored in an appropriate media and optionally frozen according to methods known in the art. The kit may also provide a vector containing a polynucleotide that encodes a nuclease (e.g., such as the CRISPER/Cas, zinc finger nuclease, TALEN, ARCUS™ nucleases described herein) as well as reagents for expressing the nuclease in the cell. The kit can additionally provide tools for modifying the polynucleotide that encodes the nuclease so as to enable one to alter the DNA sequence of the nuclease in order to direct the cleavage of a specific target DNA sequence of interest. Examples of such tools include primers for the amplification and site-directed mutagenesis of the polynucleotide encoding the nuclease of interest. The kit may also include restriction enzymes that can be used to selectively excise the nuclease-encoding polynucleotide from the vector and subsequently re-introduce the modified polynucleotide back into the vector once the user has modified the gene. Such a kit may also include a DNA ligase that can be used to catalyze the formation of covalent phosphodiester linkages between the modified nuclease-encoding polynucleotide and the target vector. A kit described herein may also provide a polynucleotide encoding an antagonistic TNFR2 polypeptide, as well as a package insert describing the methods one can use to selectively cleave a particular DNA sequence in the genome of the cell in order to incorporate the polynucleotide encoding an antagonistic TNFR2 antibody into the genome at this site. Optionally, the kit may provide a polynucleotide encoding a fusion protein that contains an antagonistic TNFR2 antibody or fragment thereof and an additional polypeptide, such as, e.g., those described herein.

### EXAMPLES

The following examples are put forth so as to provide those of ordinary skill in the art with a description of how the compositions and methods claimed herein are performed, made, and evaluated, and are intended to be purely exemplary described herein and are not intended to limit the scope of what the inventor regards as her invention.

### Example 1. Mapping the Discrete Epitopes within TNFR2 that Interact with TNFRAB4

Libraries of linear, cyclic, and bicyclic peptides derived from human TNFR2 were screened for distinct sequences within the protein that exhibit high affinity for TNFR2 antibody TNFRAB4. In order to screen conformational epitopes within TFNR2, peptides from distinct regions of the primary protein sequence were conjugated to one another to form chimeric peptides. These peptides contained cysteine residues at strategic positions within their primary sequences. This facilitated an intramolecular cross-linking strategy that was used to constrain individual peptides to a one of a wide array of three dimensional conformations. Unprotected thiols of cysteine residues were cross-linked via nucleophilic substitution reactions with divalent and trivalent electrophiles, such as 2,6-bis(bromomethyl)pyridine and 1,3,5-tris(bromomethyl)benzene, so as to form conformationally restricted cyclic and bicyclic peptides, respectively. In this way, peptides containing unique combinations of amino acids from disparate regions of the TNFR2 primary sequence were constrained so as to structurally pre-organize epitopes that may resemble those presented in the native TNFR2 tertiary structure. Libraries containing these peptides were screened by immobilizing peptides to distinct regions of a solid surface and treating the surface in turn with TNFRAB4, secondary antibody conjugated to horseradish peroxidase (HRP), and HRP substrate (2,2′-azino-di-3-ethylbenzthiazoline sulfonate) in the presence of hydrogen peroxide. The solid surface was washed in between treatment with successive reagents so as to remove excess or non-specifically bound materials. The luminescence of each region of each surface was subsequently analyzed using a charge coupled device (CCD)-camera and an image processing system.

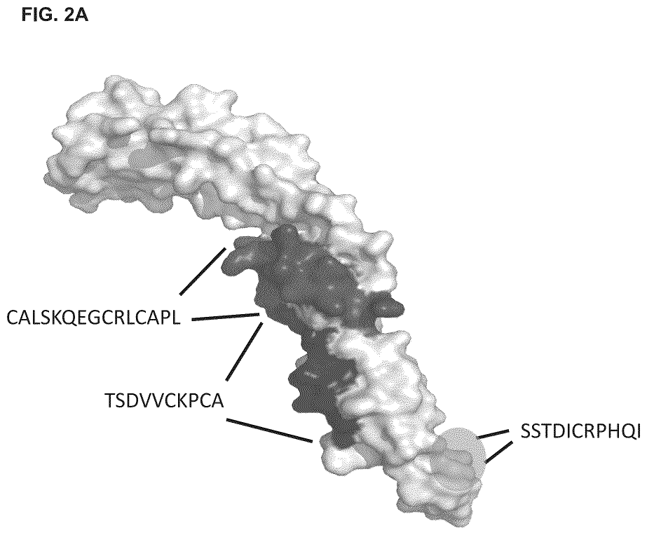

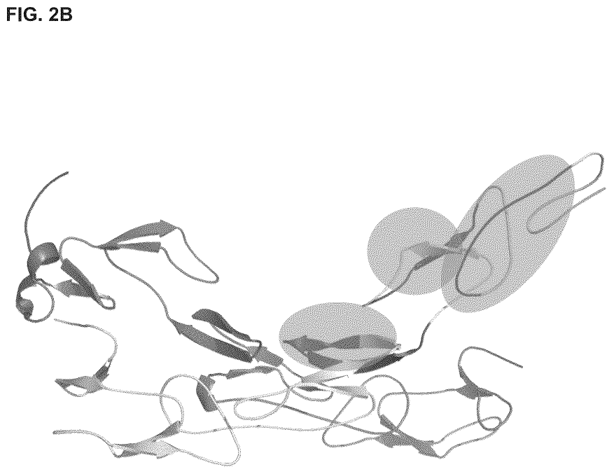

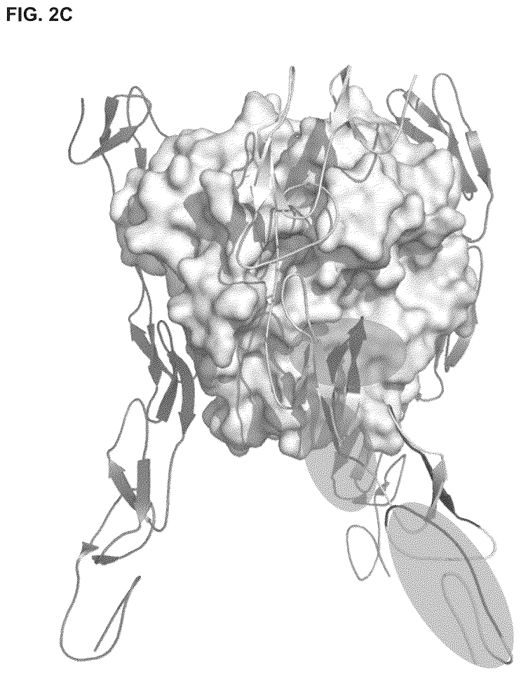

The “Constrained Libraries of Peptides on Surfaces” (CLIPS) platform starts with the conversion of the target protein, e.g., TNFR2, into a library of up to 10,000 overlapping peptide constructs, using a combinatorial matrix design (Timmerman et al., J. Mol. Recognit., 20: 283-29, 2007). On a solid carrier, a matrix of linear peptides is synthesized, which are subsequently shaped into spatially defined CLIPS constructs. Constructs representing multiple parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail. The raw luminescence data obtained from these ELISA experiments informed the analysis of epitopes present on the surface of TNFR2 that bind antagonistic TNFR2 antibodies. Structural models of TNFR2 illustrating epitopes that bind such antibodies are shown in FIGS. 2A-2C.

### Peptide Synthesis

To reconstruct epitopes of the target molecule a library of peptides was synthesized. An amino functionalized polypropylene support was obtained by grafting a proprietary hydrophilic polymer formulation via reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS® liquid handling stations (Perkin Elmer). CLIPS technology allows one to structure peptides into single loops, double-loops, triple loops, sheet-like folds, helix-like folds and combinations thereof. CLIPS templates are coupled to cysteine residues. The side-chains of multiple cysteines in the peptides are coupled to one or two CLIPS templates. For example, a 0.5 mM solution of the CLIPS template (2,6-bis(bromomethyl)pyridine) is dissolved in ammonium bicarbonate (20 mM, pH 7.8)/acetonitrile (1:3(v/v)). This solution is added to a surface-bound peptide array. The CLIPS template will react with side-chains of two cysteines as present in the solid-phase bound peptides of the peptide-arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

### Analysis of Binding Affinities of Antagonistic TNFR2 Antibodies by Surface Plasmon Resonance

The affinities of antagonistic TNFR2 antibodies for recombinant human TNFR2 were measured using BIACORE™ Analysis Services (Precision Antibody). Briefly, the antibody was biotinylated at a 5:1 stoichiometric ratio using biotinyl-LC-LC-NOSE (Thermo-Fisher) in PBS. Excess biotinylation reagent was removed by centrifugation chromatography and the biotinylated antibody was captured on 3000 RU of streptavidin surface to a level of 100 RU. Theoretical maximum of signal with TNFR2 with that level of antibody capture was 26 RU and that signal was reached with a preliminary experiment using 500 nM TNFR2 in the running buffer. Analysis of the kinetics of antigen binding was performed at a flow of 60 μL/min with 2 min injections. Antibodies were injected at a concentration of 1 mg/ml to the final capture of 100 RU. The instrument used was BIACORE™ 3000 with the BioCap chip (GE Healthcare). Double reference method was used for analysis. Reference channel contained the identical level of streptavidin.

### ELISA Screening

The binding of antibody to each of the synthesized peptides was tested in an ELISA format. Surface-immobilized peptide arrays were incubated with primary antibody solution (overnight at 4° C.). After washing, the peptide arrays were incubated with a 1/1000 dilution of an appropriate antibody peroxidase conjugate (SBA) for one hour at 25° C. After washing, the peroxidase substrate 2,2′-azino-di-3-ethylbenzthiazoline sulfonate (ABTS) and 2 μl/ml of 3 percent H₂O₂ were added. After one hour, the color development was measured. The color development was quantified with a charge coupled device (CCD) —camera and an image processing system. The values obtained from the CCD camera range from 0 to 3000 mAU, similar to a standard 96-well plate ELISA-reader. The results are quantified and stored into the Peplab database. Occasionally a well contains an air-bubble resulting in a false-positive value, the cards are manually inspected and any values caused by an air-bubble are scored as 0.

To verify the quality of the synthesized peptides, a separate set of positive and negative control peptides was synthesized in parallel. These were screened with a negative control, antibody 57.9, an antibody that does not specifically bind TNFR2 (Posthumus et al. (J. Virology. 64:3304-3309, 1990)).

Peptides that were found to bind TNFRAB4 with high affinity contain residues within TNFR2 that are structurally configured into epitopes that are preferentially bound by TNFRAB4.

### Epitope Mapping

ELISA was also used to determine linear epitopes present on the extracellular surface of TNFR2. Linear peptides corresponding to various regions within the TNFR2 primary sequence were purchased from GenScript (Piscataway, NJ), diluted in coating buffer and placed on Immulon 4HBX Flat Bottom Microtiter Plates (Thermo Scientific) at a concentration of 1 μg/well. Primary TNFR2 antagonistic antibodies (0.1 pg/well) were incubated with substrates. Secondary antibodies against rodent IgG were used to detect the primary antibodies. Absorbance was measured using the SPECTRAMAX® 190 Absorbance Plate Reader and analyzed with SoftMax Pro 6.3 (Molecular Devices).

Results of the epitope mapping analysis are shown in FIG. 1, which displays the primary structure of human TNFR2, and in FIGS. 2A-2C, which shows the three-dimensional structure of human TNFR2 as a monomer (FIG. 2A), an anti-parallel dimer (FIG. 2B), and a trimer (FIG. 2C).

### Example 2. Antagonistic TNFR2 Polypeptides that Bind Specific Epitopes within TNFR2 Kill T-Reg Cells, Expand T Effector Cells, and Deplete Cancer Cells

Antagonistic TFNR2 polypeptides, such as antibodies, antigen-binding fragments thereof, single-chain polypeptides, and constructs thereof, described herein, exhibit the ability to kill T-reg cells, induce the proliferation of T effector cells, and kill TNFR2-expressing cancer cells in the tumor microenvironment. To further investigate these activities, a series of 27 TNFR2 monoclonal antibodies were identified from an ELISA-based screening assay aimed at generating new TNFR2 antibodies. The antibodies were subsequently grouped on the basis of the epitope within TNFR2 bound by the antibody or fragment thereof. For the purposes of binning antibodies into categories characterized by similar epitope-binding specificity, the human TNFR2 amino acid sequence was considered in terms of its four cysteine-rich domains (CRDs): CRD1 (amino acid residues 48-76 of SEQ ID NO: 1), CRD2 (amino acid residues 78-120 of SEQ ID NO: 1), CRD3 (amino acid residues 121-162 of SEQ ID NO: 1), and CRD4 (amino acid residues 162-202 of SEQ ID NO: 1). The distribution of antibodies that bind one or more of these domains within TNFR2 is shown in Table 5, below.

TABLE 5
Epitope-binding properties of monoclonal TNFR2 antibodies
identified by ELISA-based screening assay

| Domain(s) within | |
|---|---|
| TNFR2 bound by antibody | Number of antibodies identified |
| CRD1 | 2 |
| CRD2 | 2 |
| CRD1 + CRD2 | 4 |
| CRD1 + CRD3 | 2 |
| CRD1 + CRD4 | 2 |
| CRD2 + CRD3 | 2 |
| CRD2 + CRD4 | 3 |
| CRD3 | 2 |
| CRD4 | 2 |
| CRD3 + CRD4 | 6 |

The antibodies identified from the screening assay were subsequently incubated with blood samples obtained from human patients suffering from advanced cutaneous T cell lymphoma in order to assess their effects on T-reg cells, effector T cells, and TNFR2+ cancer cells. After an incubation period, the quantities of effector T cells, T-reg cells, and TNFR2+ cancer cells were measured in each sample. The results of these experiments are reported in Table 6, below.

TABLE 6
Effects of monoclonal TNFR2 antibodies on T-reg cells,
effector T cells, and TNFR2+ cancer cells Epitope-
binding properties of monoclonal TNFR2 antibodies
identified by ELISA-based screening assay

| | | | Effector T | |
|---|---|---|---|---|
| Domain(s) within | Number of | T-reg cell | cell | Direct tumor |
| TNFR2 bound by | antibodies | killing | proliferation | cell killing |
| antibody | identified | observed? | observed? | observed? |
| CRD1 | 2 | No | No | No |
| CRD2 | 2 | No | No | No |
| CRD1 + CRD2 | 4 | No | No | No |
| CRD1 + CRD3 | 2 | No | No | No |
| CRD1 + CRD4 | 2 | No | No | No |
| CRD2 + CRD3 | 2 | Yes | No | No |
| CRD2 + CRD4 | 3 | No | No | No |
| CRD3 | 2 | Yes | No | No |
| CRD4 | 2 | Yes | No | No |
| CRD3 + CRD4 | 6 | Yes | Yes | Yes |

As shown in Table 6, antibodies that bound epitopes within portions of CRD3 and CRD4 were found to be capable of killing T-reg cells, inducing the proliferation of effector T cells, and directly killing tumor cells. Further, antibodies that specifically bound both CRD3 and CRD4 were found to be capable of exhibiting the foregoing characteristics even in the presence of TNFα, and are, thus, dominant TNFR2 antagonists. Taken together, these data demonstrate that antagonistic TNFR2 polypeptides (e.g., single-chain polypeptides, antibodies, antigen-binding fragments thereof, and constructs) that exhibit optimal antagonistic properties, such as the ability to simultaneously modulate a cancer patient's T-reg, T effector, and TNFR2+ cancer cell populations, even in the presence, for instance, of a TNFR2 agonist (e.g., TNFα or BCG), include those that specifically bind TNFR2 within CRD3 and CRD4, such as TNFRAB4.

Significantly, among the six antibodies that bound epitopes within portions of CRD3 and CRD4 was TNFRAB4, which does not bind to an epitope within residues 142-146 (KCRPG) of human TNFR2 (SEQ ID NO: 1) or an equivalent epitope within TNFR2 of a non-human mammal. Thus, surprisingly, antagonistic TNFR2 polypeptides (e.g., antibodies, antigen-binding fragments thereof, single-chain polypeptides, and constructs) can exhibit the ability to kill T-reg cells, induce effector T cell proliferation, and kill tumor cells without the need to bind an epitope within 142-146 (KCRPG) of human TNFR2 (SEQ ID NO: 1) or an equivalent epitope within TNFR2 of a non-human mammal.

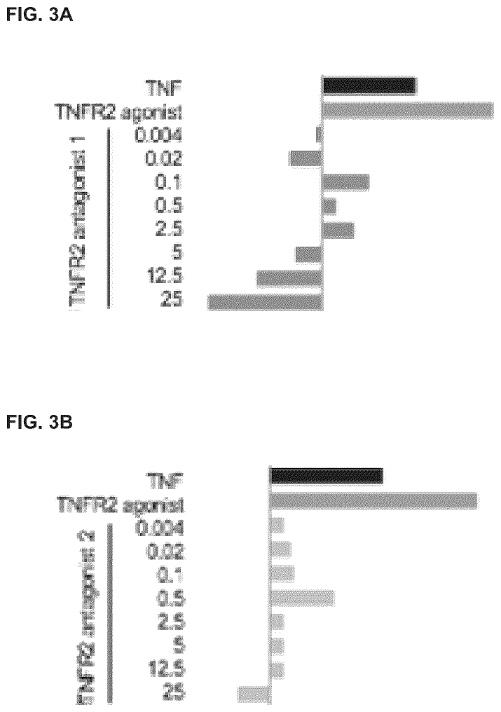

To further investigate these findings, the ability of a monoclonal TNFR2 antagonist antibody that specifically binds to TNFR2 within CRD3 and CRD4 to kill T-reg cells was compared to that of a monoclonal TNFR2 antagonist antibody that specifically binds TNFR2 within CRD2 and CRD3. Both antibodies were incubated with blood samples obtained from healthy human subjects, and after an incubation period, the quantities of effector T cells, T-reg cells, and TNFR2+ cancer cells were measured in each sample. The results of these experiments are shown in FIGS. 3A and 3B. These data demonstrate that TNFR2 antagonist antibodies, such as TNFRAB4, that bind TNFR2 within CRD3 and CRD4 (FIG. 3A) are capable of killing T-reg cells with a substantially greater potency than TNFR2 antagonist antibodies that bind TNFR2 within CRD2 and CRD3 only (FIG. 3B).

Collectively, the results of these investigations demonstrate the importance of epitopes within CRD4 of TNFR2 for promoting antagonistic activity, such as the killing of T-reg cells, expansion of T effector cells, and depletion of TNFR2+ cancer cells. These experiments have led to the discovery of epitope regions within CRD3 and CRD4 that direct antagonistic TNFR2 activity. For instance, particular epitopes within CRD3 and CRD4 that, when bound, impart the ability to exhibit optimal TNFR2 antagonistic activity include (i) those that contain one or more, or all, of residues 174-184 of TNFR2 (SSTDICRPHQI, SEQ ID NO: 31), (ii) those that contain one or more, or all, of residues 126-140 of TNFR2 (CALSKQEGCRLCAPL, SEQ ID NO: 32), and/or (iii) those that contain one or more, or all, of residues 156-165 of TNFR2 (TSDVVCKPC, SEQ ID NO: 33). The results of these studies demonstrate that antibodies that bind these epitopes, such as TNFRAB4, exhibit the ability to simultaneously kill T-reg cells, engender the proliferation of T effector cells, and kill cancer cells, and to exert these activities even in the presence of a TNFR2 agonist, rendering such antibodies dominant TNFR2 antagonists. Further, these activities can be effectuated by an antagonistic TNFR2 polypeptide without the need to bind an epitope within 142-146 (KCRPG) of human TNFR2 (SEQ ID NO: 1) or an equivalent epitope within TNFR2 of a non-human mammal.

### Materials and Methods for T-Reg Killing Assay

Human T-reg Flow^{T}M Kit (BioLegend, Cat. No. 320401)
   Cocktail Anti-human CD4 PE-Cy5/CD25 PE (BioLegend, Part No. 78930)
   Alexa Fluor® 488 Anti-human FOXP3, Clone 259D (BioLegend, Part No. 79467)
   Alexa Fluor® 488 Mouse IgG1, k Isotype Ctrl (ICFC), Clone MOPC-21 (BioLegend, Part No. 79486)
   FOXP3 Fix/Perm Buffer (4X) (BioLegend, Cat. No. 421401)
   FOXP3 Perm Buffer (10X) (BioLegend, Cat. No. 421402)
PE anti-human CD25, Clone: BC96 (BioLegend, Cat. No. 302606)
Alexa Fluor® 488 Anti-human FOXP3, Clone 259D (BioLegend, Cat. No. 320212)
PBS pH 7.4 (1X) (Gibco Cat. No. 10010-023)
HBSS (1X) (Gibco Cat. No. 14175-095)
FBS (heat inactivated)
15 ml tubes
Bench top centrifuge with swing bucket rotor for 15 ml tubes (set speed 1100 rpm or 200 g)

Monoclonal TNFR2 antibodies were tested for the ability to kill T-reg cells. Cultured T-reg cells were treated with varying concentrations of the TNFR2 antibodies in the presence and absence of stimulatory growth factors (e.g., TNFα) for set periods of time. As controls, T-reg cells were also incubated with TNFα alone at concentrations ranging from 0-40 ng/ml in order to select levels of TNFα that induce a high fractional increase in T-reg cell count. Additionally, T-reg cells were cultured in the presence of IL-2 alone.

Following the incubation of T-reg cells under the conditions described above, the cell counts were determined using flow cytometry analysis. T-reg cells at a density of 0.2-1×10⁶ cells/100 μl were distributed into a 15-ml conical tube and centrifuged for 5 minutes in order to pellet the cells. The supernatant was discarded and cells were resuspended in 100 μl of wash buffer (1x HBSS containing 2% FBS). 5 μl of PE anti-human CD25 fluorophore-antibody conjugate were added to this mixture, and the cells were subsequently vortexed and incubated in the dark for 25 minutes. The cells were then washed by adding 1 ml of wash buffer and subsequently centrifuging for 5 minutes. The supernatant was then discarded and 1 ml of FoxP3 fixation/permeabilization buffer (1:4 dilution of 4x FOXP3 Fix/Perm buffer in PBS) was added to the cells. The cells were then vortexed and incubated in the dark for 20 minutes. Cells were subsequently centrifuged for 5 minutes and supernatant was discarded. Cells were then resuspended in 1 ml of fresh wash buffer, vortexed, and centrifuged for 5 minutes. Cells were subsequently resuspended in 1 ml of 1x FOXP3 Perm Buffer (1:10 dilution of 10x FOXP3 Perm Buffer in PBS), vortexed, and incubated in the dark for 15 minutes. Following incubation, cells were centrifuged for 5 minutes and supernatant was subsequently discarded. The cell pellet was then resuspended in 100 μl of 1x FOXP3 Perm Buffer. At this point, 5 μl of either Alexa Fluor® 488 anti-human FOXP3 or Alexa Fluor® 488 mouse IgG1, k isotype control were added to the cells. Cells were then vortexed and incubated in the dark for 35 minutes. Following incubation, cells were washed by adding 1 ml of fresh wash buffer to the cells, vortexing the cells and centrifuging for 5 minutes. The supernatant was then discarded and the cell pellet was resuspended in 0.2-0.5 ml of 1x HBSS free of FBS. Cell counts were then determined by flow cytometry analysis.

### Example 3. Antagonistic TNFR2 Polypeptides that Bind Epitopes within Both CRD3 and CRD4 Exhibit the Ability to Kill Various TNFR2+ Cancer Cells

Antagonistic TFNR2 polypeptides that bind epitopes within CRD3 and CRD4 of human TNFR2, such as TNFRAB4, are capable of directly killing cancer cells that express TNFR2 antigen. To further investigate this activity, the effects of an antagonistic TNFR2 monoclonal antibody that binds epitopes within both CRD3 and CRD4 on human SW480 colon cancer cells, human cutaneous T cell lymphoma cells, and human ovarian cancer cells were assessed.

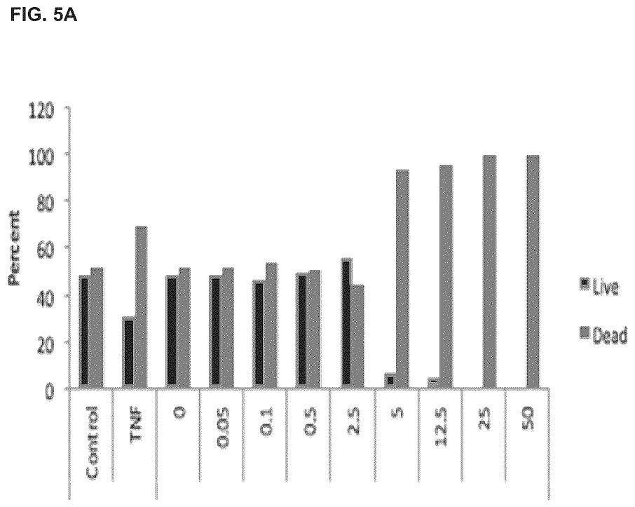

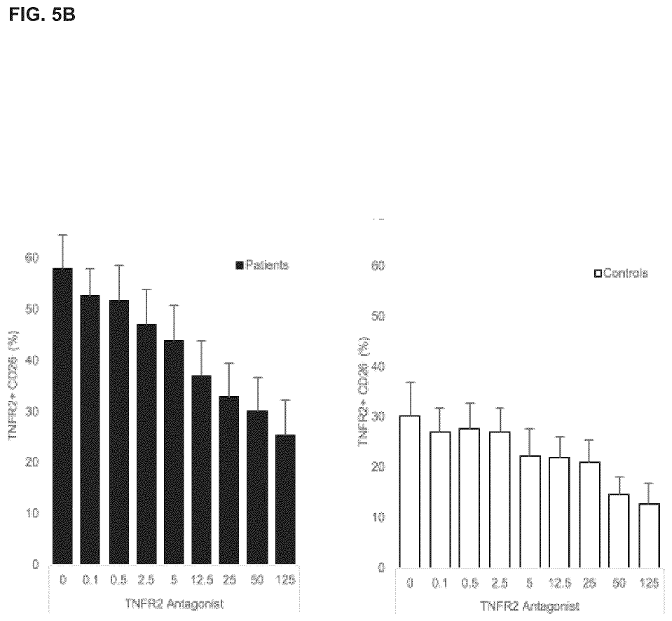

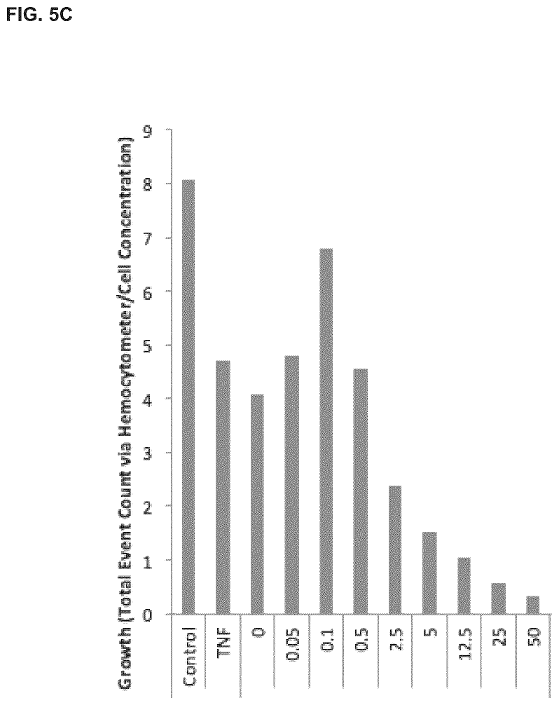

To examine the effects of an antagonistic TNFR2 monoclonal antibody that binds epitopes within both CRD3 and CRD4 on human SW480 colon cancer cells, samples of colon cancer cells were withdrawn from a human patient suffering from this disease, and the cells were subsequently incubated with various concentrations of the antagonist antibody (ranging from 0-50 pg/ml) for 7 days. As controls, populations of the isolated cells were incubated in parallel with a TNFR2 antibody that does not exhibit antagonistic activity, as well as with a TNFR2 agonist, TNFα. At the conclusion of the 7-day incubation period, the quantities of living and dead SW480 cancer cells were determined using flow cytometry techniques known in the art. The results of these experiments are shown in FIG. 5A. These data demonstrate that antagonistic TNFR2 antibodies that bind epitopes within both CRD3 and CRD4 of TNFR2 directly kill human colon cancer cells in a dose-dependent manner.

To examine the effects of an antagonistic TNFR2 monoclonal antibody that binds epitopes within both CRD3 and CRD4 on human cutaneous T cell lymphoma cells, samples of these cells were withdrawn from a human patient suffering from cutaneous T cell lymphoma, and the cells were subsequently incubated with various concentrations of the antagonist antibody (ranging from 0-125 pg/ml) for from 48-72 hours. As controls, samples of CD26-TNFR2+ cells were withdrawn from healthy human subjects, and these cells were subsequently incubated with the same antagonist antibody for the same incubation period and at the same concentrations as those used for the cutaneous T cell lymphoma cells. At the conclusion of the incubation period, the quantities of TNFR2+CD26− cells in each sample were determined. This measurement provides a readout of cancer cell death in patients suffering from T cell lymphoma, as T cell lymphoma cells exhibit a TNFR2+CD26− immunophenotype. Additionally, this measurement enables one to determine the effect of the TNFR2 antagonist antibody on TNFR2-expressing cells in a healthy human subject. The results of these experiments are shown in FIG. 5B. These data demonstrate that antagonistic TNFR2 antibodies that bind epitopes within both CRD3 and CRD4 of TNFR2 directly kill human T cell lymphoma cells in a dose-dependent manner, even at low doses. Additionally, these results demonstrate that the effects of antagonistic TNFR2 antibodies that bind epitopes within CRD3 and CRD4 on TNFR2+ cells are substantially more potent in patients suffering from cancer relative to healthy human subjects.

Finally, to examine the effects of an antagonistic TNFR2 monoclonal antibody that binds epitopes within both CRD3 and CRD4 on human ovarian cancer cells, samples of these cells were withdrawn from a human patient suffering from ovarian cancer, and the cells were subsequently incubated with various concentrations of the antagonist antibody (ranging from 0-50 pg/ml) for from 5-7 days. As controls, populations of the isolated cells were incubated in parallel with a TNFR2 antibody that does not exhibit antagonistic activity, as well as with a TNFR2 agonist, TNFα. At the conclusion of the incubation period, the quantities of living and dead ovarian cancer cells were determined using hemocytometry techniques known in the art. The results of these experiments are shown in FIG. 5C. These data demonstrate that antagonistic TNFR2 antibodies that bind epitopes within both CRD3 and CRD4 of TNFR2 directly kill human ovarian cancer cells in a dose-dependent manner.

### Example 4. Generating Antagonistic TNFR2 Antibodies by Phage Display

An exemplary method for in vitro protein evolution of antagonistic TNFR2 antibodies described herein is phage display, a technique which is well known in the art. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations can be introduced may be, e.g., a naïve human germline sequence as described herein. These mutations can be performed using standard mutagenesis techniques described herein or known in the art. Each mutant sequence thus encodes an antibody corresponding in overall structure to the template except having one or more amino acid variations in the sequence of the template. Retroviral and phage display vectors can be engineered using standard vector construction techniques as described herein or known in the art. P3 phage display vectors along with compatible protein expression vectors, as is well known in the art, can be used to generate phage display vectors for antibody diversification as described herein.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its structure.

In a typical screen, a phage library is contacted with and allowed to bind a TNFR2-derived peptide (e.g., a peptide having the sequence of any one of SEQ ID NOs: 31-33), or a particular subcomponent thereof. To facilitate separation of binders and non-binders, it is convenient to immobilize the target on a solid support. Phage bearing a TNFR2-binding moiety can form a complex with the target on the solid support whereas non-binding phage remain in solution and can be washed away with excess buffer. Bound phage can then liberated from the target by changing the buffer to an extreme pH (pH 2 or pH 10), changing the ionic strength of the buffer, adding denaturants, or other known means. To isolate the binding phage, a protein elution can be performed.

The recovered phage can then be amplified through infection of bacterial cells and the screening process can be repeated with the new pool that is now depleted in non-binding antibodies and enriched for antibodies that bind the target peptide. The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof, typically 10-50 out of about 10⁹ to 10¹⁰ original candidates from each library. An increase in the number of phage recovered at each round of selection is a good indication that convergence of the library has occurred in a screen. After a set of binding polypeptides is identified, the sequence information can be used to design other secondary phage libraries, biased for members having additional desired properties (See WO 2014/152660; the disclosure of which is incorporated herein by reference).

### Example 5. Producing an scFv TNFR2 Antagonist

Antibody fragments described herein include scFv fragments, which contain the antibody variable regions of the light and heavy chains combined in a single peptide chain. A monoclonal antagonistic TNFR2 antibody described herein, such as TNFRAB4, can be used as a framework for the development of a scFv antibody fragment by recombinantly expressing a polynucleotide encoding the variable region of a light chain of the TNFR2 antibody operatively linked to the variable region of a heavy chain of that antibody. The polynucleotide encoding the variable region of the heavy chain may contain one or more, or all, of the CDRs of TNFRAB4, or one or more, or all, of the of the CDR-H1, CDR-H2, CDR-H3, CDR-L1, CDR-L2, and CDR-L3 sequences in which one or more, or all, of the CDR sequences exhibits at least 85% sequence identity (e.g., 90%, 95%, 96%, 97%, 98%, 99%, or 100% sequence identity) to the corresponding CDR sequence of TNFRAB4) or contains one or more (for instance, up to 3) amino acid substitutions (e.g., one or more conservative amino acid substitutions) relative to the corresponding CDR sequence of TNFRAB4. Recombinant polynucleotides encoding such variable regions can be produced, for example, using established mutagenesis protocols as described herein or known in the art. This polynucleotide can then be expressed in a cell (e.g., a CHO cell) and the scFv fragment can subsequently be isolated from the cell culture media.

Alternatively, scFv fragments derived from a TNFR2 antagonist can be produced by chemical synthetic methods (e.g., by Fmoc-based solid-phase peptide synthesis, as described herein). One of skill in the art can chemically synthesize a peptide chain consisting of the variable region of a light chain of a monoclonal antagonistic TNFR2 antibody described herein, such as TNFRAB4, operatively linked to the variable region of a heavy chain of the antibody. Native chemical ligation can be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference.

### Example 6. Producing a Humanized Antagonistic TNFR2 Antibody

One method for producing humanized TNFR2 antibodies described herein is to import one or more, or all, of the CDRs of a non-human antagonistic TNFR2 antibody, such as TNFRAB4, into a human antibody consensus sequence. Consensus human antibody heavy chain and light chain sequences are known in the art (see e.g., the “VBASE” human germline sequence database; Kabat et al. (Sequences of Proteins of Immunological Interest, Fifth Edition, U.S. Department of Health and Human Services, NIH Publication No. 91-3242, 1991); Tomlinson et al. (J. Mol. Biol. 227:776-798, 1992); and Cox et al. (Eur. J. Immunol. 24:827-836, 1994); the disclosures of each of which are incorporated herein by reference). Using established procedures, one can identify the variable domain framework residues and CDRs of a consensus antibody sequence (e.g., by sequence alignment (see Kabat, supra)). One can substitute, e.g., one or more, or all, of the CDR-H1, CDR-H2, CDR-H3, CDR-L1, CDR-L2, and CDR-L3 sequences of the consensus antibody with the corresponding CDR sequence(s) of a non-human antagonistic TNFR2 antibody described herein, such as TNFRAB4, in order to produce a humanized, antagonistic TNFR2 antibody described herein. Polynucleotides encoding the above-described CDRs sequences can be produced synthetically or recombinantly, e.g., using gene editing techniques described herein or known in the art.

One example of a variable domain of a consensus human antibody includes the heavy chain variable domain EVQLVESGGGLVQPGGSLRLSCAASGFTFSDYAMSWVRQAPGKGLEWVAVISENGSDTYYADSVKGRFTIS RDDSKNTLYLQMNSLRAEDTAVYYCARDRGGAVSYFDVWGQGTLVTVSS (SEQ ID NO: 10) and the light chain variable domain DIQMTQSPSSLSASVGDRVTITCRASQDVSSYLAWYQQKPGKAPKLLIYAASSLESGVPSRFSGSGSGTDFT LTISSLQPEDFATYYCQQYNSLPYTFGQGTKVEIKRT (SEQ ID NO: 11), identified in U.S. Pat. No. 6,054,297; the disclosure of which is incorporated herein by reference (CDRs are shown in bold). In order to produce humanized, antagonistic TNFR2 antibodies, one can recombinantly express a polynucleotide encoding the above consensus sequence in which one or more, or all, of the CDR-H1, CDR-H2, CDR-H3, CDR-L1, CDR-L2, and CDR-L3 sequences are replaced with the corresponding CDR sequences of a non-human antagonistic TNFR2 antibody described herein, such as TNFRAB4.

A polynucleotide encoding the above heavy chain and light chain variable domains operatively linked to one another can be incorporated into an expression vector (e.g., an expression vector optimized for protein expression in prokaryotic or eukaryotic cells as described herein or known in the art). The single-chain antibody fragment (scFv) can thus be expressed in a host cell and subsequently purified from the host cell medium or the host cell using established techniques, such as size-exclusion chromatography and/or affinity chromatography as described herein.

### Example 7. Treatment of Cancer in a Human Patient by Administration of Antagonistic TNFR2 Polypeptides

The antagonistic TNFR2 polypeptides (e.g., single-chain polypeptides, antibodies, antigen-binding fragments thereof, or constructs) described herein can be administered to a human patient in order to treat a cell proliferation disorder, such as cancer. Administration of these polypeptides suppresses the growth and proliferation of T-reg cells. Antibodies described herein can also be administered to a patient in order to suppress a T-reg-mediated immune response. For instance, a human patient suffering from cancer, e.g., a cancer described herein, can be treated by administering an antagonistic TNFR2 polypeptide described herein by an appropriate route (e.g., intravenously) at a particular dosage (e.g., between 0.001 and 100 mg/kg/day) over a course of days, weeks, months, or years. If desired, the antagonistic-TNFR2 polypeptide can be modified, e.g., by hyperglycosylation or by conjugation with PEG, so as to evade immune recognition and/or to improve the pharmacokinetic profile of the polypeptide.

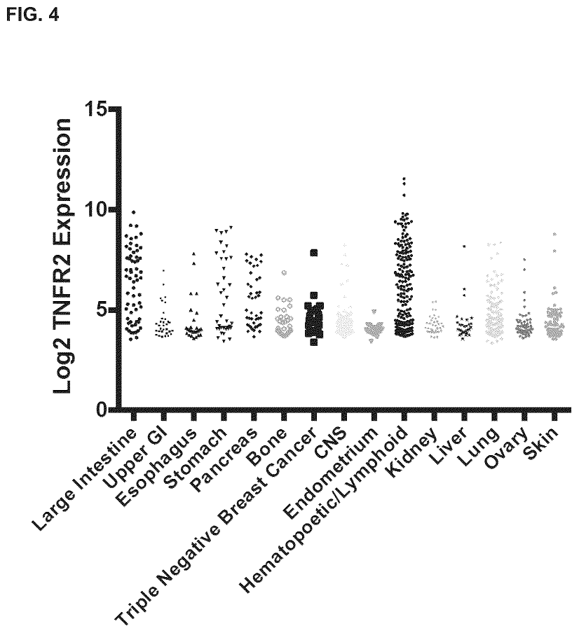

The cancer may be, for instance, one that is characterized by cells that express TNFR2, such as, for instance, Hodgkin's lymphoma, cutaneous non-Hodgkin's lymphoma, T cell lymphoma, ovarian cancer, colon cancer, multiple myeloma, renal cell carcinoma, skin cancer, lung cancer, liver cancer, endometrial cancer, a hematopoietic or lymphoid cancer, a central nervous system cancer (e.g., glioma, neuroblastoma, and other cancers of central nervous system cells described herein), breast cancer, pancreatic cancer, stomach cancer, esophageal cancer, and upper gastrointestinal cancer. In such instances, the antagonistic TNFR2 polypeptide may treat the cancer by one or more mechanisms. For example, the antagonistic TNFR2 polypeptide may bind TNFR2 on the surface of a T-reg cell, such as an activated T-reg cell expressing CD25Hi and CD45RA^{Low}, or a MDSC, thereby inhibiting the proliferation of, and/or directly killing, the T-reg cell or MDSC. The T-reg cells and/or MDSCs that are killed or for which proliferation is suppressed may be those that are located in the microenvironment of a tumor. The reduced population of T-reg cells and/or MDSCs effectuated by the antagonistic TNFR2 polypeptide may, in turn, enable the expansion of populations of tumor-reactive CD8+ cytotoxic T cells, which can mount an immune response against the cancerous cells. The antagonistic TNFR2 polypeptide may, Additionally, or alternatively, induce the direct expansion of CD8+ effector T cells. Additionally, or alternatively, the antagonistic TNFR2 polypeptide may bind TNFR2 on the surface of a TNFR2+ cancer cell, thereby inhibiting the proliferation of, and/or directly killing, the cancer cell. Exemplary cancers characterized by TNFR2+ cancer cells are shown in FIG. 4.

The progression of the cancer that is treated with an antagonistic TNFR2 polypeptide described herein can be monitored by any one or more of several established methods. A physician can monitor the patient by direct observation in order to evaluate how the symptoms exhibited by the patient have changed in response to treatment. A patient may also be subjected to MRI, CT scan, or PET analysis in order to determine if a tumor has metastasized or if the size of a tumor has changed, e.g., decreased in response to treatment with an anti-TNFR2 antibody described herein. Optionally, cells can be extracted from the patient and a quantitative biochemical analysis can be conducted in order to determine the relative cell-surface concentrations of various growth factor receptors, such as the epidermal growth factor receptor. Based on the results of these analyses, a physician may prescribe higher/lower dosages or more/less frequent dosing of the antagonistic TNFR2 polypeptide in subsequent rounds of treatment.

### Example 8. Treatment of HIV in a Human Patient by Administration of Antagonistic TNFR2 Polypeptides

The antagonistic TNFR2 polypeptides (e.g., single-chain polypeptides, antibodies, antigen-binding fragments thereof, and constructs) described herein can be administered to a human patient in order to treat a viral infection, such as HIV. Administration of these polypeptides can, for instance, suppress the growth and proliferation of T-reg cells and MDSCs, which can enhance the immune response of a patient by allowing the expansion of cytotoxic T lymphocytes capable of mounting an attack on infected cells. For instance, a human patient suffering from HIV can be treated by administering an antagonistic TNFR2 polypeptide described herein by an appropriate route (e.g., intravenously) at a particular dosage (e.g., between 0.001 and 100 mg/kg/day) over a course of days, weeks, months, or years. If desired, the polypeptide can be modified, e.g., by hyperglycosylation or by conjugation with PEG, so as to evade immune recognition and/or to improve the pharmacokinetic profile of the polypeptide.

The progression of HIV that is treated with an antagonistic TNFR2 polypeptide described herein can be monitored by any one or more of several established methods. A physician can monitor the patient by direct observation in order to evaluate how the symptoms exhibited by the patient have changed in response to treatment. A blood sample can also be withdrawn from the patient in order to analyze the cell count of one or more white blood cells in order to determine if the quantity of infected cells has changed (e.g., decreased) in response to treatment with an antagonistic TNFR2 polypeptide described herein. Based on the results of these analyses, a physician may prescribe higher/lower dosages or more/less frequent dosing of the antagonistic TNFR2 polypeptide in subsequent rounds of treatment.

### Example 9. Treatment of Mycobacterium tuberculosis in a Non-Human Mammal by Administration of Antagonistic TNFR2 Polypeptides

The antagonistic TNFR2 polypeptides (e.g., single-chain polypeptides, antibodies, antigen-binding fragments thereof, and constructs) described herein can be administered to a non-human mammal (e.g., a bovine mammal, pig, bison, horse, sheep, goat, cow, cat, dog, rabbit, hamster, guinea pig, or other non-human mammal) in order to treat a bacterial infection, such as Mycobacterium tuberculosis. Administration of these polypeptides may, for instance, suppress the proliferation of, and/or directly kill, T-reg cells and/or MDSCs, which can enhance the immune response of a patient by allowing the expansion of cytotoxic T lymphocytes capable of mounting an attack on the pathogenic organism. For instance, a non-human mammal suffering from Mycobacterium tuberculosis can be treated by administering an antagonistic TNFR2 polypeptide described herein by an appropriate route (e.g., intravenously) at a particular dosage (e.g., between 0.001 and 100 mg/kg/day) over a course of days, weeks, months, or years. If desired, the antagonistic TNFR2 polypeptide can be modified, e.g., by hyperglycosylation or by conjugation with PEG, so as to evade immune recognition and/or to improve the pharmacokinetic profile of the polypeptide.

The progression of the Mycobacterium tuberculosis infection that is treated with an antagonistic TNFR2 polypeptide described herein can be monitored by any one or more of several established methods. A physician can monitor the patient by direct observation in order to evaluate how the symptoms exhibited by the patient have changed in response to treatment. A blood sample can also be withdrawn from the patient in order to analyze the cell count of one or more white blood cells in order to determine if the immune response has changed (e.g., increased) in response to treatment with an antagonistic TNFR2 polypeptide described herein. Based on the results of these analyses, a physician may prescribe higher/lower dosages or more/less frequent dosing of the antagonistic TNFR2 polypeptide in subsequent rounds of treatment.

### Example 10. Treatment of Cancer or an Infectious Disease in a Human Patient by Administration of Antagonistic TNFR2 Polypeptides in Combination with an Immunotherapy Agent

The antagonistic TNFR2 antibodies, antigen-binding fragments, single-chain polypeptides, and constructs described herein can be administered to a human patient in combination with (for instance, admixed with, co-administered with, or administered separately from) an immunotherapy agent in order to treat a cell proliferation disorder, such as cancer, or an infectious disease, such as a viral, bacterial, fungal, or parasitic infection. Administration of the antibody, antigen-binding fragment, single-chain polypeptide, or construct can suppress the growth and proliferation of T-reg cells and/or cancer cells that express TNFR2. Immunotherapy agents, such as anti-CTLA-4 agents (e.g., an anti-CTLA-4 antibody or antigen-binding fragment thereof, such as ipilimumab and tremelimumab), anti-PD-1 agents (e.g., an anti-PD-1 antibody or antigen-binding fragment thereof, such as nivolumab, pembrolizumab, avelumab, durvalumab, and atezolizumab), anti-PD-L1 agents (e.g., atezolizumab and avelumab), anti-PD-L2 agents, TNF-α cross-linking agents, TRAIL cross-linking agents, anti-CD27 agents, anti-CD30 agents, anti-CD40 agents, anti-4-1 BB agent, anti-GITR agents, anti-OX40 agents, anti-TRAILR1 agents, anti-TRAILR2 agent, and anti-TWEAKR agents can function in tandem with antagonist TNFR2 antibodies, antigen-binding fragments thereof, single-chain polypeptides, or constructs, as immunotherapy agents are capable of downregulating the signal transduction of immune checkpoint proteins (e.g., immune checkpoint receptors and/or ligands) that would otherwise lead to tolerance toward tumor-associated antigens and downregulation of the cytotoxic T cell response. Additional examples of immunotherapy agents that may be used in conjunction with an antagonistic TNFR2 antibody, antigen-binding fragment thereof, single-chain polypeptide, or construct include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat.

A physician of skill in the art may administer an antibody, antigen-binding fragment thereof, single-chain polypeptide, or construct that specifically binds TNFR2 as an antagonist, for instance, as described herein, to a human patient suffering from a cancer or infectious disease in combination with an immunotherapy agent. The antibody, antigen-binding fragment thereof, single-chain polypeptide, or construct and the immunotherapy agent may be administered to the patient by an appropriate route of administration (for example, intravenously, intramuscularly, or subcutaneously, among others) at a particular dosage (for example, between 0.001 and 100 mg/kg/day, among other ranges) over a course of days, weeks, months, or years. If desired, the anti-TNFR2 antibody, antigen-binding fragment, single-chain polypeptide, or construct can be modified, for instance, by hyperglycosylation or by conjugation with PEG, so as to evade immune recognition and/or to improve the pharmacokinetic profile of the antibody, antigen-binding fragment, single-chain polypeptide, or construct.

The progression of the cancer or infectious disease that is treated in this fashion can be monitored by any one or more of several established methods. A physician can monitor the patient by direct observation in order to evaluate how the symptoms exhibited by the patient have changed in response to treatment. A patient may also be subjected to MRI, CT scan, or PET analysis in order to determine if a tumor has metastasized or if the size of a tumor has changed, for example, decreased in response to treatment with an anti-TNFR2 antibody, antigen-binding fragment, single-chain polypeptide, or construct and an immunotherapy agent. Optionally, cells can be extracted from the patient and a quantitative biochemical analysis can be conducted in order to determine the relative cell-surface concentrations of various growth factor receptors, such as the epidermal growth factor receptor. Based on the results of these analyses, a physician may prescribe higher/lower dosages or more/less frequent dosing of the antagonistic TNFR2 antibody, antigen-binding fragment, single-chain polypeptide, or construct and immunotherapy agent in subsequent rounds of treatment.

### Example 11. Treatment of Cancer in a Murine Model by Administration of an Antagonistic TNFR2 Antibody Alone or in Combination with an Anti-PD-1 Antibody or an Anti-CTLA-4 Antibody

An antagonistic TNFR2 antibody alone or in combination with an anti-PD-1 antibody or an anti-CTLA-4 antibody can be used to reduce cancer progression (e.g., colon cancer progression) and increase survival in a subject with cancer. A series of experiments was conducted using murine models of colon cancer to investigate the effects on cancer progression of combination therapy using an immunotherapy agent (e.g., an anti-PD1 antibody or an anti-CTLA4 antibody) and an antagonistic TNFR2 antibody (e.g., an antagonist TNFR2 antibody that binds murine TNFR2 at an epitope defined by one or more amino acids within cysteine rich domain (CRD) 3 (CRD3) and an epitope defined by one or more amino acids within CRD4),. The antagonist TNFR2 antibody used in these experiments specifically binds murine TNFR2 at epitopes within amino acid residues 125-141, 152-168, and 172-189. The amino acid sequence of murine TNFR2 is depicted in alignment with the amino acid sequence of human TNFR2 in FIG. 6. As evidenced by FIG. 6, the epitopes bound by the murine antibody used in these experiments align well with amino acid residues 126-140 (CALSKQEGCRLCAPL) of SEQ ID NO: 1, residues 156-165 (TSDVVCKPCA) of SEQ ID NO: 1, and 174-184 (SSTDICRPHQI) of SEQ ID NO: 1 in human TNFR2.

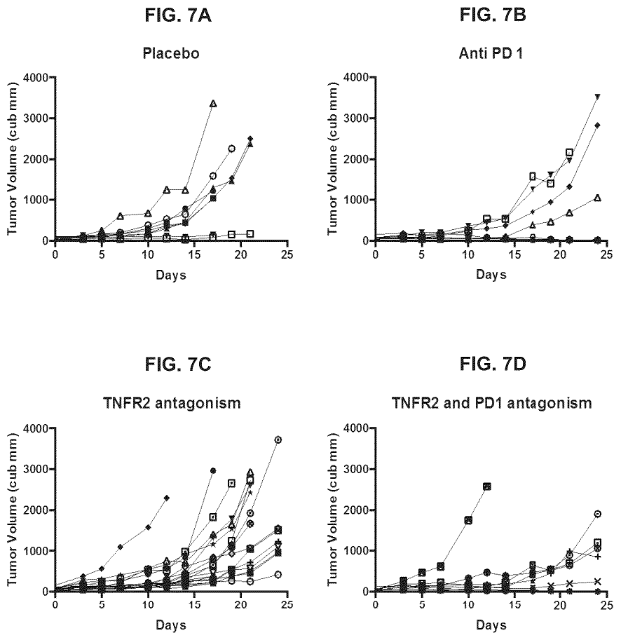

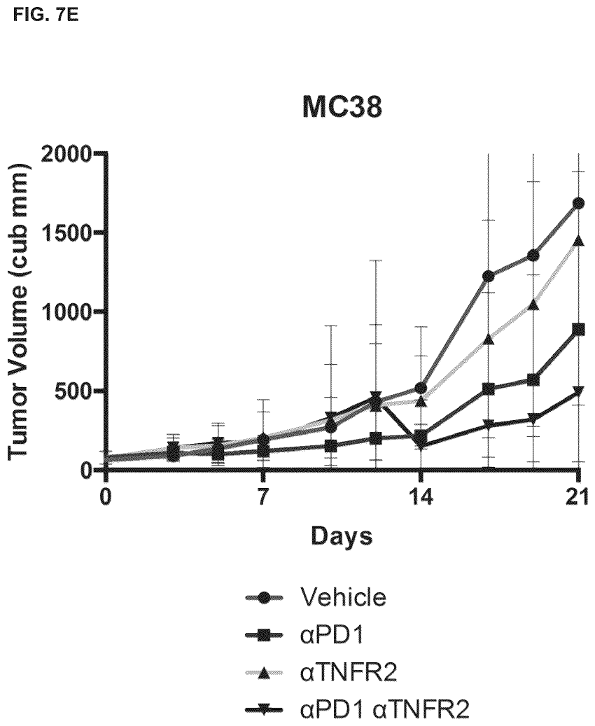

In a first experiment, MC38 mice, a model organism exhibiting colon cancer, were obtained and separated into four treatment arms. These groups were administered either placebo, an antagonistic TNFR2 antibody, an anti-PD-1 antibody, or both an antagonistic TNFR2 antibody and an anti-PD-1 antibody. The anti-PD1 antibody used in these studies, Clone RMP1-14, was purchased from BioXCell (West Lebanon, NH). Tumor volume in each mouse was subsequently monitored over time. The effects of these four treatment regimens on tumor volume in MC38 mice are shown in FIGS. 7A-7E. These data demonstrate that, among all treatment arms tested, the combination of an antagonistic TNFR2 antibody and an anti-PD-1 antibody resulted in the greatest suppression of tumor growth. Moreover, as shown in FIG. 7E, it was observed that combined treatment of MC38 mice with an antagonistic TNFR2 antibody and an anti-PD-1 antibody resulted in a synergistic decrease in tumor growth.

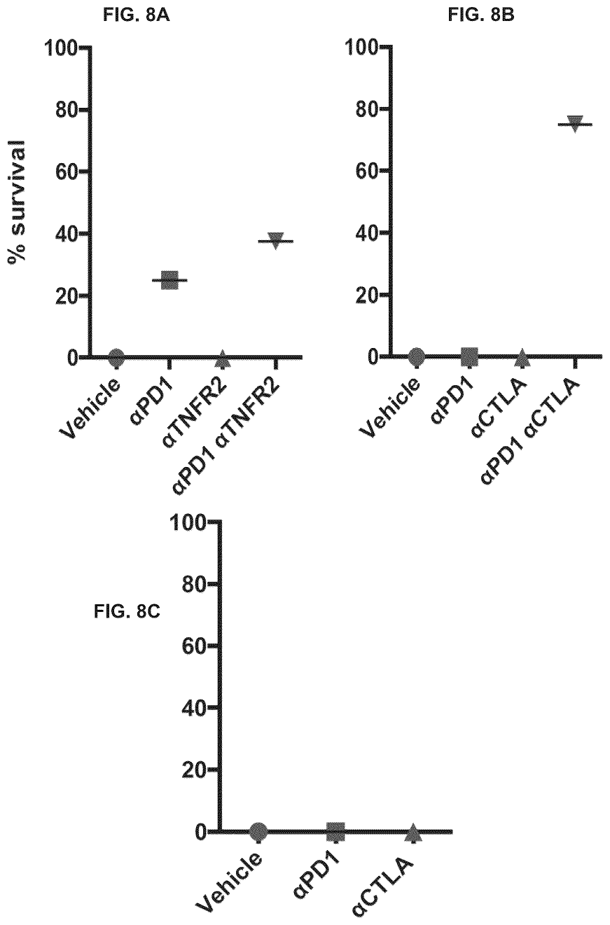

The synergistic effect observed upon treatment of MC38 mice with an anti-PD-1 antibody and an antagonistic TNFR2 antibody is also manifest in improved survival rates. As shown in FIG. 8A, among the four treatment arms described above, MC38 mice exhibited the highest rate of survival when administered an antagonistic TNFR2 antibody in combination with an anti-PD-1 antibody. For comparison, data showing the survival rates of MC38 mice upon administration of anti-PD-1 and anti-CTLA-4 antibodies, either alone or in combination, are shown in FIGS. 8B and 8C. Mice receiving combined therapy with an antagonistic TNFR2 antibody and a PD-1 antibody did not exhibit any signs of toxicity or autoimmunity. This result is significant, as MC38 mice previously treated with a combination of an anti-PD-1 antibody and an anti-CTLA-4 antibody were found to exhibit high toxicity and adverse autoimmune reactivity. Taken together, these results demonstrate that combined treatment with an antagonistic TNFR2 antibody and an anti-PD-1 antibody promotes a robust and low-toxicity anti-cancer effect. This effect is greater than that observed upon treatment with an immunotherapy agent alone.

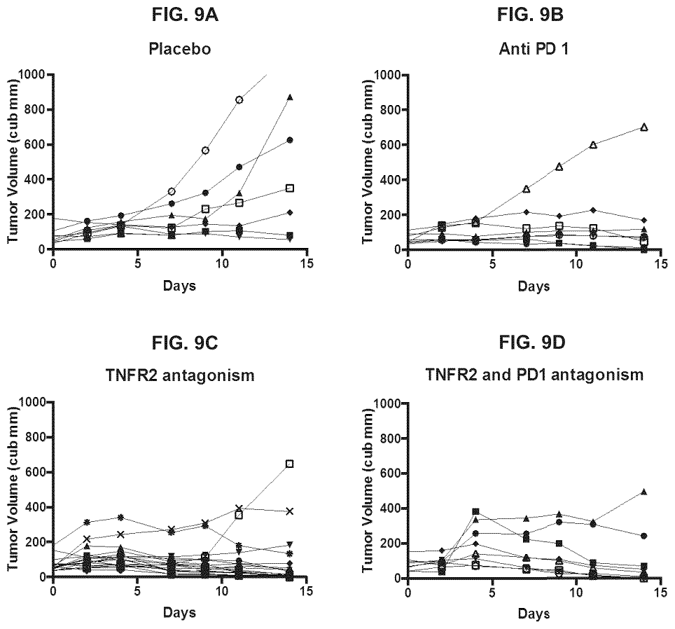

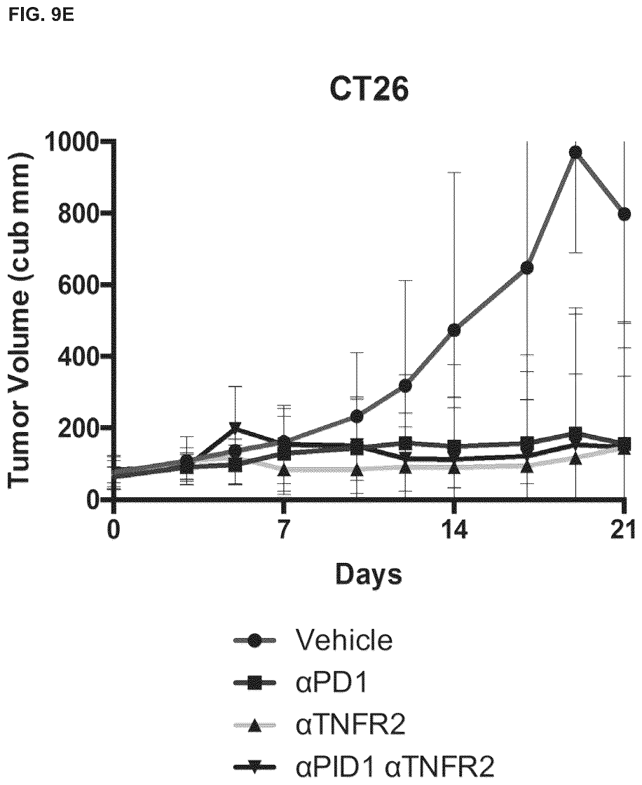

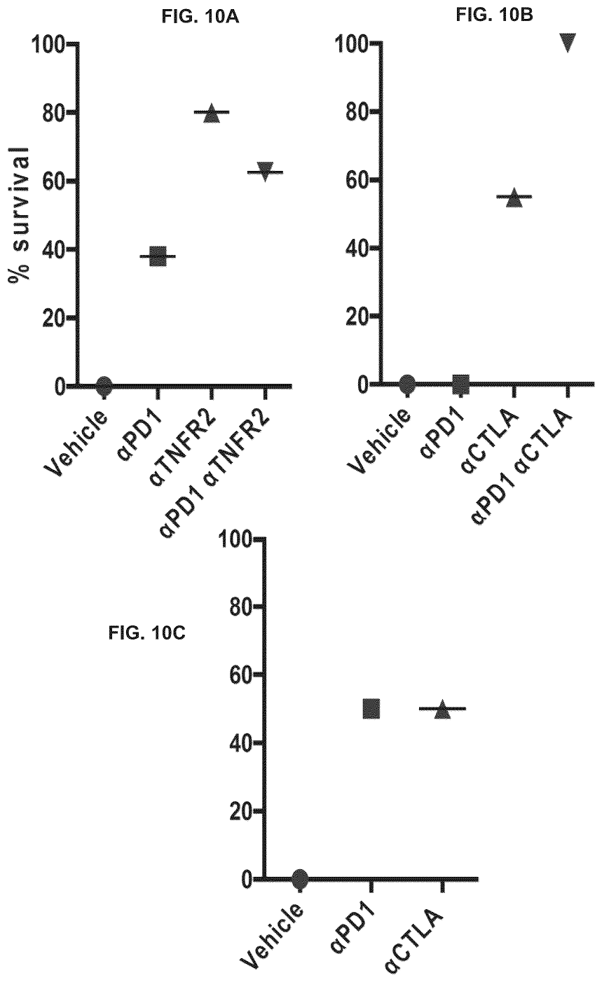

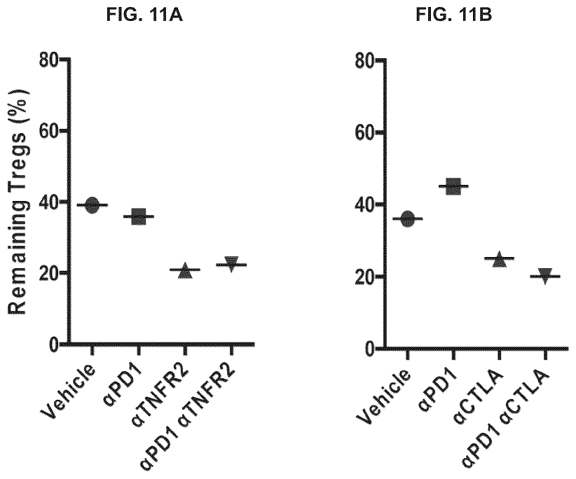

To further explore the effects of an antagonistic TNFR2 antibody alone and in combination with an immunotherapy agent, the experiments described above were repeated with CT26 mice, which is a second model of colon cancer. As shown in FIGS. 9A-9E, treatment of these mice with an antagonistic TNFR2 antibody resulted in a significant reduction of tumor growth relative to placebo treatment. Additionally, as shown in FIGS. 10A-10C, treatment with an antagonistic TNFR2 antibody, both alone and in combination with anti-PD-1 antibody treatment, resulted in improved survival rates in CT26 mice relative to control treatment (vehicle alone). These data also show that administration of an antagonistic TNFR2 antibody as a single treatment resulted in a greater improvement in murine survival relative to anti-PD-1 or anti-CTLA-4 antibody treatment alone. Treatment of CT26 mice with an antagonistic TNFR2 antibody, both alone and in combination with an anti-PD-1 antibody, also resulted in diminished Treg survival. In fact, almost all tumor infiltrating Tregs were eliminated with the single arm anti-TNFR2 antibody treatment (FIGS. 11A and 11B). These figures also show that antagonistic TNFR2 antibody treatment is as capable as anti-CTLA-4 antibody treatment in eliminating infiltrating Treg cells. Additionally, treatment with an anti-PD-1 antibody alone seems to have no impact on infiltrating Treg cells. These results further demonstrate the beneficial synergistic effect engendered upon combining antagonistic TNFR2 antibody treatment with an immunotherapy agent, such as an anti-PD-1 antibody. These results also show that antagonistic TNFR2 antibodies and antigen-binding fragments thereof may function in humans as a stand-alone therapy. To determine which of the tumors studied here might most benefit from a combined therapy versus a stand-alone TNFR2 antagonist therapy, it was documented that CT26 tumors features oncogene expression of TNFR2 as well as high quantities of Tregs expressing TNFR2. Due to these properties, CT26 tumors may be particularly well suited for stand-alone antagonist TNFR2 therapy. Using these biomarkers of the tumor microenvironment, one may determine whether a particular tumor is particularly suited for stand-alone therapy or combination therapy. In this way, antagonistic TNFR2 antibody therapy can be customized for greater efficacy.

Collectively, these results demonstrate that antagonistic TNFR2 antibodies, either alone or in combination with an immunotherapy agent(s) (e.g., an anti-PD-1 antibody), are capable of reducing tumor volume, suppressing Treg infiltration, and improving survival in murine colon cancer models, which are indicative of a treatment effect in humans. Moreover, antagonistic TNFR2 antibody treatment was shown to improve the potency of two different immunotherapy agents in two distinct models of cancer.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

1. An antibody or antigen-binding fragment thereof that specifically binds human tumor necrosis factor receptor 2 (TNFR2) at an epitope defined by one or more amino acids within cysteine rich domain (CRD) 3 (CRD3) and/or an epitope defined by one or more amino acids within CRD4, wherein the antibody or antigen-binding fragment thereof does not bind an epitope of TNFR2 defined by one or more of amino acids 142-146 (KCRPG) of SEQ ID NO: 1.

2. A method of identifying a TNFR2 antagonist antibody or antigen-binding fragment thereof comprising:
(a) exposing a heterogeneous mixture of antibodies or fragments thereof to at least one peptide having the amino acid sequence of any one of SEQ ID NOs: 31-33; and
(b) retaining antibodies or fragments thereof that specifically bind the peptide and removing antibodies or fragments thereof that do not specifically bind the peptide, thereby producing an enriched antibody mixture comprising at least one the TNFR2 antagonist antibody or antigen-binding fragment thereof.

3. A method of producing a TNFR2 antagonist antibody or antigen-binding fragment thereof comprising immunizing a non-human mammal with a peptide comprising the sequence of any one of SEQ ID NOs: 31-33 and collecting serum comprising the TNFR2 antagonist antibody or antigen-binding fragment thereof.

4. An antibody or antigen-binding fragment thereof that is produced by the method of claim 3.

5. A single-chain polypeptide that competitively inhibits the binding of TNFR2 to the antibody or antigen-binding fragment thereof of claim 1.

6. A construct comprising a first polypeptide domain and a second polypeptide domain, wherein the first polypeptide domain and the second polypeptide domain each independently comprise a single-chain polypeptide of claim 5.

7. A polynucleotide encoding the antibody or antigen-binding fragment thereof of claim 1.

8. A vector comprising the polynucleotide of claim 7.

9. An isolated host cell comprising the vector of claim 8.

10. A pharmaceutical composition comprising the antibody or antigen-binding fragment thereof of claim 1.

11. A method of producing the antibody or antigen-binding fragment thereof of claim 1 comprising expressing a polynucleotide encoding the antibody or antigen-binding fragment thereof in a host cell and recovering the antibody or antigen-binding fragment thereof from host cell medium or the host cell.

12. A method of producing the single-chain polypeptide of claim 5 comprising expressing a polynucleotide encoding the single-chain polypeptide in a host cell and recovering the single-chain polypeptide from host cell medium.

13. A method of producing the construct of claim 6 comprising expressing a polynucleotide encoding the construct in a host cell and recovering the construct from host cell medium or the host cell.

14. A method of inhibiting an immune response mediated by a regulatory T cell in a human comprising administering to the human the antibody or antigen-binding fragment thereof of claim 1.

15. A method of treating a cell proliferation disorder in a human comprising administering to the human the antibody or antigen-binding fragment thereof of claim 1.

16. A method of treating an infectious disease in a human comprising administering to the human the antibody or antigen-binding fragment thereof of claim 1.

17. A kit comprising the antibody or antigen-binding fragment thereof of claim 1.
